(19) United States
(12) Patent Application Publication
(10) Pub. No.: US 2003/0073701 A1
(43) Pub. Date: Apr. 17, 2003

(54) Succinoylamino heterocycles as inhibitors of a beta protein production

(76) Inventors: Lorin A. Thompson, Wilmington, DE (US); Padmaja Kasireddy, Kennett Square, PA (US)

Correspondence Address: BRISTOL-MYERS SQUIBB PHARMA COMPANY, PATENT DEPARTMENT, P.O. BOX 4000, PRINCETON, NJ, 08543-4000, US

(21) Appl. No.: 09/823,820

(22) Filed: Mar. 31, 2001

(51) Int. Cl. A61K 31/496; A61K 31/506; A61K 31/4545

(52) U.S. Cl. 514/255.01; 514/253.01; 514/252.14; 514/256; 514/330; 514/318; 514/343; 514/423; 544/295; 544/360; 544/386; 544/333; 546/208

(57) ABSTRACT

This invention relates to novel succinoylamino heterocycles having drug and bio-affecting properties, their pharmaceutical compositions and methods of use. These novel compounds inhibit the processing of amyloid precursor protein and, more specifically, inhibit the production of Aβ-peptide, thereby acting to prevent the formation of neurological deposits of amyloid protein. More particularly, the present invention relates to the treatment of neurological disorders related to β-amyloid production such as Alzheimer's disease and Down's Syndrome.

### FIELD OF THE INVENTION

This invention relates to novel succinoylamino heterocycles having drug and bio-affecting properties, their pharmaceutical compositions and methods of use. These novel compounds inhibit the processing of amyloid precursor protein and, more specifically, inhibit the production of Aβ-peptide, thereby acting to prevent the formation of neurological deposits of amyloid protein. More particularly, the present invention relates to the treatment of neurological disorders related to β-amyloid production such as Alzheimer's disease and Down's Syndrome.

### BACKGROUND OF THE INVENTION

Alzheimer's disease (AD) is a degenerative brain disorder characterized clinically by progressive loss of memory, temporal and local orientation, cognition, reasoning, judgment and emotional stability. AD is a common cause of progressive dementia in humans and is one of the major causes of death in the United States. AD has been observed in all races and ethnic groups worldwide, and is a major present and future health problem. No treatment that effectively prevents AD or reverses the clinical symptoms and underlying pathophysiology is currently available (for review, Dennis J. Selkoe; Cell Biology of the amyloid (beta)-protein precursor and the mechanism of Alzheimer's disease, Annu Rev Cell Biol, 1994, 10: 373-403).

Histopathological examination of brain tissue derived upon autopsy or from neurosurgical specimens in effected individuals revealed the occurrence of amyloid plaques and neurofibrillar tangles in the cerebral cortex of such patients. Similar alterations were observed in patients with Trisomy 21 (Down's syndrome), and hereditary cerebral hemorrhage with amyloidosis of the Dutch-type. Neurofibrillar tangles are nonmembrane-bound bundles of abnormal proteinaceous filaments and biochemical and immunochemical studies led to the conclusion that their principle protein subunit is an altered phosphorylated form of the tau protein (reviewed in Selkoe, 1994).

Biochemical and immunological studies revealed that the dominant proteinaceous component of the amyloid plaque is an approximately 4.2 kilodalton (kD) protein of about 39 to 43 amino acids. This protein was designated Aβ, β-amyloid peptide, and sometimes β/A4; referred to herein as Aβ. In addition to deposition of Aβ in amyloid plaques, Aβ is also found in the walls of meningeal and parenchymal arterioles, small arteries, capillaries, and sometimes, venules. Aβ was first purified, and a partial amino acid reported, in 1984 (Glenner and Wong, Biochem. Biophys. Res. Commun. 120: 885-890). The isolation and sequence data for the first 28 amino acids are described in U.S. Pat. No. 4,666,829.

Compelling evidence accumulated during the last decade revealed that Aβ is an internal polypeptide derived from a type 1 integral membrane protein, termed β amyloid precursor protein (APP). β APP is normally produced by many cells both in vivo and in cultured cells, derived from various animals and humans. Aβ is derived from cleavage of β APP by as yet unknown enzyme (protease) system(s), collectively termed secretases.

The existence of at least four proteolytic activities has been postulated. They include β secretase(s), generating the N-terminus of Aβ, a secretase(s) cleaving around the 16/17 peptide bond in Aβ, and γ secretases, generating C-terminal Aβ fragments ending at position 38, 39, 40, 42, and 43 or generating C-terminal extended precursors which are subsequently truncated to the above polypeptides.

Several lines of evidence suggest that abnormal accumulation of Aβ plays a key role in the pathogenesis of AD. Firstly, Aβ is the major protein found in amyloid plaques. Secondly, Aβ is neurotoxic and may be causally related to neuronal death observed in AD patients. Thirdly, missense DNA mutations at position 717 in the 770 isoform of β APP can be found in effected members but not unaffected members of several families with a genetically determined (familiar) form of AD. In addition, several other β APP mutations have been described in familiar forms of AD. Fourthly, similar neuropathological changes have been observed in transgenic animals overexpressing mutant forms of human β APP. Fifthly, individuals with Down's syndrome have an increased gene dosage of β APP and develop early-onset AD. Taken together, these observations strongly suggest that Aβ depositions may be causally related to the AD.

It is hypothesized that inhibiting the production of Aβ will prevent and reduce neurological degeneration, by controlling the formation of amyloid plaques, reducing neurotoxicity and, generally, mediating the pathology associated with Aβ production. One method of treatment methods would therefore be based on drugs that inhibit the formation of Aβ in vivo.

Methods of treatment could target the formation of Aβ through the enzymes involved in the proteolytic processing of β amyloid precursor protein. Compounds that inhibit β or γ secretase activity, either directly or indirectly, could control the production of Aβ. Advantageously, compounds that specifically target γ secretases, could control the production of Aβ. Such inhibition of β or γ secretases could thereby reduce production of Aβ, which, thereby, could reduce or prevent the neurological disorders associated with Aβ protein.

PCT publication number WO 96/29313 discloses the general formula:

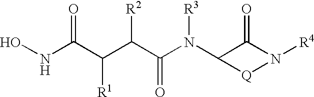

covering metalloprotease inhibiting compounds useful for the treatment of diseases associated with excess and/or unwanted matrix metalloprotease activity, particularly collagenase and or stromelysin activity.

Compounds of general formula:

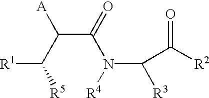

are disclosed in PCT publication number WO 95/22966 relating to matrix metalloprotease inhibitors. The compounds of the invention are useful for the treatment of conditions associated with the destruction of cartilage, including corneal ulceration, osteoporosis, periodontitis and cancer.

European Patent Application number EP 0652009A1 relates to the general formula:

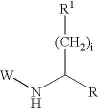

and discloses compounds that are protease inhibitors that inhibit Aβ production.

U.S. Pat. No. 5,703,129 discloses the general formula:

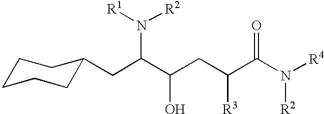

which covers 5-amino-6-cyclohexyl-4-hydroxy-hexanamide derivatives that inhibit Aβ production and are useful in the treatment of Alzheimer's disease.

Thus there remains a need to develop compounds which are useful as inhibitors of the production of Aβ protein or pharmaceutically acceptable salts or prodrugs thereof, for the treatment of degenerative neurological disorders, such as Alzheimer's disease.

None of the above references teaches or suggests the compounds of the present invention which are described in detail below.

### SUMMARY OF THE INVENTION

One object of the present invention is to provide novel compounds which are useful as inhibitors of the production of Aβ protein or pharmaceutically acceptable salts or prodrugs thereof.

It is another object of the present invention to provide pharmaceutical compositions comprising a pharmaceutically acceptable carrier and a therapeutically effective amount of at least one of the compounds of the present invention or a pharmaceutically acceptable salt or prodrug form thereof.

It is another object of the present invention to provide a method for treating degenerative neurological disorders comprising administering to a host in need of such treatment a therapeutically effective amount of at least one of the compounds of the present invention or a pharmaceutically acceptable salt or prodrug form thereof.

These and other objects, which will become apparent during the following detailed description, have been achieved by the inventors, discovery that compounds of Formula (I):

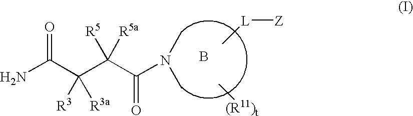

or pharmaceutically acceptable salt or prodrug forms thereof, wherein R³, R^{3a}, R⁵, R^{5a}, R¹¹, t, B, L, and Z are defined below, are effective inhibitors of the production of Aβ protein.

### DETAILED DESCRIPTION OF PREFERRED EMBODIMENTS

Thus, in a first embodiment, the present invention provides a novel compound of Formula (I):

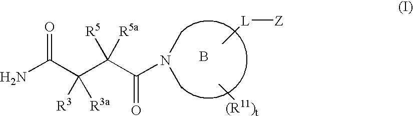

or a pharmaceutically acceptable salt or prodrug thereof, wherein:

R³ is —(CR⁷R^{7a})ₙ—R⁴,

—(CR⁷R^{7a})ₙ—S—(CR⁷R^{7a})ₘ—R⁴,

—(CR⁷R^{7a})ₙ—O—(CR⁷R^{7a})ₘ—R⁴,

—(CR⁷R^{7a})ₙ—N(R^{7b})—(CR⁷R^{7a})ₘ—R⁴,

—(CR⁷R^{7a})ₙ—S(═O)—(CR⁷R^{7a})ₘ—R⁴,

—(CR⁷R^{7a})ₙ—S(═O)₂—(CR⁷R^{7a})ₘ—R⁴,

—(CR⁷R^{7a})ₙ—C(═O)—(CR⁷R^{7a})ₘ—R⁴,

—(CR⁷R^{7a})ₙ—N(R^{7b})C(═O)—(CR⁷R^{7a})ₘ—R⁴,

—(CR⁷R^{7a})ₙ—C(═O)N(R^{7b})—(CR⁷R^{7a})ₘ—R⁴,

—(CR⁷R^{7a})ₙ—N(R^{7b})S(═O)₂—(CR⁷R^{7a})ₘ—R⁴, or

—(CR⁷R^{7a})ₙ—S(═O)₂N(R^{7b})—(CR⁷R^{7a})ₘ—R⁴;

provided R³ is not hydrogen when R⁵ is hydrogen;

n is 0, 1, 2, or 3;

m is 0, 1, 2, or 3;

R^{3a} is H, OH, C₁-C₄ alkyl, C₁-C₄ alkoxy, C₂-C₄ alkenyl, or C₂-C₄ alkenyloxy;

alternatively, R³ and R^{3a}, and the carbon to which they are attached, may be combined to form a 3-8 membered cycloalkyl moiety substituted with 0-2 R^{4b}; provided that R⁵ and R^{5a} are not combined to form a 3-8 membered cycloalkyl moiety;

R⁴ is H, OH, OR^{14a},

C₁-C₆ alkyl substituted with 0-3 R^{4a},

C₂-C₆ alkenyl substituted with 0-3 R^{4a},

C₂-C₆ alkynyl substituted with 0-3 R^{4a},

C₃-C₁₀ carbocycle substituted with 0-3 R^{4b},

C₆-C₁₀ aryl substituted with 0-3 R^{4b}, or

5 to 10 membered heterocycle substituted with 0-3 R^{4b};

R^{4a}, at each occurrence, is independently selected from: H,

F, Cl, Br, I, CF₃,

C₃-C₁₀ carbocycle substituted with 0-3 R^{4b},

C₆-C₁₀ aryl substituted with 0-3 R^{4b}, or

5 to 10 membered heterocycle substituted with 0-3 R^{4b};

R^{4b}, at each occurrence, is independently selected from:

H, OH, Cl, F, Br, I, CN, NO₂, NR¹⁵R¹⁶, CF₃, acetyl,

SCH₃, S(═O)CH₃, S(═O)₂CH₃, C₁-C₆ alkyl, C₁-C₄ alkoxy,

C₁-C₄ haloalkyl, C₁-C₄ haloalkoxy, and C₁-C₄

halothioalkoxy;

R⁵ is H, OR¹⁴;

C₁-C₆ alkyl substituted with 0-3 R^{5b};

C₁-C₆ alkoxy substituted with 0-3 R^{5b};

C₂-C₆ alkenyl substituted with 0-3 R^{5b};

C₂-C₆ alkynyl substituted with 0-3 R^{5b};

C₃-C₁₀ carbocycle substituted with 0-3 R^{5c};

C₆-C₁₀ aryl substituted with 0-3 R^{5c}; or

5 to 10 membered heterocycle substituted with 0-3 R^{5c};

provided R⁵ is not hydrogen when R³ is hydrogen;

R^{5a} is H, OH, C₁-C₄ alkyl, C₁-C₄ alkoxy, C₂-C₄ alkenyl, or C₂-C₄ alkenyloxy;

R^{5b}, at each occurrence, is independently selected from:

H, C₁-C₆ alkyl, CF₃, OR¹⁴, Cl, F, Br, I, ═O, CN, NO₂, NR¹⁵R¹⁶;

C₃-C₁₀ carbocycle substituted with 0-3 R^{5c};

C₆-C₁₀ aryl substituted with 0-3 R^{5c}; or

5 to 10 membered heterocycle substituted with 0-3 R^{5c};

R^{5c}, at each occurrence, is independently selected from: H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, C₁-C₄ haloalkoxy, and C₁-C₄ halothioalkoxy;

alternatively, R⁵ and R^{5a}, and the carbon to which they are attached, may be combined to form a 3-8 membered cycloalkyl moiety substituted with 0-2 R^{5b}; provided that R³ and R^{3a} are not combined to form a 3-8 membered cycloalkyl moiety;

R⁷, at each occurrence, is independently selected from: H, OH, Cl, F, Br, I, CN, NO₂, CF₃, and C₁-C₄ alkyl;

R^{7a}, at each occurrence, is independently selected from: H, OH, Cl, F, Br, I, CN, NO₂, CF₃, aryl and C₁-C₄ alkyl;

R^{7b} is independently selected from H and C₁-C₄ alkyl;

L is a bond, C₁-C₄ alkyl, C₂-C₄ alkenyl, C₂-C₄ alkynyl, —(CH₂)ₚ—O—(CH₂)_{q}—, or —(CH₂)ₚ—NR¹⁰—(CH₂)_{q}—;

p is 0, 1, 2, or 3;

q is 0, 1, 2, or 3;

Z is C₃-C₁₀ carbocycle substituted with 0-2 R^{12b};

C₆-C₁₀ aryl substituted with 0-4 R^{12b}; and

5 to 10 membered heterocycle substituted with 0-5 R^{12b}, wherein the heterocycle contains 1, 2, 3 or 4 heteroatoms selected from N, O and S;

R^{12b}, at each occurrence, is independently selected from: H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, C₁-C₄ haloalkoxy, C₁-C₄ halothioalkoxy, aryl substituted with 0-4 R^{12c};

R^{12c}, at each occurrence, is independently selected from: H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, C₁-C₄ haloalkoxy, and C₁-C₄ halothioalkoxy;

B is a 4 to 8 membered amino-heterocyclic ring, comprising one N atom, 3 to 7 carbon atoms, and optionally, an additional heteroatom selected from —O—, —S—, —S(═O)—, —S(═O)₂—, and —N(R^{LZ})—;

wherein the amino-heterocyclic ring is saturated or partially saturated; and

wherein R^{LZ} is either R¹⁰ or the substituent —L—Z;

R¹⁰ is H, C(═O)R¹⁷, C(═O)OR¹⁷, —(C₁-C₃ alkyl)—C(═O)OR¹⁷, C(═O)NR¹⁸R¹⁹, S(═O)₂NR¹⁸R¹⁹, S(═O)₂R¹⁷;

C₁-C₆ alkyl substituted with 0-2 R^{10a};

C₆-C₁₀ aryl substituted with 0-4 R^{10b};

C₃-C₁₀ carbocycle substituted with 0-3 R^{10b}; or

5 to 10 membered heterocycle optionally substituted with 0-3 R^{10b};

R^{10a}, at each occurrence, is independently selected from: H, C₁-C₆ alkyl, OR¹⁴, Cl, F, Br, I, ═O, CN, NO₂, NR¹⁵R¹⁶, CF₃, or aryl substituted with 0-4 R^{10b};

R^{10b}, at each occurrence, is independently selected from: H, OH, C₁-C₆ alkyl, C₁-C₄ alkoxy, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, C₁-C₄ haloalkoxy, and C₁-C₄ halothioalkoxy;

R¹¹, at each occurrence, is independently selected from:

C₁-C₄ alkoxy, Cl, F, Br, I, —OH, CN, NO₂, NR¹⁸R¹⁹, C(═O)R¹⁷, C(═O)OR¹⁷, C(═O)NR¹⁸R¹⁹, S(═O)₂NR¹⁸R¹⁹, CF₃;

C₁-C₆ alkyl substituted with 0-1 R^{11a};

C₆-C₁₀ aryl substituted with 0-3 R^{11b};

C₃-C₁₀ carbocycle substituted with 0-3 R^{11b}; or

5 to 10 membered heterocycle substituted with 0-3 R^{11b};

alternatively, two R¹¹ substituents on the same or adjacent carbon atoms may be combined to form a C₃-C₆ carbocycle or a benzo fused radical, wherein said carbocycle or benzo fused radical is substituted with 0-4 R¹³;

additionally, two R¹¹ substituents on adjacent atoms may be combined to form a 5 to 6 membered heteroaryl fused radical, wherein said 5 to 6 membered heteroaryl fused radical comprises 1 or 2 heteroatoms selected from N, O, and S; wherein said 5 to 6 membered heteroaryl fused radical is substituted with 0-3 R¹³;

R^{11a}, at each occurrence, is independently selected from: H, C₁-C₆ alkyl, OR¹⁴, Cl, F, Br, I, ═O, CN, NO₂, NR¹⁵R¹⁶, CF₃, or phenyl substituted with 0-3 R^{11b};

R^{11b}, at each occurrence, is independently selected from: H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, C₁-C₄ haloalkoxy, and C₁-C₄ halothioalkoxy;

t is 0, 1, 2 or 3;

R¹³, at each occurrence, is independently selected from: H, OH, C₁-C₆ alkyl, C₁-C₄ alkoxy, Cl, F, Br, I, CN, NO₂, NR¹⁵R¹⁶, and CF₃;

R¹⁴, at each occurrence, is independently selected from: H, phenyl, benzyl, C₁-C₆ alkyl, or C₂-C₆ alkoxyalkyl;

R^{14a} is H, phenyl, benzyl, or C₁-C₄ alkyl;

R¹⁵, at each occurrence, is independently selected from: H, C₁-C₆ alkyl, benzyl, phenethyl, —C(═O)—(C₁-C₆ alkyl), —S(═O)₂—(C₁-C₆ alkyl), and aryl;

R¹⁶, at each occurrence, is independently selected from: H, OH, C₁-C₆ alkyl, benzyl, phenethyl, —C(═O)—(C₁-C₆ alkyl) and —S(═O)₂—(C₁-C₆ alkyl);

alternatively, R¹⁵ and R¹⁶ on the same N atom may be combined to form a 5 to 6 membered heterocyclic fused radical, wherein said 5 to 6 membered heterocyclic fused radical comprises 1 or 2 heteroatoms selected from N and O;

R¹⁷ is H, aryl, aryl-CH₂—, C₁-C₆ alkyl, or C₂-C₆ alkoxyalkyl;

R¹⁸, at each occurrence, is independently selected from: H, C₁-C₆ alkyl, benzyl, phenethyl, —C(═O)—(C₁-C₆ alkyl) and —S(═O)₂—(C₁-C₆ alkyl);

R¹⁹, at each occurrence, is independently selected from: H, OH, C₁-C₆ alkyl, phenyl, benzyl, phenethyl, —C(═O)—(C₁-C₆ alkyl) —S(═O)₂—(C₁-C₆ alkyl); and

alternatively, R¹⁸ and R¹⁹ on the same N atom may be combined to form a 5 to 6 membered heterocyclic fused radical, wherein said 5 to 6 membered heterocyclic fused radical comprises 1 or 2 heteroatoms selected from N and O.

[2] In a preferred embodiment the present provides a compound of Formula (I) wherein:

R³ is —(CR⁷R^{7a})ₙ—R⁴,

—(CR⁷R^{7a})ₙ—S—(CR⁷R^{7a})ₘ—R⁴,

—(CR⁷R^{7a})ₙ—O—(CR⁷R^{7a})ₘ—R⁴,

—(CR⁷R^{7a})ₙ—N(R^{7b})—(CR⁷R^{7a})ₘ—R⁴,

—(CR⁷R^{7a})ₙ—S(═O)—(CR⁷R^{7a})ₘ—R⁴,

—(CR⁷R^{7a})ₙ—S(═O)₂—(CR⁷R^{7a})ₘ—R⁴,

—(CR⁷R^{7a})ₙ—C(═O)—(CR⁷R^{7a})ₘ—R⁴,

—(CR⁷R^{7a})ₙ—NHC(═O)—(CR⁷R^{7a})ₘ—R⁴,

—(CR⁷R^{7a})ₙ—C(═O)NH—(CR⁷R^{7a})ₘ—R⁴,

—(CR⁷R^{7a})ₙ—NHS(═O)₂—(CR⁷R^{7a})ₘ—R⁴, or

—(CR⁷R^{7a})ₙ—S(═O)₂NH—(CR⁷R^{7a})ₘ—R⁴;

provided R³ is not hydrogen when R⁵ is hydrogen;

n is 0, 1, 2, or 3;

m is 0, 1, 2, or 3;

R^{3a} is H, OH, C₁-C₄ alkyl, C₁-C₄ alkoxy, C₂-C₄ alkenyl, or C₂-C₄ alkenyloxy;

alternatively, R³ and R^{3a}, and the carbon to which they are attached, may be combined to form a 3-8 membered cycloalkyl moiety substituted with 0-1 R^{4b}; provided that R⁵ and R^{5a} are not combined to form a 3-8 membered cycloalkyl moiety;

R⁴ is H, OH, OR^{14a},

C₁-C₆ alkyl substituted with 0-3 R^{4a},

C₂-C₆ alkenyl substituted with 0-3 R^{4a},

C₂-C₆ alkynyl substituted with 0-3 R^{4a},

C₃-C₁₀ carbocycle substituted with 0-3 R^{4b},

C₆-C₁₀ aryl substituted with 0-3 R^{4b}, or

5 to 10 membered heterocycle substituted with 0-3 R^{4b};

R^{4a}, at each occurrence, is independently selected from: H,

F, Cl, Br, I, CF₃,

C₃-C₁₀ carbocycle substituted with 0-3 R^{4b},

C₆-C₁₀ aryl substituted with 0-3 R^{4b}, or

5 to 10 membered heterocycle substituted with 0-3 R^{4b};

R^{4b}, at each occurrence, is independently selected from: H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, and C₁-C₄ halothioalkoxy;

R⁵ is H, OR¹⁴;

C₁-C₆ alkyl substituted with 0-3 R^{5b};

C₁-C₆ alkoxy substituted with 0-3 R^{5b};

C₂-C₆ alkenyl substituted with 0-3 R^{5b};

C₂-C₆ alkynyl substituted with 0-3 R^{5b};

C₃-C₁₀ carbocycle substituted with 0-3 R^{5c};

C₆-C₁₀ aryl substituted with 0-3 R^{5c}; or

5 to 10 membered heterocycle substituted with 0-3 R^{5c};

provided R⁵ is not hydrogen when R³ is hydrogen;

R^{5a} is H, OH, methyl, ethyl, propyl, butyl, methoxy, ethoxy, propoxy, butoxy, or allyl;

R^{5b}, at each occurrence, is independently selected from:

H, C₁-C₆ alkyl, CF₃, OR¹⁴, Cl, F, Br, I, ═O, CN, NO₂, NR¹⁵R¹⁶;

C₃-C₁₀ carbocycle substituted with 0-3 R^{5c};

C₆-C₁₀ aryl substituted with 0-3 R^{5c}; or

5 to 10 membered heterocycle substituted with 0-3 R^{5c};

R^{5c}, at each occurrence, is independently selected from: H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, and C₁-C₄ haloalkoxy;

alternatively, R⁵ and R^{5a}, and the carbon to which they are attached, may be combined to form a 3-8 membered cycloalkyl moiety substituted with 0-1 R^{5b}; provided that R³ and R^{3a} are not combined to form a 3-8 membered cycloalkyl moiety;

R⁷, at each occurrence, is independently selected from: H, OH, Cl, F, Br, I, CN, NO₂, CF₃, and C₁-C₄ alkyl;

R^{7a}, at each occurrence, is independently selected from: H, OH, Cl, F, Br, I, CN, NO₂, CF₃, aryl and C₁-C₄ alkyl;

R^{7b} is independently selected from H and C₁-C₄ alkyl;

L is a bond, C₁-C₄ alkyl, C₂-C₄ alkenyl, C₂-C₄ alkynyl, —(CH₂)ₚ—O—(CH₂)_{q}—, or —(CH₂)ₚ—NR¹⁰—(CH₂)_{q}—;

p is 0, 1, 2, or 3;

q is 0, 1, 2, or 3;

Z is C₃-C₁₀ carbocycle substituted with 0-2 R^{12b};

C₆-C₁₀ aryl substituted with 0-4 R^{12b}; and

5 to 10 membered heterocycle substituted with 0-5 R^{12b}, wherein the heterocycle contains 1, 2, 3 or 4 heteroatoms selected from N, O and S;

R^{12b}, at each occurrence, is independently selected from: H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, C₁-C₄ haloalkoxy, aryl substituted with 0-4 R^{12c};

R^{12c}, at each occurrence, is independently selected from: H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, and C₁-C₄ haloalkoxy;

B is a 4 to 8 membered amino-heterocyclic ring, comprising one N atom, 3 to 7 carbon atoms, and optionally, an additional heteroatom selected from —O—, —S—, —S(═O)—, —S(═O)₂—, and —N(R^{LZ})—;

wherein the amino-heterocyclic ring is saturated or partially saturated; and

wherein R^{LZ} is either R¹⁰ or the substituent —L—Z;

R¹⁰ is H, C(═O)R¹⁷, C(═O)OR¹⁷, —(C₁-C₃ alkyl)—C(═O)OR¹⁷, C(═O)NR¹⁸R¹⁹, S(═O)₂NR¹⁸R¹⁹, S(═O)₂R¹⁷;

C₁-C₆ alkyl substituted with 0-2 R^{10a};

C₆-C₁₀ aryl substituted with 0-4 R^{10b};

C₃-C₁₀ carbocycle substituted with 0-3 R^{10b}; or

5 to 10 membered heterocycle optionally substituted with 0-3 R^{10b};

R^{10a}, at each occurrence, is independently selected from: H, C₁-C₆ alkyl, OR¹⁴, Cl, F, Br, I, ═O, CN, NO₂, NR¹⁵R¹⁶, CF₃, or aryl substituted with 0-4 R^{10b};

R^{10b}, at each occurrence, is independently selected from: H, OH, C₁-C₆ alkyl, C₁-C₄ alkoxy, Cl, F, Br, I, CN, NO₂, NR¹⁵R¹⁶, CF₃;

R¹¹, at each occurrence, is independently selected from:

C₁-C₄ alkoxy, Cl, F, Br, I, —OH, CN, NO₂, NR¹⁸R¹⁹, C(═O)R¹⁷, C(═O)OR¹⁷, C(═O)NR¹⁸R¹⁹, S(═O)₂NR¹⁸R¹⁹, CF₃;

C₁-C₆ alkyl substituted with 0-1 R^{11a};

C₆-C₁₀ aryl substituted with 0-3 R^{11b};

C₃-C₁₀ carbocycle substituted with 0-3 R^{11b}; or

5 to 10 membered heterocycle substituted with 0-3 R^{11b};

alternatively, two R¹¹ substituents on the same or adjacent carbon atoms may be combined to form a C₃-C₆ carbocycle or a benzo fused radical wherein said benzo benzo fused radical is substituted with 0-4 R¹³;

additionally, two R¹¹ substituents on adjacent atoms may be combined to form a 5 to 6 membered heteroaryl fused radical, wherein said 5 to 6 membered heteroaryl fused radical comprises 1 or 2 heteroatoms selected from N, O, and S; wherein said 5 to 6 membered heteroaryl fused radical is substituted with 0-3 R¹³;

R^{11a}, at each occurrence, is independently selected from: H, C₁-C₆ alkyl, OR¹⁴, Cl, F, Br, I, ═O, CN, NO₂, NR¹⁵R¹⁶, CF₃, or phenyl substituted with 0-3 R^{11b};

R^{11b}, at each occurrence, is independently selected from: H, OH, Cl, F, Br, I, CN, NO₂, NR¹⁵R¹⁶, CF₃, C₁-C₆ alkyl, C₁-C₄ alkoxy, C₁-C₄ haloalkoxy, and C₁-C₄ haloalkoxy;

t is 0, 1, 2 or 3;

R¹³, at each occurrence, is independently selected from: H, OH, C₁-C₆ alkyl, C₁-C₄ alkoxy, Cl, F, Br, I, CN, NO₂, NR¹⁵R¹⁶, and CF₃;

R¹⁴ is H, phenyl, benzyl, C₁-C₆ alkyl, or C₂-C₆ alkoxyalkyl;

R^{14a} is H, phenyl, benzyl, or C₁-C₄ alkyl;

R¹⁵, at each occurrence, is independently selected from: H, C₁-C₆ alkyl, benzyl, phenethyl, —C(═O)—(C₁-C₆ alkyl) and —S(═O)₂—(C₁-C₆ alkyl), and aryl;

R¹⁶, at each occurrence, is independently selected from: H, OH, C₁-C₆ alkyl, benzyl, phenethyl, —C(═O)—(C₁-C₆ alkyl) and —S(═O)₂—(C₁-C₆ alkyl), and phenyl substituted with 0-3 R¹³;

alternatively, R¹⁵ and R¹⁶ on the same N atom may be combined to form a 5 to 6 membered heterocyclic fused radical, wherein said 5 to 6 membered heterocyclic fused radical comprises 1 or 2 heteroatoms selected from N and O;

R¹⁷ is H, aryl, (aryl)CH₂—, C₁-C₆ alkyl, or C₂-C₆ alkoxyalkyl;

R¹⁸, at each occurrence, is independently selected from: H, C₁-C₆ alkyl, benzyl, phenethyl, —C(═O)—(C₁-C₆ alkyl) and —S(═O)₂—(C₁-C₆ alkyl);

R¹⁹, at each occurrence, is independently selected from: H, OH, C₁-C₆ alkyl, phenyl, benzyl, phenethyl, —C(═O)—(C₁-C₆ alkyl) and —S(═O)₂—(C₁-C₆ alkyl); and

alternatively, R¹⁸ and R¹⁹ on the same N atom may be combined to form a 5 to 6 membered heterocyclic fused radical, wherein said 5 to 6 membered heterocyclic fused radical comprises 1 or 2 heteroatoms selected from N and O.

[3] In a another preferred embodiment the present invention provides a compound of Formula (I) wherein:

R³ is —(CHR⁷)ₙ—R⁴,

—(CHR⁷)ₙ—S—(CHR⁷)ₘ—R⁴,

—(CHR⁷)ₙ—O—(CHR⁷)ₘ—R⁴, or

—(CHR⁷)ₙ—N(R^{7b})—(CHR⁷)ₘ—R⁴;

provided R³ is not hydrogen when R⁵ is hydrogen;

n is 0, 1, or 2;

m is 0, 1, or 2;

R^{3a} is H;

alternatively, R³ and R^{3a}, and the carbon to which they are attached, may be combined to form a cyclopropyl, cyclobutyl, cyclopentyl, or cyclohexyl moiety; provided that R⁵ and R^{5a}are not combined to form a cycloalkyl moiety;

R⁴ is H, OH, OR^{14a},

C₁-C₄ alkyl substituted with 0-2 R^{4a},

C₂-C₄ alkenyl substituted with 0-2 R^{4a},

C₂-C₄ alkynyl substituted with 0-2 R^{4a},

C₃-C₆ cycloalkyl substituted with 0-3 R^{4b},

phenyl substituted with 0-3 R^{4b}, or

5 to 6 membered heterocycle substituted with 0-3 R^{4b};

R^{4a}, at each occurrence, is independently selected from: H,

F, Cl, Br, I CF₃,

C₃-C₁₀ carbocycle substituted with 0-3 R^{4b},

phenyl substituted with 0-3 R^{4b}, or

5 to 6 membered heterocycle substituted with 0-3 R^{4b};

R^{4b}, at each occurrence, is independently selected from: H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, and C₁-C₄ haloalkoxy;

R⁵ is H, OR¹⁴;

C₁-C₆ alkyl substituted with 0-3 R^{5b};

C₂-C₆ alkenyl substituted with 0-3 R^{5b};

C₂-C₆ alkynyl substituted with 0-3 R^{5b};

C₃-C₁₀ carbocycle substituted with 0-3 R^{5c};

C₆-C₁₀ aryl substituted with 0-3 R^{5c}; or

5 to 10 membered heterocycle substituted with 0-3R^{5c};

provided R⁵ is not hydrogen when R³ is hydrogen;

R^{5a} is H;

R^{5b}, at each occurrence, is independently selected from:

H, C₁-C₆ alkyl, CF₃, OR¹⁴, Cl, F, Br, I, ═O, CN, NO₂, NR¹⁵R¹⁶;

C₃-C₁₀ carbocycle substituted with 0-3 R^{5c};

C₆-C₁₀ aryl substituted with 0-3 R^{5c}; or

5 to 10 membered heterocycle substituted with 0-3 R^{5c};

R^{5c}, at each occurrence, is independently selected from: H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, and C₁-C₄ haloalkoxy;

alternatively, R⁵ and R^{5a}, and the carbon to which they are attached, may be combined to form a cyclopropyl, cyclobutyl, cyclopentyl, or cyclohexyl moiety; provided that R³ and R^{3a} are not combined to form a cycloalkyl moiety;

R⁷, at each occurrence, is independently selected from: H, OH, Cl, F, Br, I, CN, NO₂, CF₃, and C₁-C₄ alkyl;

R^{7b} is independently selected from: H, methyl, ethyl, propyl, and butyl;

L is a bond, —CH₂—, —CH₂CH₂—, —CH₂CH₂CH₂—, —CH₂CH═CH₂, —(CH₂)ₚ—O—(CH₂)_{q}—, or —(CH₂)ₚ—NR¹⁰—(CH₂)_{q}—;

p is 0, 1, 2, or 3;

q is 0, 1, 2, or 3;

Z is C₃-C₁₀ carbocycle substituted with 0-2 R^{12b};

C₆-C₁₀ aryl substituted with 0-4 R^{12b}; and

5 to 10 membered heterocycle substituted with 0-5 R^{12b}, wherein the heterocycle contains 1, 2, 3 or 4 heteroatoms selected from N, O and S;

R^{12b}, at each occurrence, is independently selected from: H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, C₁-C₄ haloalkoxy, phenyl substituted with 0-3 R^{12c};

R^{12c}, at each occurrence, is independently selected from: H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, and C₁-C₄ haloalkoxy;

B is a 5, 6, or 7 membered amino-heterocyclic ring, comprising one N atom, 3 to 6 carbon atoms, and optionally, an additional heteroatom —N(R^{LZ})—;

wherein the amino-heterocyclic ring is saturated or partially saturated; and

wherein R^{LZ} is either R¹⁰ or the substituent —L—Z;

R¹⁰ is H, C(═O)R¹⁷, C(═O)OR¹⁷, —(C₁-C₃ alkyl)—C(═O)OR¹⁷,

C(═O)NR¹⁸R¹⁹, S(═O)₂NR¹⁸R¹⁹, S(═O)₂R¹⁷;

C₁-C₆ alkyl substituted with 0-1 R^{10a};

C₆-C₁₀ aryl substituted with 0-4 R^{10b};

C₃-C₁₀ carbocycle substituted with 0-3 R^{10b}; or

5 to 10 membered heterocycle optionally substituted with 0-3 R^{10b};

R^{10a}, at each occurrence, is independently selected from: H, C₁-C₆ alkyl, OR¹⁴, Cl, F, Br, I, ═O, CN, NO₂, NR¹⁵R¹⁶, CF₃, or phenyl substituted with 0-4 R^{10b};

R^{10b}, at each occurrence, is independently selected from H, OH, C₁-C₆ alkyl, C₁-C₄ alkoxy, Cl, F, Br, I, CN, NO₂, NR¹⁵R¹⁶, or CF₃;

R¹¹, at each occurrence, is independently selected from:

C₁-C₄ alkoxy, Cl, F, NR¹⁸R¹⁹, C(═O)R¹⁷, C(═O)OR¹⁷, C(═O)NR¹⁸R¹⁹, S(═O)₂NR¹⁸R¹⁹, CF₃;

C₁-C₆ alkyl substituted with 0-1 R^{11a};

C₆-C₁₀ aryl substituted with 0-3 R^{11b};

C₃-C₁₀ carbocycle substituted with 0-3 R^{11b}; or

5 to 10 membered heterocycle substituted with 0-3 R^{11b};

alternatively, two R¹¹ substituents on the same or adjacent carbon atoms may be combined to form a C₃-C₆ carbocycle or a benzo fused radical wherein said benzo fused radical is substituted with 0-4 R¹³;

additionally, two R¹¹ substituents on adjacent atoms may be combined to form a 5 to 6 membered heteroaryl fused radical, wherein said 5 to 6 membered heteroaryl fused radical comprises 1 or 2 heteroatoms selected from N, O, and S; wherein said 5 to 6 membered heteroaryl fused radical is substituted with 0-3 R¹³;

R^{11a}, at each occurrence, is independently selected from: H, C₁-C₆ alkyl, OR¹⁴, Cl, F, Br, I, ═O, CN, NO₂, NR¹⁵R¹⁶, CF₃, or phenyl substituted with 0-3 R^{11b};

R^{11b}, at each occurrence, is independently selected from: H, OH, Cl, F, Br, I, CN, NO₂, NR¹⁵R¹⁶, CF₃, C₁-C₆ alkyl, C₁-C₄ alkoxy, C₁-C₄ haloalkyl, and C₁-C₄ haloalkoxy;

t is 0, 1, 2 or 3;

R¹³, at each occurrence, is independently selected from: H, OH, C₁-C₆ alkyl, C₁-C₄ alkoxy, Cl, F, Br, I, CN, NO₂, NR¹⁵R¹⁶, and CF₃;

R¹⁴ is H, phenyl, benzyl, C₁-C₆ alkyl, or C₂-C₆ alkoxyalkyl;

R^{14a} is H, phenyl, benzyl, or C₁-C₄ alkyl;

R¹⁵, at each occurrence, is independently selected from: H, C₁-C₆ alkyl, benzyl, phenethyl, —C(═O)—(C₁-C₆ alkyl), —S(═O)₂—(C₁-C₆ alkyl), and aryl;

R¹⁶, at each occurrence, is independently selected from: H, OH, C₁-C₆ alkyl, benzyl, phenethyl, —C(═O)—(C₁-C₆ alkyl) and —S(═O)₂—(C₁-C₆ alkyl);

alternatively, R¹⁵ and R¹⁶ on the same N atom may be combined to form a 5 to 6 membered heterocyclic fused radical, wherein said 5 to 6 membered heterocyclic is selected from pyrrolidonyl, piperidonyl, piperazinyl, and morpholinyl;

R¹⁷ is H, aryl, (aryl)CH₂—, C₁-C₆ alkyl, or C₂-C₆ alkoxyalkyl;

R¹⁸, at each occurrence, is independently selected from: H, C₁-C₆ alkyl, benzyl, phenethyl, —C(═O)—(C₁-C₆ alkyl) and —S(═O)₂—(C₁-C₆ alkyl);

R¹⁹, at each occurrence, is independently selected from: H, OH, C₁-C₆ alkyl, phenyl, benzyl, phenethyl, —C(═O)—(C₁-C₆ alkyl) and —S(═O)₂—(C₁-C₆ alkyl); and

alternatively, R¹⁸ and R¹⁹ on the same N atom may be combined to form a 5 to 6 membered heterocyclic fused radical, wherein said 5 to 6 membered heterocyclic is selected from pyrrolidonyl, piperidonyl, piperazinyl, and morpholinyl.

[4] In a another preferred embodiment the present invention provides a compound of Formula (Ic):

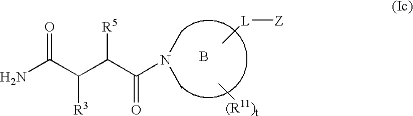

or a pharmaceutically acceptable salt or prodrug thereof, wherein:

R³ is C₁-C₄ alkyl substituted with 0-2 R^{4a},

C₂-C₄ alkenyl substituted with 0-2 R^{4a}, or

C₂-C₄ alkynyl substituted with 0-1 R^{4a};

R^{4a}, at each occurrence, is independently selected from: H,

F, Cl, CF₃,

C₃-C₆ cycloalkyl substituted with 0-3 R^{4b},

phenyl substituted with 0-3 R^{4b}, or

5 to 6 membered heterocycle substituted with 0-3 R^{4b};

R^{4b}, at each occurrence, is independently selected from: H, OH, Cl, F, Br, I, CN, NO₂, NR¹⁵R¹⁶, CF₃, acetyl, SCH₃, S(═O)CH₃, S(═O)₂CH₃, C₁-C₄ alkyl, C₁-C₃ alkoxy, C₁-C₂ haloalkyl, and C₁-C₂ haloalkoxy;

R⁵ is C₁-C₆ alkyl substituted with 0-3 R^{5b};

C₂-C₆ alkenyl substituted with 0-2 R^{5b}; or

C₂-C₆ alkynyl substituted with 0-2 R^{5b};

R^{5b}, at each occurrence, is independently selected from:

H, methyl, ethyl, propyl, butyl, CF₃, OR¹⁴, ═O;

C₃-C₆ cycloalkyl substituted with 0-2 R^{5c};

phenyl substituted with 0-3 R^{5c}; or

5 to 6 membered heterocycle substituted with 0-2 R^{5c};

R^{5c}, at each occurrence, is independently selected from: H, OH, Cl, F, Br, I, CN, NO₂, NR¹⁵R¹⁶, CF₃, acetyl, SCH₃, S(═O)CH₃, S(═O)₂CH₃, C₁-C₄ alkyl, C₁-C₃ alkoxy, C₁-C₂ haloalkyl, and C₁-C₂ haloalkoxy;

L is a bond, —CH₂—, —CH₂CH₂—, —CH₂CH₂CH₂—, —CH₂CH═CH₂, —(CH₂)ₚ—O—(CH₂)_{q}—, or —(CH₂)ₚ—NR¹⁰—(CH₂)_{q}—;

p is 0, 1, 2, or 3;

q is 0, 1, or 2;

Z is C₃-C₁₀ carbocycle substituted with 0-2 R^{12b};

C₆-C₁₀ aryl substituted with 0-4 R^{12b}; and

5 to 10 membered heterocycle substituted with 0-5 R^{12b}, wherein the heterocycle contains 1, 2, 3 or 4 heteroatoms selected from N, O and S;

R^{12b}, at each occurrence, is independently selected from: H, OH, Cl, F, NR¹⁵R¹⁶, CF₃, acetyl, SCH₃, S(═O)CH₃, S(═O)₂CH₃, C₁-C₄ alkyl, C₁-C₃ alkoxy, C₁-C₂ haloalkyl, C₁-C₂ haloalkoxy, phenyl substituted with 0-3 R^{12c};

R^{12c}, at each occurrence, is independently selected from: H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, and C₁-C₄ haloalkoxy;

B is a 5 or 6 membered amino-heterocyclic ring, comprising one N atom, 3 to 5 carbon atoms, and optionally, an additional heteroatom —N(R^{LZ})—;

wherein the amino-heterocyclic ring is saturated or partially saturated; and

wherein R^{LZ} is either R¹⁰ or the substituent —L—Z;

R¹⁰ is H, C(═O)R¹⁷, C(═O)OR¹⁷, —(C₁-C₃ alkyl)—C(═O)OR¹⁷;

C₁-C₄ alkyl substituted with 0-1 R^{10a};

phenyl substituted with 0-4 R^{10b};

C₃-C₆ carbocycle substituted with 0-3 R^{10b}; or

5 to 6 membered heterocycle optionally substituted with 0-3 R^{10b};

R^{10a}, at each occurrence, is independently selected from: H, C₁-C₄ alkyl, OR¹⁴, Cl, F, Br, I, ═O, CN, NO₂, NR¹⁵R¹⁶, CF₃, or phenyl substituted with 0-4 R^{10b};

R^{10b}, at each occurrence, is independently selected from: H, OH, C₁-C₄ alkyl, C₁-C₃ alkoxy, Cl, F, Br, I, CN, NO₂, NR¹⁵R¹⁶, or CF₃;

R¹¹, at each occurrence, is independently selected from:

C₁-C₄ alkoxy, Cl, F, OH, NR¹⁸R¹⁹, C(═O)R¹⁷, C(═O)OR¹⁷, CF₃;

C₁-C₄ alkyl substituted with 0-1 R^{11a};

phenyl substituted with 0-3 R^{11b};

C₃-C₆ carbocycle substituted with 0-3 R^{11b}; or

5 to 6 membered heterocycle substituted with 0-3 R^{11b};

alternatively, two R¹¹ substituents on adjacent carbon atoms may be combined to form a cyclopropyl, cyclobutyl, cyclopentyl, cyclohexyl, or a benzo fused radical;

R^{11a}, at each occurrence, is independently selected from: H, C₁-C₄ alkyl, OR¹⁴, F, ═O, NR¹⁵R¹⁶, CF₃, or phenyl substituted with 0-3 R^{11b};

R^{11b}, at each occurrence, is independently selected from: H, OH, Cl, F, NR¹⁵R¹⁶, CF₃, C₁-C₄ alkyl, C₁-C₃ alkoxy, C₁-C₂ haloalkyl, and C₁-C₂ haloalkoxy;

t is 0, 1, or 2;

R¹³, at each occurrence, is independently selected from: H, OH, C₁-C₆ alkyl, C₁-C₄ alkoxy, Cl, F, Br, I, CN, NO₂, NR¹⁵R¹⁶, and CF₃;

R¹⁴ is H, phenyl, benzyl, C₁-C₄ alkyl, or C₂-C₄ alkoxyalkyl;

R¹⁵, at each occurrence, is independently selected from: H, C₁-C₄ alkyl, benzyl, phenethyl, —C(═O)-(C₁-C₄ alkyl), —S(═O)₂—(C₁-C₄ alkyl), and aryl;

R¹⁶, at each occurrence, is independently selected from: H, OH, C₁-C₄ alkyl, benzyl, phenethyl, —C(═O)—(C₁-C₄ alkyl) and —S(═O)₂—(C₁-C₄ alkyl);

alternatively, R¹⁵ and R¹⁶ on the same N atom may be combined to form a 5 to 6 membered heterocyclic fused radical, wherein said 5 to 6 membered heterocyclic is selected from pyrrolidonyl, piperidonyl, piperazinyl, and morpholinyl;

R¹⁷ is H, phenyl, benzyl, 4-fluorophenyl, 4-chlorophenyl, 4-methylphenyl, 4-trifluorophenyl, (4-fluorophenyl)methyl, (4-chlorophenyl)methyl, (4-methylphenyl)methyl, (4-trifluorophenyl)methyl, methyl, ethyl, propyl, butyl, methoxymethyl, methyoxyethyl, ethoxymethyl, or ethoxyethyl;

R¹⁸, at each occurrence, is independently selected from: H, methyl, ethyl, propyl, butyl, phenyl, benzyl, and phenethyl;

R¹⁹, at each occurrence, is independently selected from: H, methyl, and ethyl; and

alternatively, R¹⁸ and R¹⁹on the same N atom may be combined to form a 5 to 6 membered heterocyclic fused radical, wherein said 5 to 6 membered heterocyclic is selected from pyrrolidonyl, piperidonyl, piperazinyl, and morpholinyl.

[5] In another embodiment the present invention provides a compound of Formula (Ic):

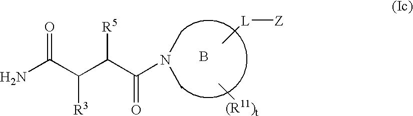

or a pharmaceutically acceptable salt or prodrug thereof, wherein:

R³ is C₁-C₄ alkyl, C₂-C₄ alkenyl, or C₂-C₄ alkynyl;

R⁵ is C₁-C₆ alkyl, C₂-C₆ alkenyl, or C₂-C₆ alkynyl;

L is a bond, —CH₂—, —CH₂CH₂—, —CH₂CH₂CH₂—, —CH₂CH═CH₂, —(CH₂)ₚ—O—(CH₂)_{q}—, or —(CH₂)ₚ—NR¹⁰—(CH₂)_{q}—;

p is 0, 1, 2, or 3;

q is 0, 1, or 2;

Z is C₃-C₁₀ carbocycle substituted with 0-2 R^{12b};

C₆-C₁₀ aryl substituted with 0-4 R^{12b}; and

5 to 10 membered heterocycle substituted with 0-5 R^{12b}, wherein the heterocycle contains 1, 2, 3 or 4 heteroatoms selected from N, O and S;

R^{12b}, at each occurrence, is independently selected from: H, OH, Cl, F, NR¹⁵R¹⁶, CF₃, acetyl, SCH₃, S(═O)CH₃, S(═O)₂CH₃, methyl, ethyl, propyl, butyl, methoxy, ethoxy, propoxy, C₁-C₂ haloalkyl, C₁-C₂ haloalkoxy, phenyl substituted with 0-3 R^{12c};

R^{12c}, at each occurrence, is independently selected from: H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, and C₁-C₄ haloalkoxy;

B is a 6 membered amino-heterocyclic ring, comprising one N atom, 4 or 5 carbon atoms, and optionally, an additional heteroatom —N(R^{LZ})—;

wherein the amino-heterocyclic ring is saturated or partially saturated; and

wherein R^{LZ} is either R¹⁰ or the substituent —L—Z;

R¹⁰ is H, C(═O)R¹⁷, C(═O)OR¹⁷, —(C₁-C₃ alkyl)—C(═O)OR¹⁷;

C₁-C₄ alkyl substituted with 0-1 R^{10a};

phenyl substituted with 0-4 R^{10b};

C₃-C₆ carbocycle substituted with 0-3 R^{10b}; or

5 to 6 membered heterocycle optionally substituted with 0-3 R^{10b};

R^{10a}, at each occurrence, is independently selected from: H, C₁-C₄ alkyl, OR¹⁴, Cl, F, Br, I, ═O, CN, NO₂, NR¹⁵R¹⁶, CF₃, or phenyl substituted with 0-4 R^{10b};

R^{10b}, at each occurrence, is independently selected from: H, OH, C₁-C₄ alkyl, C₁-C₃ alkoxy, Cl, F, Br, I, CN, NO₂, NR¹⁵R¹⁶, or CF₃;

R¹¹, at each occurrence, is independently selected from:

C₁-C₄ alkoxy, Cl, F, OH, NR¹⁸R¹⁹, C(═O)R¹⁷, C(═O)OR¹⁷, CF₃;

C₁-C₄ alkyl substituted with 0-1 R^{11a};

phenyl substituted with 0-3 R^{11b};

C₃-C₆ carbocycle substituted with 0-3 R^{11b}; or

5 to 6 membered heterocycle substituted with 0-3 R^{11b};

R^{11a}, at each occurrence, is independently selected from: H, C₁-C₄ alkyl, OR¹⁴, F, ═O, NR¹⁵R¹⁶, CF₃, or phenyl substituted with 0-3 R^{11b};

R^{11b}, at each occurrence, is independently selected from: H, OH, Cl, F, NR¹⁵R¹⁶, CF₃, C₁-C₄ alkyl, C₁-C₃ alkoxy, C₁-C₂ haloalkyl, and C₁-C₂ haloalkoxy;

t is 0, 1, or 2;

R¹³, at each occurrence, is independently selected from: H, OH, C₁-C₆ alkyl, C₁-C₄ alkoxy, Cl, F, Br, I, CN, NO₂, NR¹⁵R¹⁶, and CF₃;

R¹⁴ is H, phenyl, benzyl, methyl, ethyl, propyl, butyl;

R¹⁵, at each occurrence, is independently selected from: H, methyl, ethyl, propyl, butyl, and phenyl substituted with 0-3 substituents selected from OH, OCH₃, Cl, F, Br, I, CN, NO₂, NH₂, N(CH₃)H, N(CH₃)₂, CF₃, OCF₃, C(═O)CH₃, SCH₃, S(═O)CH₃, S(═O)₂CH₃, CH₃, CH₂CH₃, CO₂H, and CO₂CH₃;

R¹⁶, at each occurrence, is independently selected from: H, OH, C₁-C₄ alkyl, benzyl, phenethyl, —C(═O)—(C₁-C₄ alkyl) and —S(═O)₂—(C₁-C₄ alkyl);

alternatively, R¹⁵ and R¹⁶ on the same N atom may be combined to form a 5 to 6 membered heterocyclic fused radical, wherein said 5 to 6 membered heterocyclic is selected from pyrrolidonyl, piperidonyl, piperazinyl, and morpholinyl;

R¹⁷ is H, phenyl, benzyl, 4-fluorophenyl, 4-chlorophenyl, 4-methylphenyl, 4-trifluorophenyl, (4-fluorophenyl)methyl, (4-chlorophenyl)methyl, (4-methylphenyl) methyl, (4-trifluorophenyl) methyl, methyl, ethyl, propyl, butyl, methoxymethyl, methyoxyethyl, ethoxymethyl, or ethoxyethyl;

R¹⁸, at each occurrence, is independently selected from: H, methyl, ethyl, propyl, butyl, phenyl, benzyl, and phenethyl;

R¹⁹, at each occurrence, is independently selected from: H, methyl, ethyl, and

alternatively, R¹⁸ and R¹⁹ on the same N atom may be combined to form a 5 to 6 membered heterocyclic fused radical, wherein said 5 to 6 membered heterocyclic is selected from pyrrolidonyl, piperidonyl, piperazinyl, and morpholinyl.

[6] In another preferred embodiment the present invention provides a compound of Formula (Ib):

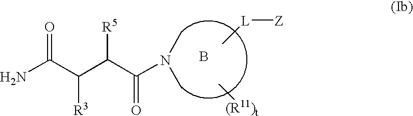

or a pharmaceutically acceptable salt or prodrug thereof, wherein:

R³ is —CH₃, —CH₂CH₃, —CH₂CH₂CH₃, —CH₂CH₂CH₂CH₃, —CH₂(CH₃)₂, —CH(CH₃)CH₂CH₃, —CH₂CH(CH₃)₂, —CH₂C(CH₃)₃, —CF₃, —CH₂CF₃, —CH₂CH₂CF₃, —CH₂CH₂CH₂CF₃;

—CH═CH₂, —CH₂CH═CH₂, —CH₂C(CH₃)═CH₂, —CH₂CH═C(CH₃)₂, —CH₂CH₂CH═CH₂, —CH₂CH₂C(CH₃)═CH₂, —CH₂CH₂CH═C(CH₃)₂, cis-CH₂CH═CH(CH₃), cis-CH₂CH₂CH═CH(CH₃), trans-CH₂CH═CH(CH₃), trans-CH₂CH₂CH═CH(CH₃);

—C≡CH, —CH₂C≡CH, —CH₂C≡C(CH₃); cyclopropyl-CH₂—, cyclobutyl-CH₂—, cyclopentyl-CH₂—, cyclohexyl-CH₂—, cyclopropyl-CH₂CH₂—, cyclobutyl-CH₂CH₂—, cyclopentyl-CH₂CH₂—, cyclohexyl-CH₂CH₂—;

phenyl-CH₂—, (2-F-phenyl)CH₂—, (3-F-phenyl)CH₂—, (4-F-phenyl)CH₂—, (2-Cl-phenyl)CH₂—, (3-Cl-phenyl)CH₂—, (4-Cl-phenyl)CH₂—, (2,3-diF-phenyl)CH₂—, (2,4-diF-phenyl)CH₂—, (2,5-diF-phenyl)CH₂—, (2,6-diF-phenyl)CH₂—, (3,4-diF-phenyl)CH₂—, (3,5-diF-phenyl)CH₂—, (2,3-diCl-phenyl)CH₂—, (2,4-diCl-phenyl)CH₂—, (2,5-diCl-phenyl)CH₂—, (2,6-diCl-phenyl)CH₂—, (3,4-diCl-phenyl)CH₂—, (3,5-diCl-phenyl)CH₂—, (3-F-4-Cl-phenyl)CH₂—, (3-F-5-Cl-phenyl)CH₂—, (3-Cl-4-F-phenyl)CH₂—, phenyl-CH₂CH₂—, (2-F-phenyl)CH₂CH₂—, (3-F-phenyl)CH₂CH₂—, (4-F-phenyl)CH₂CH₂—, (2-Cl-phenyl)CH₂CH₂—, (3-Cl-phenyl)CH₂CH₂—, (4-Cl-phenyl)CH₂CH₂—, (2,3-diF-phenyl)CH₂CH₂—, (2,4-diF-phenyl)CH₂CH₂—, (2,5-diF-phenyl)CH₂CH₂—, (2,6-diF-phenyl)CH₂CH₂—, (3,4-diF-phenyl)CH₂CH₂—, (3,5-diF-phenyl)CH₂CH₂—, (2,3-diCl-phenyl)CH₂CH₂—, (2,4-diCl-phenyl)CH₂CH₂—, (2,5-diCl-phenyl)CH₂CH₂—, (2,6-diCl-phenyl)CH₂CH₂—, (3,4-diCl-phenyl)CH₂CH₂—, (3,5-diCl-phenyl)CH₂CH₂—, (3-F-4-Cl-phenyl)CH₂CH₂—, or (3-F-5-Cl-phenyl)CH₂CH₂—;

R⁵ is —CH₃, —CH₂CH₃, —CH₂CH₂CH₃, —CH₂(CH₃)₂, —CH₂CH₂CH₂CH₃, —CH(CH₃)CH₂CH₃, —CH₂CH(CH₃)₂, —CH₂C(CH₃)₃, —CH₂CH₂CH₂CH₂CH₃, —CH(CH₃)CH₂CH₂CH₃, —CH₂CH(CH₃)CH₂CH₃, —CH₂CH₂CH(CH₃)₂, —CH(CH₂CH₃)₂, —CF₃, —CH₂CF₃, —CH₂CH₂CF₃, —CH₂CH₂CH₂CF₃, —CH₂CH₂CH₂CH₂CF₃, —CH═CH₂, —CH₂CH═CH₂, —CH═CHCH₃, —CH₂C(CH₃)═CH₂, cis-CH₂CH═CH(CH₃), trans-CH₂CH═CH(CH₃), trans-CH₂CH═CH(C₆H₅), —CH₂CH═C(CH₃)₂, cis-CH₂CH═CHCH₂CH₃, trans-CH₂CH═CHCH₂CH₃, cis-CH₂CH₂CH═CH(CH₃), trans-CH₂CH₂CH═CH(CH₃), trans-CH₂CH═CHCH₂(C₆H₅), —C≡CH, —CH₂C≡CH, —CH₂C≡C(CH₃), —CH₂C≡C (C₆H₅), —CH₂CH₂C≡CH, —CH₂CH₂C≡C(CH₃), —CH₂CH₂C≡C(C₆H₅), —CH₂CH₂CH₂C≡CH, —CH₂CH₂CH₂C≡C(CH₃), —CH₂CH₂CH₂C≡C(C₆H₅), cyclopropyl-CH₂—, cyclobutyl-CH₂—, cyclopentyl-CH₂—, cyclohexyl-CH₂—, (2-CH₃-cyclopropyl)CH₂—, (3-CH₃-cyclobutyl)CH₂—, cyclopropyl-CH₂CH₂—, cyclobutyl-CH₂CH₂—, cyclopentyl-CH₂CH₂—, cyclohexyl-CH₂CH₂—, (2-CH₃-cyclopropyl)CH₂CH₂—, (3-CH₃-cyclobutyl)CH₂CH₂—, phenyl-CH₂—, (2-F-phenyl)CH₂—, (3-F-phenyl)CH₂—, (4-F-phenyl)CH₂—, furanyl-CH₂—, thienyl-CH₂—, pyridyl-CH₂—, 1-imidazolyl-CH₂—, oxazolyl-CH₂—, isoxazolyl-CH₂—, phenyl-CH₂CH₂—, (2-F-phenyl)CH₂CH₂—, (3-F-phenyl)CH₂CH₂—, (4-F-phenyl)CH₂CH₂—, furanyl-CH₂CH₂—, thienyl-CH₂CH₂—, pyridyl-CH₂CH₂—, 1-imidazolyl-CH₂CH₂—, oxazolyl-CH₂CH₂—, or isoxazolyl-CH₂CH₂—;

L is a bond, —CH₂—, —CH₂CH₂—, —CH₂CH₂CH₂—, —CH₂CH═CH₂, O, —CH₂O—, —(CH₂)₂—O—, —(CH₂)₃—O—, —(CH₂)—O—(CH₂)₂—, —(CH₂)₂—O—(CH₂)—, —(CH₂)₂—O—(CH₂)₂—, NH, NMe, —CH₂NH—, —(CH₂)₂—NH—, —(CH₂)₃—NH—, —(CH₂)—NH—(CH₂)₂—, —(CH₂)₂—NH—(CH₂)—, —(CH₂)₂—NH—(CH₂)₂—, and —N(benzoyl)-;

Z is cyclopropyl, cyclobutyl, cyclopentyl, cyclohexyl, phenyl 2-F-phenyl, 3-F-phenyl, 4-F-phenyl, 2-Cl-phenyl, 3-Cl-phenyl, 4-Cl-phenyl, 2,3-diF-phenyl, 2,4-diF-phenyl, 2,5-diF-phenyl, 2,6-diF-phenyl, 3,4-diF-phenyl, 3,5-diF-phenyl, 2,3-diCl-phenyl, 2,4-diCl-phenyl, 2,5-diCl-phenyl, 2,6-diCl-phenyl, 3,4-diCl-phenyl, 3,5-diCl-phenyl, 2,3-diMe-phenyl, 2,4-diMe-phenyl, 2,5-diMe-phenyl, 2,6-diMe-phenyl, 3,4-diMe-phenyl, 3,5-diMe-phenyl, 2,3-diMeO-phenyl, 2,4-diMeO-phenyl, 2,5-diMeO-phenyl, 2,6-diMeO-phenyl, 3,4-diMeO-phenyl, 3,5-diMeO-phenyl, 3-F-4-Cl-phenyl, 3-F-5-Cl-phenyl, 3-Cl-4-F-phenyl, 2-MeO-phenyl, 3-MeO-phenyl, 4-MeO-phenyl, 2-EtO-phenyl, 3-EtO-phenyl, 4-EtO-phenyl, 2-Me-phenyl, 3-Me-phenyl, 4-Me-phenyl, 2-Et-phenyl, 3-Et-phenyl, 4-Et-phenyl, 2-CF₃-phenyl, 3-CF₃-phenyl, 4-CF₃-phenyl, 2-NO₂-phenyl, 3-NO₂-phenyl, 4-NO₂-phenyl, 2-CN-phenyl, 3-CN-phenyl, 4-CN-phenyl, 2-MeS-phenyl, 3-MeS-phenyl, 4-MeS-phenyl, 2-CF₃O-phenyl, 3-CF₃O-phenyl, 4-CF₃O-phenyl, 2-Me-5-Cl-phenyl, 3-CF₃-4-Cl-phenyl, 3-CF₃-5-F-phenyl, 3-MeO-4-Me-phenyl, furanyl, thienyl, pyrid-2-yl, pyrid-3-yl, pyrid-4-yl, pyrimidyl, pyrazinyl, 2-Me-pyridyl, 3-Me-pyridyl, 3-CF₃-pyrid-2-yl, 5-CF₃-pyrid-2-yl, 4-Me-pyridyl, pyrrolidinyl, 1-imidazolyl, oxazolyl, isoxazolyl, 1-benzimidazolyl, 2-keto-1-benzimidazolyl, 4-benzo[1,3]dioxol-5-yl, morpholino, N-piperidyl, 4-piperidyl, naphthyl, 4(phenyl)phenyl-, 4(4-CF₃-phenyl)phenyl-, 3,5-bis-CF₃-phenyl-, 4-iPr-phenyl-, N-piperidino-CH₂—, 1-Me-pyrrolidin-2-yl, and 1-pyrrolidinyl;

B is a 5 or 6 membered amino-heterocyclic ring, comprising one N atom, 3 to 5 carbon atoms, and optionally, an additional heteroatom —N(R^{LZ})—;

wherein the amino-heterocyclic ring is saturated or partially saturated; and

wherein R^{LZ} is either R¹⁰ or the substituent —L—Z;

R¹⁰ is H, methyl, ethyl, phenyl, benzyl, phenethyl, 4-F-phenyl, (4-F-phenyl)CH₂—, (4-F-phenyl)CH₂CH₂—, 4-Cl-phenyl, (4-Cl-phenyl)CH₂—, (4-Cl-phenyl)CH₂CH₂—, 4-CH₃-phenyl, (4-CH₃-phenyl)CH₂—, (4-CH₃-phenyl)CH₂CH₂—, 4-CF₃-phenyl, (4-CF₃-phenyl)CH₂—, (4-CF₃-phenyl)CH₂CH₂—, —CH₂C(═O)Et, —C(═O)Me, or 4-Cl-benzhydryl;

R¹¹, at each occurrence, is independently selected from: H, OH, methyl, ethyl, —CN, —C(═O)Me, —C(═O)OEt, —C(═O)Et, —CH₂OH, —C(═O)NH₂, —C(═O)OH, —C(═O)N(Et)₂, phenyl, benzyl, phenethyl, 4-F-phenyl, (4-F-phenyl)CH₂—, (4-F-phenyl)CH₂CH₂—, 4-Cl-phenyl, (4-Cl-phenyl)CH₂—, (4-Cl-phenyl)CH₂CH₂—, 4-CH₃-phenyl, (4-CH₃-phenyl)CH₂—, (4-CH₃-phenyl)CH₂CH₂—, 4-CF₃-phenyl, (4-CF₃-phenyl)CH₂—, (4-CF₃-phenyl)CH₂CH₂—, and —N(Me)₂—,; and

t is 0, 1, or 2;

alternatively, two R¹¹ substituents on the same or adjacent carbon atoms may be combined to form a cyclopropyl, cyclobutyl, cyclopentyl, cyclohexyl, or a benzo fused radical.

[7] In another preferred embodiment the present invention provides a compound of Formula (Ib):

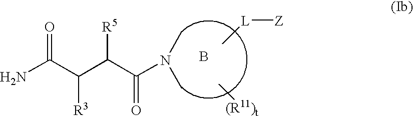

or a pharmaceutically acceptable salt or prodrug thereof, wherein:

R³ is —CH₂CH₂CH₃, —CH₂CH₂CH₂CH₃, —CH₂(CH₃)₂, —CH₂CH(CH₃)₂, —CH₂CH═CH₂, —CH₂CH₂CH═CH₂, —CH₂CH₂CH═C(CH₃)₂, cis-CH₂CH═CH(CH₃), cis-CH₂CH₂CH═CH(CH₃), trans-CH₂CH═CH(CH₃), trans-CH₂CH₂CH═CH(CH₃); cyclopropyl-CH₂—, cyclobutyl-CH₂—, cyclopentyl-CH₂—, cyclohexyl-CH₂—, cyclopropyl-CH₂CH₂—, cyclobutyl-CH₂CH₂—, cyclopentyl-CH₂CH₂—, or cyclohexyl-CH₂CH₂—;

R⁵ is —CH₂ (CH₃)₂, —CH₂CH₂CH₂CH₃, —CH(CH₃)CH₂CH₃, —CH₂CH(CH₃)₂, —CH₂C (CH₃)₃, —CH₂CH₂CH₂CH₂CH₃, —CH(CH₃)CH₂CH₂CH₃, —CH₂CH(CH₃)CH₂CH₃, —CH₂CH₂CH(CH₃)₂, —CH (CH₂CH₃)₂, —CH₂CH═CH₂, —CH₂C(CH₃)═CH₂, cis-CH₂CH═CH(CH₃), trans-CH₂CH═CH(CH₃), —CH₂CH═C(CH₃)₂, cyclopropyl-CH₂—, cyclobutyl-CH₂—, cyclopentyl-CH₂—, cyclohexyl-CH₂—, (2-CH₃-cyclopropyl)CH₂—, or (3-CH₃-cyclobutyl)CH₂—,

L is a bond, —CH₂—, —CH₂CH₂—, —CH₂CH₂CH₂—, —CH₂CH═CH₂, O, —CH₂O—, —(CH₂)₂—O—, —(CH₂)₃—O—, —(CH₂)—O—(CH₂)₂—, —(CH₂)₂—O—(CH₂)—, —(CH₂)₂—O—(CH₂)₂—, NH, NMe, —CH₂NH—, —(CH₂)₂—NH—, —(CH₂)₃—NH—, —(CH₂)—NH—(CH₂)₂—, —(CH₂)₂—NH—(CH₂)—, —(CH₂)₂—NH—(CH₂)₂—, and —N(benzoyl)-;

Z is cyclopropyl, cyclobutyl, cyclopentyl, cyclohexyl, phenyl 2-F-phenyl, 3-F-phenyl, 4-F-phenyl, 2-Cl-phenyl, 3-Cl-phenyl, 4-Cl-phenyl, 2,3-diF-phenyl, 2,4-diF-phenyl, 2,5-diF-phenyl, 2,6-diF-phenyl, 3,4-diF-phenyl, 3,5-diF-phenyl, 2,3-diCl-phenyl, 2,4-diCl-phenyl, 2,5-diCl-phenyl, 2, 6-diCl-phenyl, 3,4-diCl-phenyl, 3,5-diCl-phenyl, 2,3-diMe-phenyl, 2,4-diMe-phenyl, 2,5-diMe-phenyl, 2,6-dime-phenyl, 3,4-diMe-phenyl, 3,5-diMe-phenyl, 2,3-diMeO-phenyl, 2,4-diMeO-phenyl, 2,5-diMeO-phenyl, 2,6-diMeO-phenyl, 3,4-diMeO-phenyl, 3,5-diMeO-phenyl, 3-F-4-Cl-phenyl, 3-F-5-Cl-phenyl, 3-Cl-4-F-phenyl, 2-MeO-phenyl, 3-MeO-phenyl, 4-MeO-phenyl, 2-EtO-phenyl, 3-EtO-phenyl, 4-EtO-phenyl, 2-Me-phenyl, 3-Me-phenyl, 4-Me-phenyl, 2-Et-phenyl, 3-Et-phenyl, 4-Et-phenyl, 2-CF₃-phenyl, 3-CF₃-phenyl, 4-CF₃-phenyl, 2-NO₂-phenyl, 3-NO₂-phenyl, 4-NO₂-phenyl, 2-CN-phenyl, 3-CN-phenyl, 4-CN-phenyl, 2-MeS-phenyl, 3-MeS-phenyl, 4-MeS-phenyl, 2-CF₃O-phenyl, 3-CF₃O-phenyl, 4-CF₃O-phenyl, 2-Me-5-Cl-phenyl, 3-CF₃-4-Cl-phenyl, 3-CF₃-5-F-phenyl, 3-MeO-4-Me-phenyl, furanyl, thienyl, pyrid-2-yl, pyrid-3-yl, pyrid-4-yl, pyrimidyl, pyrazinyl, 2-Me-pyridyl, 3-Me-pyridyl, 3-CF₃-pyrid-2-yl, 5-CF₃-pyrid-2-yl, 4-Me-pyridyl, pyrrolidinyl, 1-imidazolyl, oxazolyl, isoxazolyl, 1-benzimidazolyl, 2-keto-1-benzimidazolyl, 4-benzo[1,3]dioxol-5-yl, morpholino, N-piperidyl, 4-piperidyl, naphthyl, 4(phenyl)phenyl-, 4(4-CF₃-phenyl)phenyl-, 3,5-bis-CF₃-phenyl-, 4-iPr-phenyl-, N-piperidino-CH₂—, 1-Me-pyrrolidin-2-yl, and 1-pyrrolidinyl;

B is a 5 or 6 membered amino-heterocyclic ring, comprising one N atom, 3 to 5 carbon atoms, and optionally, an additional heteroatom —N(R^{LZ})—;

wherein the amino-heterocyclic ring is saturated or partially saturated; and

wherein R^{LZ} is the substituent —L—Z;

R¹¹, at each occurrence, is independently selected from: H, OH, methyl, ethyl, —CN, —C(═O)Me, —C(═O)OEt, —C(═O)Et, —CH₂OH, —C(═O)NH₂, —C(═O)OH, —C(═O)N(Et)₂, and —N(Me)₂—;

t is 0 or 1.

In another preferred embodiment the present invention provides a compound of the present invention wherein B is

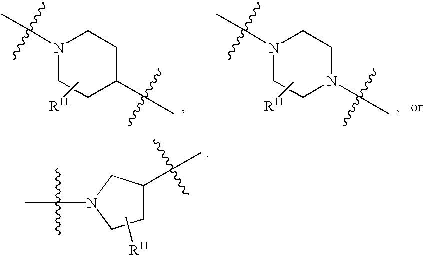

In another preferred embodiment the present invention provides a compound of the present invention wherein B is

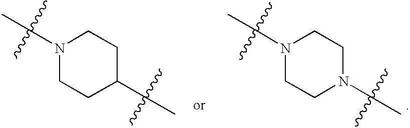

In another preferred embodiment the present invention provides a compound selected from one of the Examples in Table 5a, Table 5b, Table 5c, Table 5d, Table 5e, Table 5f or Table 5g.

In another even further more preferred embodiment the present invention provides for a compound selected from:

5-Methyl-2-propyl-3-[4-(3-trifluoromethyl-phenyl)-piperazine-1-carbonyl]-hexanoic acid amide;

3-[4-(5-Chloro-2-methyl-phenyl)-piperazine-1-carbonyl]-5-methyl-2-propyl-hexanoic acid amide;

3-[3-Hydroxy-4-(3-trifluoromethyl-phenyl)-piperidine-1-carbonyl]-5-methyl-2-propyl-hexanoic acid amide;

3-[4-(3,4-Dichloro-phenyl)-piperazine-1-carbonyl]-5-methyl-2-propyl-hexanoic acid amide;

3-[4-(4-Chloro-3-trifluoromethyl-phenyl)-piperazine-1-carbonyl]-5-methyl-2-propyl-hexanoic acid amide;

3-[4-(4-Chloro-3-trifluoromethyl-phenyl)-4-hydroxy-piperidine-1-carbonyl]-5-methyl-2-propyl-hexanoic acid amide;

5-Methyl-3-(4-phenyl-piperidine-1-carbonyl)-2-propyl-hexanoic acid amide;

3-(3-Benzyl-pyrrolidine-1-carbonyl)-5-methyl-2-propyl-hexanoic acid amide;

5-Methyl-3-(4-phenyl-piperidine-1-carbonyl)-2-propyl-hexanoic acid amide; and

3-(3-Benzyl-pyrrolidine-1-carbonyl)-5-methyl-2-propyl-hexanoic acid amide.

In another preferred embodiment

R³ is R⁴,

R^{3a} is H, methyl, ethyl, propyl, or butyl;

R⁴ is C₁-C₆ alkyl, C₂-C₆ alkenyl, C₂-C₆ alkynyl

R⁵ is C₁-C₆ alkyl, C₂-C₆ alkenyl, C₂-C₆ alkynyl

R^{5a}is H, methyl, ethyl, propyl, or butyl; and

the total number of carbon atoms in R³, R^{3a}, R⁵ and R^{5a} equals seven or more.

In another preferred embodiment

R³ is C₃-C₄ alkyl or C₃-C₄ alkenyl,

R^{3a} is H;

R⁵ is C₃-C₅ alkyl or C₃-C₅ alkenyl, and

R^{5a} is H.

In another preferred embodiment

R³ is R⁴;

R^{3a} is H;

R⁴ is C₁-C₄ alkyl substituted with 1-2 R^{4a},

R^{4a}, at each occurrence, is independently selected from C₃-C₆ cycloalkyl substituted with 0-3 R^{4b}, phenyl substituted with 0-3 R^{4b}, or 5 to 6 membered heterocycle substituted with 0-3 R^{4b};

R^{4b}, at each occurrence, is independently selected from H, OH, Cl, F, NR¹⁵R¹⁶, CF₃, acetyl, SCH₃, S(═O)CH₃, S(═O)₂CH₃, methyl, ethyl, propyl, butyl, methoxy, ethoxy, propoxy, C₁-C₂ haloalkyl, and C₁-C₂ haloalkoxy;

R⁵ is C₂-C₄ alkyl substituted with 0-3 R^{5b};

C₂-C₄ alkenyl substituted with 0-2 R^{5b}; or

C₂-C₄ alkynyl substituted with 0-2 R^{5b};

R^{5b}, at each occurrence, is independently selected from:

H, methyl, ethyl, propyl, butyl, CF₃, OR¹⁴, ═O;

C₃-C₆ cycloalkyl substituted with 0-2 R^{5c};

phenyl substituted with 0-3 R^{5c}; or

5 to 6 membered heterocycle substituted with 0-2 R^{5c}; and

R^{5c}, at each occurrence, is independently selected from H, OH, Cl, F, NR¹⁵R¹⁶, CF₃, acetyl, SCH₃, S(═O)CH₃, S(═O)₂CH₃, methyl, ethyl, propyl, butyl, methoxy, ethoxy, propoxy, C₁-C₂ haloalkyl, and C₁-C₂ haloalkoxy.

In another preferred embodiment

R³ is R⁴;

R^{3a} is H;

R⁴ is C₂-C₄ alkyl substituted with 0-2 R^{4a},

C₂-C₄ alkenyl substituted with 0-2 R^{4a},

C₂-C₄ alkynyl substituted with 0-2 R^{4a},

R^{4a}, at each occurrence, is independently selected from is H, F, CF₃,

C₃-C₆ cycloalkyl substituted with 0-3 R^{4b},

phenyl substituted with 0-3 R^{4b}, or

5 to 6 membered heterocycle substituted with 0-3 R^{4b};

R^{4b}, at each occurrence, is independently selected from H, OH, Cl, F, NR¹⁵R¹⁶, CF₃, acetyl, SCH₃, S(═O)CH₃, S(═O)₂CH₃, methyl, ethyl, propyl, butyl, methoxy, ethoxy, propoxy, C₁-C₂ haloalkyl, and C₁-C₂ haloalkoxy;

R⁵ is C₁-C₄ alkyl substituted with 1-2 R^{5b};

R^{5b}, at each occurrence, is independently selected from:

C₃-C₆ cycloalkyl substituted with 0-2 R^{5c};

phenyl substituted with 0-3 R^{5c}; or

5 to 6 membered heterocycle substituted with 0-2 R^{5c}; and

R^{5c}, at each occurrence, is independently selected from H, OH, Cl, F, NR¹⁵R¹⁶, CF₃, acetyl, SCH₃, S(═O)CH₃, S(═O)₂CH₃, methyl, ethyl, propyl, butyl, methoxy, ethoxy, propoxy, C₁-C₂ haloalkyl, and C₁-C₂ haloalkoxy.

Also included in the present invention in a preferred embodiment are compounds as set forth above wherein the total number of carbon atoms in R³, R^{3a}, R⁵, and R^{5a}, equals four or more.

Also included in the present invention in a preferred embodiment are compounds as set forth above wherein the total number of carbon atoms in R³, R^{3a}, R⁵, and R^{5a}, equals seven or more.

Also included in the present invention in a preferred embodiment are compounds as set forth above wherein R^{3a} and R^{5a} are hydrogen, and R³ and R⁵ are not hydrogen.

It is understood that any and all embodiments of the present invention may be taken in conjunction with any other embodiment to descibe additional even more preferred embodiments of the present invention.

In a second embodiment, the present invention provides a pharmaceutical composition comprising a compound of Formula (I) and a pharmaceutically acceptable carrier.

In a third embodiment, the present invention provides a method for the treatment of neurological disorders associated with β-amyloid production comprising administering to a host in need of such treatment a therapeutically effective amount of a compound of Formula (I).

In a preferred embodiment the neurological disorder associated with β-amyloid production is Alzheimer's Disease.

In a fourth embodiment, the present invention provides a method for inhibiting γ-secretase activity for the treatment of a physiological disorder associated with inhibiting γ-secretase activity comprising administering to a host in need of such inhibition a therapeutically effective amount of a compound of Formula (I) that inhibits γ-secretase activity.

In a preferred embodiment the physiological disorder associated with inhibiting γ-secretase activity is Alzheimer's Disease.

In a fifth embodiment, the present invention provides a compound of Formula (I) for use in therapy.

In a preferred embodiment the present invention provides a compound of Formula (I) for use in therapy of Alzheimer's Disease.

In a sixth embodiment, the present invention provides for the use of a compound of Formula (I) for the manufacture of a medicament for the treatment of Alzheimer's Disease.

### Definitions

As used herein, the term “Aβ” denotes the protein designated Aβ, β-amyloid peptide, and sometimes β/A4, in the art. Aβ is an approximately 4.2 kilodalton (kD) protein of about 39 to 43 amino acids found in amyloid plaques, the walls of meningeal and parenchymal arterioles, small arteries, capillaries, and sometimes, venules. The isolation and sequence data for the first 28 amino acids are described in U.S. Pat. No. 4,666,829. The 43 amino acid sequence is:

1
Asp Ala Glu Phe Arg His Asp Ser Gly Tyr
11
Glu Val His His Gln Lys Leu Val Phe Phe
21
Ala Glu Asp Val Gly Ser Asn Lys Gly Ala
31
Ile Ile Gly Leu Met Val Gly Gly Val Val
41
Ile Ala Thr.

However, a skilled artisan knows that fragments generated by enzymatic degradation can result in loss of amino acids 1-10 and/or amino acids 39-43. Thus, an amino acid sequence 1-43 represents the maximum sequence of amino acids for Aβ peptide.

The term “APP”, as used herein, refers to the protein known in the art as β amyloid precursor protein. This protein is the precursor for Aβ and through the activity of “secretase” enzymes, as used herein, it is processed into Aβ. Differing secretase enzymes, known in the art, have been designated β secretase, generating the N-terminus of Aβ, a secretase cleaving around the 16/17 peptide bond in Aβ, and “γ secretases”, as used herein, generating C-terminal Aβ fragments ending at position 38, 39, 40, 41, 42, and 43 or generating C-terminal extended precursors which are subsequently truncated to the above polypeptides.

The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced.

When any variable (e.g. , R^{4b}, R^{5b}, R^{11b}, R^{12b}, etc.) occurs more than one time in any constituent or formula for a compound, its definition at each occurrence is independent of its definition at every other occurrence. Thus, for example, if a group is shown to be substituted with 0-2 R^{5b}, then said group may optionally be substituted with up to two R^{5b} groups and R^{5b} at each occurrence is selected independently from the definition of R^{5b}. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein, “alkyl” or “alkylene” is intended to include both branched and straight-chain saturated aliphatic hydrocarbon groups having the specified number of carbon atoms; for example, “C₁-C₆ alkyl” denotes alkyl having 1, 2, 3, 4, 5 and 6 carbon atoms. Examples of alkyl include, but are not limited to, methyl, ethyl, n-propyl, i-propyl, n-butyl, i-butyl, sec-butyl, t-butyl, pentyl, and hexyl. Preferred “alkyl” group, unless otherwise specified, is “C₁-C₄ alkyl”, more preferred is methyl, ethyl, propyl, and butyl.

As used herein, “alkenyl” or “alkenylene” is intended to include hydrocarbon chains of either a straight or branched configuration and one or more unsaturated carbon-carbon bonds which may occur in any stable point along the chain. Examples of “C₂-C₆ alkenyl” include, but are not limited to, ethenyl, 1-propenyl, 2-propenyl, 1-butenyl, 2-butenyl, 3-butenyl, 3-methyl-2-butenyl, 2-pentenyl, 3-pentenyl, hexenyl, and the like.

As used herein, “alkynyl” or “alkynylene” is intended to include hydrocarbon chains of either a straight or branched configuration and one or more carbon-carbon triple bonds which may occur in any stable point along the chain. Examples of “C₂-C₆ alkynyl” include, but are not limited to, ethynyl, 1-propynyl, 2-propynyl, 1-butynyl, 2-butynyl, 3-butynyl, and the like.

“Alkoxy” or “alkyloxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy. Similarly, “alkylthio” or “thioalkoxy” represents an alkyl group as defined above with the indicated number of carbon atoms attached through a sulphur bridge.

“Halo” or “halogen” as used herein refers to fluoro, chloro, bromo, and iodo. Unless otherwise specified, preferred halo is fluoro and chloro. “Counterion” is used to represent a small, negatively charged species such as chloride, bromide, hydroxide, acetate, sulfate, and the like.

“Haloalkyl” is intended to include both branched and straight-chain saturated aliphatic hydrocarbon groups having the specified number of carbon atoms, substituted with 1 or more halogen (for example —CᵥF_{w} where v=1 to 3 and w=1 to (2v+1)). Examples of haloalkyl include, but are not limited to, trifluoromethyl, trichloromethyl, pentafluoroethyl, pentachloroethyl, 2,2,2-trifluoroethyl, 2,2-difluoroethyl, heptafluoropropyl, and heptachloropropyl. “Haloalkoxy” is intended to mean a haloalkyl group as defined above with the indicated number of carbon atoms attached through an oxygen bridge; for example trifluoromethoxy, pentafluoroethoxy, 2,2,2-trifluoroethoxy, and the like. “Halothioalkoxy” is intended to mean a haloalkyl group as defined above with the indicated number of carbon atoms attached through a sulphur bridge.

“Cycloalkyl” is intended to include saturated ring groups, having the specified number of carbon atoms. For example, “C₃-C₆ cycloalkyl” denotes such as cyclopropyl, cyclobutyl, cyclopentyl, or cyclohexyl.

As used herein, “carbocycle” is intended to mean any stable 3, 4, 5, 6 and 7-membered monocyclic or bicyclic or 7, 8, 9, 10, 11, 12 and 13-membered bicyclic or tricyclic, any of which may be saturated, partially unsaturated, or aromatic. Examples of such carbocycles include, but are not limited to, cyclopropyl, cyclobutyl, cyclopentyl, cyclohexyl, cycloheptyl, adamantyl, cyclooctyl, [3.3.0]bicyclooctane, [4.3.0]bicyclononane, [4.4.0]bicyclodecane (decalin), [2.2.2]bicyclooctane, fluorenyl, phenyl, naphthyl, indanyl, adamantyl, or tetrahydronaphthyl (tetralin). Preferred “carbocycle” are cyclopropyl, cyclobutyl, cyclopentyl, and cyclohexyl.

As used herein, the term “heterocycle” or “heterocyclic ring” is intended to mean a stable 5, 6, and 7-membered monocyclic or bicyclic or 7, 8, 9, 10, 11, 12, 13 and 14-membered bicyclic heterocyclic ring which is saturated partially unsaturated or unsaturated (aromatic), and which consists of carbon atoms and 1, 2, 3 or 4 heteroatoms, preferably 1, 2, or 3 heteroatoms, independently selected from the group consisting of N, O and S and including any bicyclic group in which any of the above-defined heterocyclic rings is fused to a benzene ring. The nitrogen and sulfur heteroatoms may optionally be oxidized. The heterocyclic ring may be attached to its pendant group at any heteroatom or carbon atom which results in a stable structure. The heterocyclic rings described herein may be substituted on carbon or on a nitrogen atom if the resulting compound is stable. If specifically noted, a nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1.

Examples of heterocycles include, but are not limited to, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, but are not limited to, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, tetrazolyl; more preferred 5 to 6 membered heterocycles include, but are not limited to, pyridinyl, pyrimidinyl, triazinyl, furanyl, thienyl, thiazolyl, piperazinyl, piperidinyl, pyrazolyl, imidazolyl, and tetrazolyl. Also included are fused ring and spiro compounds containing, for example, the above heterocycles.

As used herein, the term “aryl”, “C₆-C₁₀ aryl” or aromatic residue, is intended to mean an aromatic moiety containing the specified number of carbon atoms; for example phenyl, pyridinyl or naphthyl; preferably phenyl or naphthyl. Unless otherwise specified, “aryl” may be unsubstituted or substituted with 0 to 3 groups selected from H, OH, OCH₃, Cl, F, Br, I, CN, NO₂, NH₂, N(CH₃)H, N(CH₃)₂, CF₃, OCF₃, C(═O)CH₃, SCH₃, S(═O)CH₃, S(═O)₂CH₃, CH₃, CH₂CH₃, CO₂H, and COCH₃.

The phrase “amino-heterocyclic ring”, as used herein, is intended to denote a heterocyclic ring of Formula (I″)

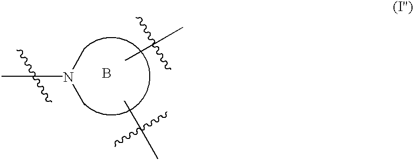

comprising at least one nitrogen atom, carbon atoms and optionally a second additional heteroatom selected from oxygen, nitrogen and sulfur; wherein the total number of members of “amino-heterocycle ring” B does not exceed 8. When “amino-heterocycle ring” B comprises one nitrogen atom, then amino-heterocyclic ring B also contains 3, 4, 5, 6 or 7 carbons. Alternatively, when “amino-heterocycle ring” B comprises one nitrogen atom and a second additional heteroatom, then amino-heterocyclic ring B contains 3, 4, 5, or 6 carbons. It is preferred that the total number of atoms of amino-heterocyclic ring B is 5, 6, or 7; it is more preferred that the total number of atoms of amino-heterocyclic ring B is five or six.

It is further understood that amino-heterocyclic ring B may be saturated or partially unsaturated (i.e. two adjacent atoms in the ring form a double bond) wherein the backbone of amino-heterocyclic ring B may contain one, two or three double bonds, but not fully unsaturated. Examples of amino-heterocyclic ring B include, but are not limited to piperidine, piperazine, and pyrrolidine.

It is further understood that amino-heterocyclic ring B may contain a second additional heteroatom selected from oxygen, nitrogen and sulfur; for example —O—, —S—, —S(═O)—, —S(═O)₂—, —N═, and —N(R^{LZ})—. When the second additional heteroatom is selected from oxygen and sulfur; then substituent —L—Z of Formula (I) is attached to amino-heterocyclic ring B through a ring carbon. When the second additional heteroatom is selected from nitrogen, then substituent —L—Z of Formula (I) is attached to amino-heterocyclic ring B through the second nitrogen or through a ring carbon. When substituent —L—Z of Formula (I) is attached to amino-heterocyclic ring B through the second nitrogen the second nitrogen is designated as —N(R^{LZ})—. Alternatively, when substituent —L—Z of Formula (I) is attached to amino-heterocyclic ring B through a ring carbon then the second nitrogen is designated as —N(R¹⁰)— or —N═.

It is further understood that amino-heterocyclic ring B may be substituted with 0, 1, 2, or 3 R¹¹ groups. Such R¹¹ groups are substituted on amino-heterocyclic ring B through the ring carbon atoms. It is understood when amino-heterocyclic ring B is substituted with 2 or 3 R¹¹ groups then two such R¹¹ groups may be substituted in the same or adjacent carbon.

The compounds herein described may have asymmetric centers. One enantiomer of a compound of Formula (I) may display superior chemical activity over the opposite enantiomer. When required, separation of the racemic material can be achieved by methods known in the art. For example, the carbon atoms to which R³ and R⁵ are attached may describe chiral carbons which may display superior chemical activity over the opposite enantiomer. For example, where R³ and R⁵ are not H, then the configuration of the two centers may be described as (2R,3R), (2R,3S), (2S,3R), or (2S,3S). All configurations are considered part of the invention; however, the (2R,3S) and the (2S,3R) are preferred and the (2R,3S) is more preferred.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

“Prodrugs” are intended to include any covalently bonded carriers which release the active parent drug according to Formula (I) in vivo when such prodrug is administered to a mammalian subject. Prodrugs of a compound of Formula (I) are prepared by modifying functional groups present in the compound in such a way that the modifications are cleaved, either in routine manipulation or in vivo, to the parent compound. Prodrugs include compounds of Formula (I) wherein a hydroxy, amino, or sulfhydryl group is bonded to any group that, when the prodrug or compound of Formula (I) is administered to a mammalian subject, cleaves to form a free hydroxyl, free amino, or free sulfhydryl group, respectively. Examples of prodrugs include, but are not limited to, acetate, formate and benzoate derivatives of alcohol and amine functional groups in the compounds of Formula (I), and the like.

“Stable compound” and “stable structure” are meant to indicate a compound that is sufficiently robust to survive isolation to a useful degree of purity from a reaction mixture, and formulation into an efficacious therapeutic agent.

As used herein the term “effective amount” means an amount of a compound/composition according to the present invention effective in producing the desired therapeutic effect.

As used herein the term treating or “treatment” refers to: (i) preventing a disease, disorder or condition from occurring in an animal which may be predisposed to the disease, disorder and/or condition but has not yet been diagnosed as having it; (ii) inhibiting the disease, disorder or condition, i.e., arresting its development; and (iii) relieving the disease, disorder or condition, i.e., causing regression of the disease, disorder and/or condition.

As used herein the term “patient” or “host” includes both human and other mammals.

### Synthesis

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Preferred methods include, but are not limited to, those described below. All references cited herein are hereby incorporated in their entirety herein by reference.

The novel compounds of this invention may be prepared using the reactions and techniques described in this section. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents which are compatible with the reaction conditions will be readily apparent to one skilled in the art and alternate methods must then be used.

Patent publication WO 00/07995 and U.S. patent application Ser. No. 09/505,788 both describe synthesis of succinate derivatives. The synthetic disclosure of each of these applications is hereby incorporated by reference.

Disubstituted succinate derivatives can be prepared by a number of known procedures. The procedure of Evans (D. A. Evans et al, Org. Synth. 86, p83 (1990)) is outlined in Scheme 1 where acylation of an oxazolidinone with an acylating agent such as an acid chloride provides structures 1. Alkylation to form 2 followed by cleavage of the chiral auxiliary and subsequent alkylation of the dianion of the carboxylic acid 3 provides a variety of disubstituted succinates which can be separated and incorporated into structures of Formula (I) by those skilled in the art. Additional examples are found in P. Becket, M. J. Crimmin, M. H. Davis, Z. Spavold, Synlett, (1993), 137-138, incorporated herein by reference.

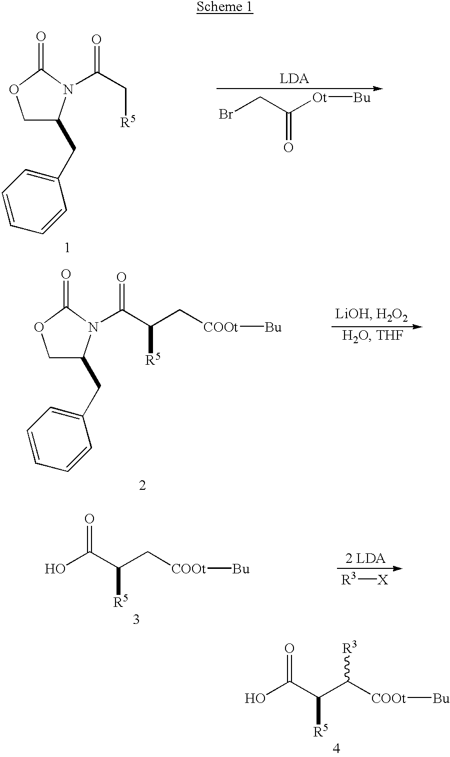

Diastereomerically pure succinate derivatives can be accessed using the chemistry outlined below, adapted from P. Becket, M. J. Crimmin, M. H. Davis, Z. Spavold, Synlett, (1993), 137-138 incorporated herein by reference. This reference provides the synthesis below to obtain compound 9. Compound 11 is used as an intermediate and is prepared from 9 by hydrogenation of the allyl group followed by coupling of 9-fluorenemethanol under standard conditions using DCC and DMAP in CH₂Cl₂. Deprotection of the tert-butyl ester is accomplished by treatment with 50% trifluoroacetic acid.

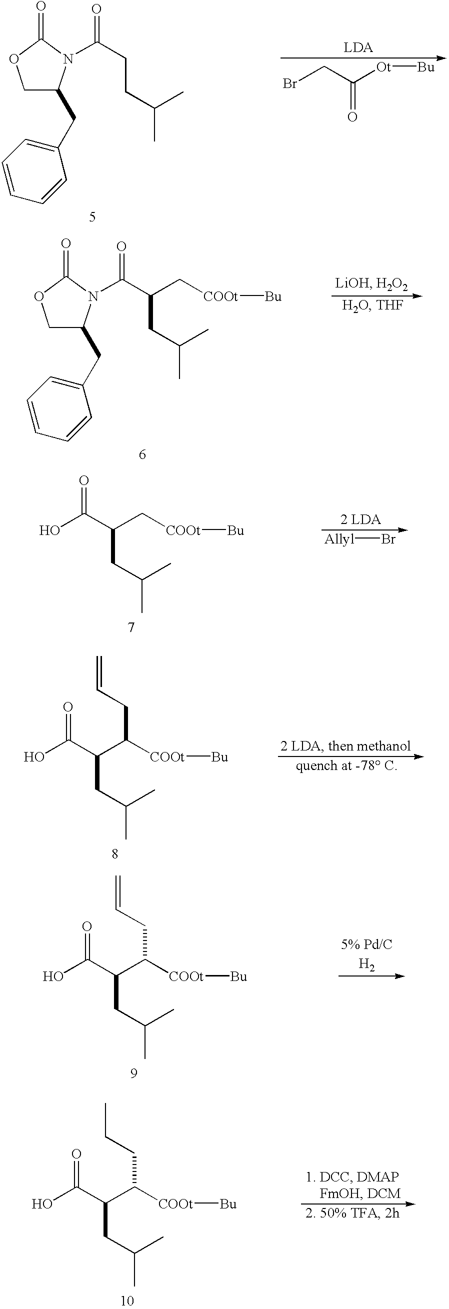

Succinates compounds wherein R³ and R^{3a} combine to form a cycloalkyl or carbocyclic moiety are known in the literature. For example, a dimethyl succinate having a 3-membered cyclopropyl can be formed by a thermal or photolytic decomposition of a methyl 3(carbomethoxymethyl)-1-pyrazoline-3-carboxylate. See Bull. Soc. Chim. Fr. (1971), (6), 2290-5. A succinic acid derivative containing a 4-membered cyclobutyl group can be formed by the method published in U.S. Pat. No. 3,828,025. A succinic acid derivative containing a 5-membered cyclopentyl group can be formed using the methods described in Le Moal, H. et al., Bull. Soc. Chim. Fr., 1964, 579-584; Borenstein, M. R., et al., Heterocycles, 22, 1984, 2433-2438. Other examples of derivatives of succinate X wherein ring C is a five-membered cyclopentyl group or a 6-membered cyclohexyl group have been employed as matrix metalloproteinase inhibitors. See Bioorg. Med. Chem. Lett. (1998), 8(12), 1443-1448; Robinson, R. P., et al., Bioorg. Med. Chem. Lett. (1996), 6(14), 1719-1724. It is understood that these references are only illustrative of the availability of some carbocyclic and cycloalkyl containing succinates, however numerous references are known in literature which provide preparations of other substituted carbocyclic and cycloalkyl containing succinates and their derivatives.

Scheme 2a illustrates one method for the introduction of a substitution on a carbon adjacent to the cyclic group in succinate X via a deprotonation followed by standard alkylation procedures known to one skilled in the art. Treatment of IX with a base followed by addition of an R⁵-LG, wherein LG is a leaving group such as a halide, mesylate, or a tosylate, and subsequent deprotection of the benzyl group by hydrogenation employing, for example, H₂ and Pd/C, would give the desired cyclic containing succinate X.

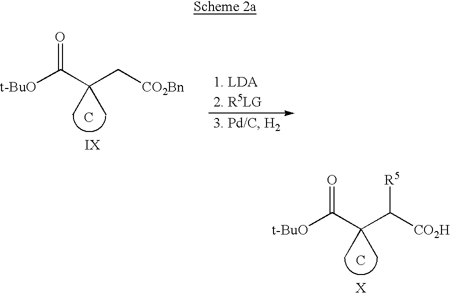

Additional methods useful for the preparation of succinate derivatives are known by those skilled in the art. Such references include McClure and Axt, Bioorganic & Medicinal Chemistry Letters, 8 (1998) 143-146; Jacobson and Reddy, Tetrahedron Letters, Vol 37, No. 46, 8263-8266 (1996); Pratt et al., SYMLETT, May 1998, p. 531; WO 97/18207; and WO 98/51665. The synthetic disclosures of WO97/18207 and WO 98/51665 are hereby incorporated by reference for the preparation of succinate derivatives. A further alkylation of disubstituted succinates such as 204 provides intermediates such as 205 useful as substrates suitable for cyclization reactions known to one skilled in the art, such as ring closing metathesis (RCM) reactions using Grubbs'catalyst as illustrated in Scheme 2b. It will be appreciated by those skilled in the art that the analogous preparation of other cyclization substrates and the use of alternative ring forming methodologies will provide access to carbo- analogs of intermediates 206 and 207. Examples of cyclized succinate derivatives can be found in U.S. Provisional Patent Application No. 60/208,536, incorporated herein by reference for the purpose of enabling cyclized succinate derivatives.

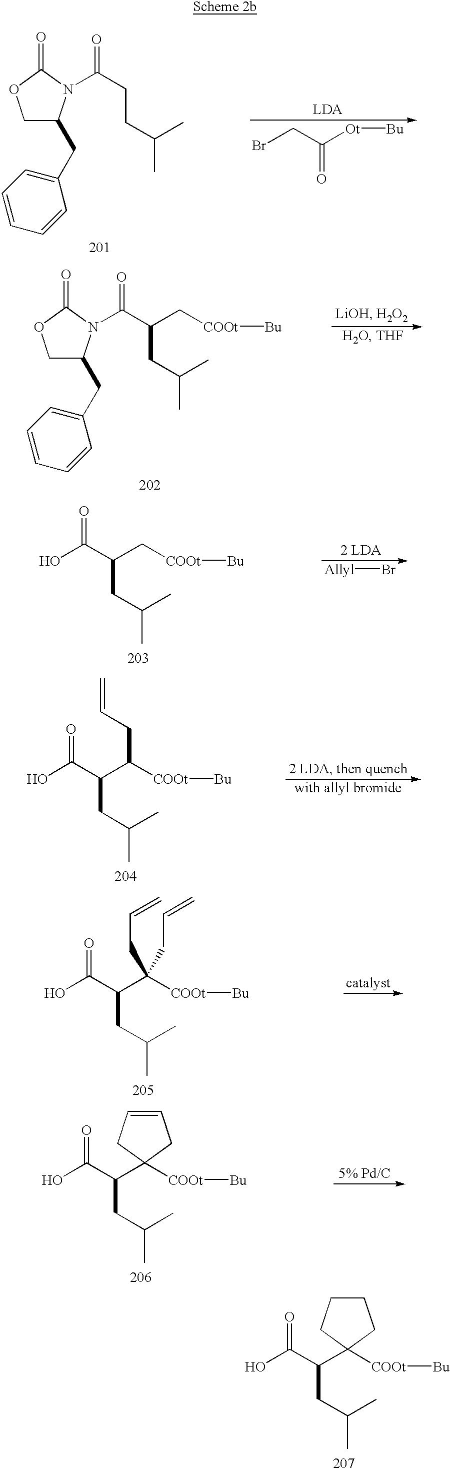

The compounds of the present invention may be synthesized using the succinates 4 and substituted heterocyclic amines as is shown in Scheme 3.

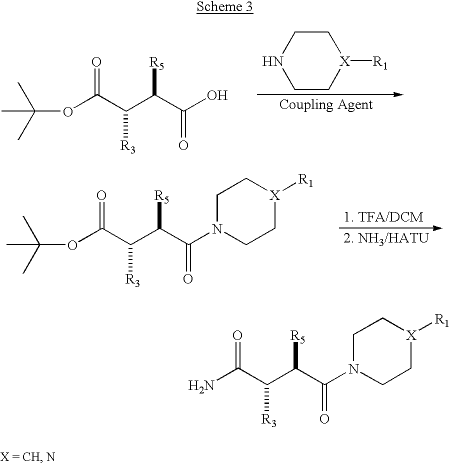

Additional examples may be prepared by adding a bifunctional amine followed by preparation of extended derivatives, as is demonstrated in Schemes 4 and 5, using piperazine and 4-piperidinone, respectively. In addition, these transformations may be carried out in parallel on solid phase starting with resin 13 of Scheme 8.

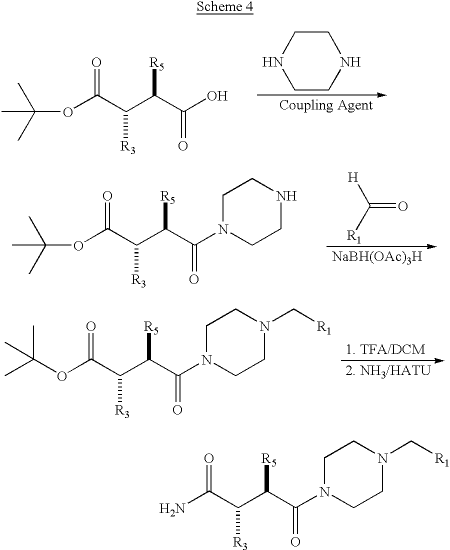

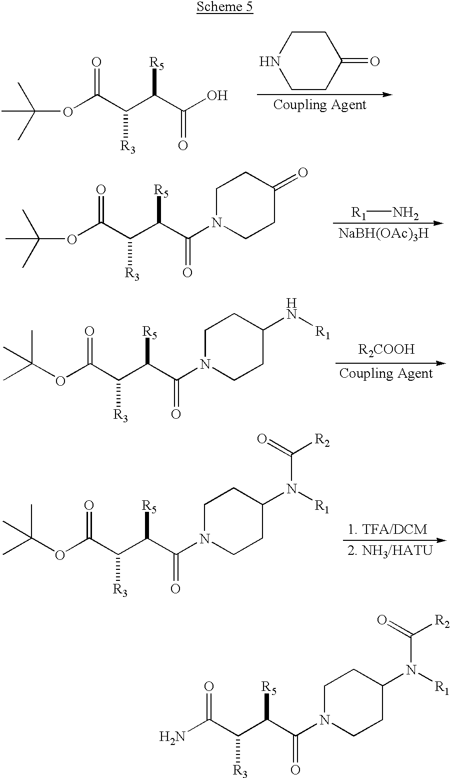

Additional examples of the compounds of claim 1 can be synthesized as is shown in Scheme 6, thus acylation of the Kenner Safety Catch linker (see Backes, B. J.; Virgilio, A. A.; Ellman, J. A. J.Amer.Chem.Soc. 1996, 118, 3055-3056, Backes, B. J.; Ellman, J. A. J.Amer.Chem.Soc. 1994, 116, 11171-11172, Backes, B. J.; Ellman, J. J.Org.Chem. 1999, 64, 2322-2330) with functionalized aminocyclic amides such as 24 provides the protected succinate 25. Deprotection followed by amide formation gives the succinamide which can be further elaborated upon cleavage to prepare a varitey of compounds such as 27 which are examples of the current invention.

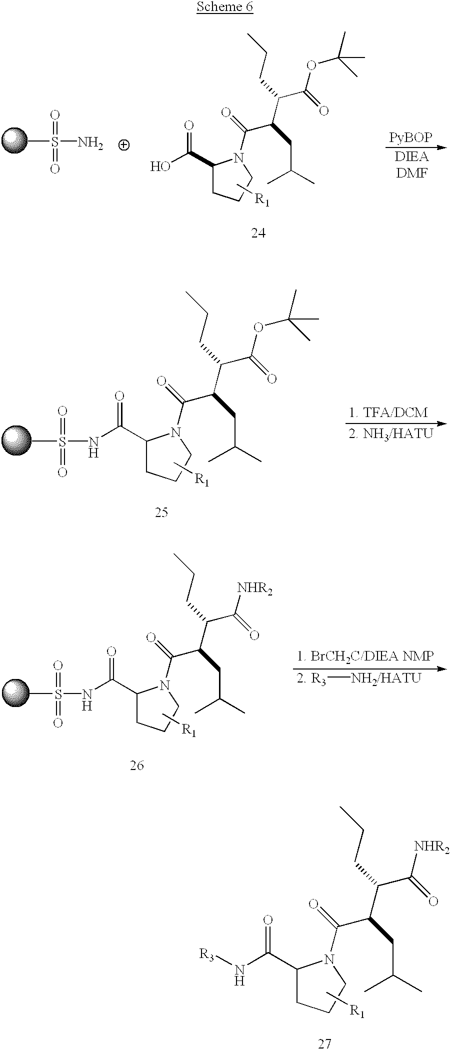

A wide variety of substituted piperidine derivatives are items of commerce. Additional derivatives are simply prepared starting from benzyl protected 3- or 4-piperidone as is shown in Scheme 7. Addition of lithium or Grignard reagents provides the functionalized piperidinols, which can be used to prepare compounds of this invention. Additionally, dehydration followed by deprotection of the benzyl group and hydrogenation of the olefin provides additional reagents, see for example references 1) V. Breu, H.-P. Maerki, E. Vieira and W. Wostl, WO 00/64873 A1 (2000); 2) B. Lohri and E. Vieira, WO 00/63173 A1 (2000); 3) R. Guller, A. Binggeli, V. Breu, D. Bur, W. Fischli, G. Hirth, C. Jenny, M. Kansy, F. Montavon, M. Muller, C. Oefner, H. Stadler, E. Vieira, M. Wilhelm, W. Wostl and H. P. Marki, Bioorg. Med. Chem. Lett., 9, 1403 (1999); and 4) E. Vieira, A. Binggeli, V. Breu, D. Bur, W. Fischli, R. Guller, G. Hirth, H. P. Marki, M. Muller, C. Oefner, M. Scalone, H. Stadler, M. Wilhelm and W. Wostl, Bioorg. Med. Chem. Lett., 9, 1397 (1999)

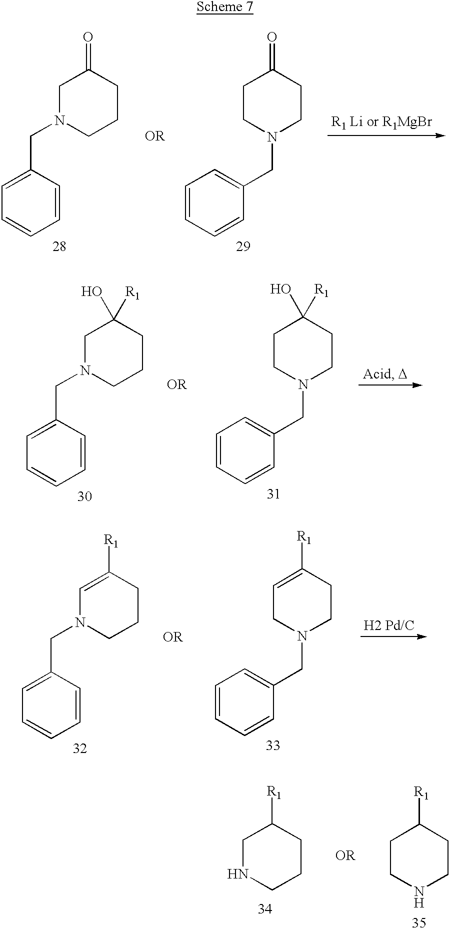

Additionally, the acid 11 can be coupled onto a variety of solid supports to initiate solid-phase parallel synthesis. The solid-phase synthesis of the compounds of claim 1 is shown in Scheme 8, where coupling of 11 to Peptide Amide Linker (PAL) resin (commercially available from Perkin Elmer Biosystems) produces the resin-bound succinamide 37. This coupling can be accomplished using a variety of coupling agents such as diisopropylcarbodiimide (DICI) with the additive 1-hydroxybenzotriazole (HOBt), HATU (O-(7-azabenzotriazol-1-yl)-1,1,3,3,-tetramethyluronium hexafluorophosphate) in the presence of a base such as diisopropylethylamine (DIEA) or triethylamine, PyBOP (benzotriazole-1-yl-oxy-tris-pyrrolidino-phosphonium hexafluorophosphate) or other coupling agents known to those skilled in the art (DICI with hydroxybenzotriazole is preferred). Preferred solvents for coupling reactions include N,N-dimethylformamide (DMF), N-methylpyrrolidinone (NMP), and dichloromethane (DCM).

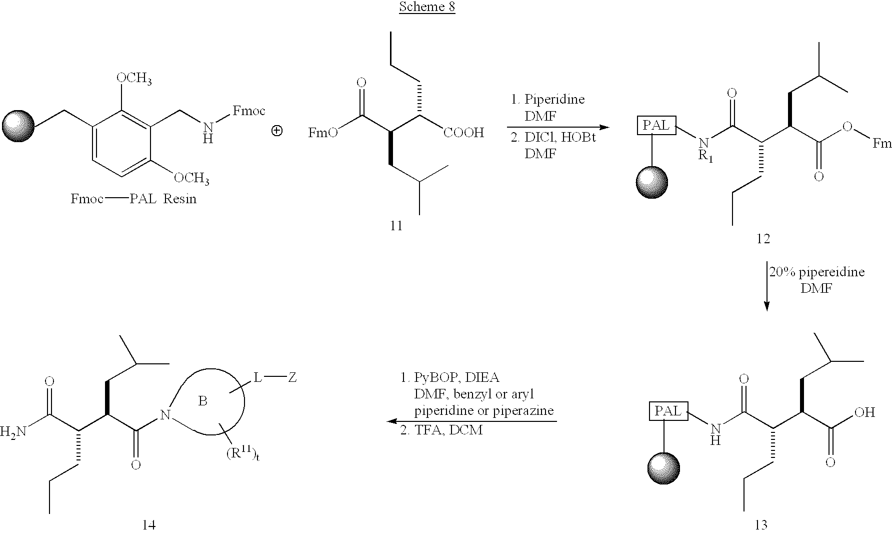

The fluorenylmethyl ester is removed from the compounds by treatment with piperidine and the resultant carboxylic acid can be reacted with a variety of animes to form the corresponding amides. Treatment with trifluoroacetic acid in dichloromethane then releases the desired compounds 14 from the solid support.

Additional methods useful for the preparation of succinate derivatives are known by those skilled in the art. Such references include, McClure and Axt, Bioorganic & Medicinal Chemistry Letters, 8 (1998) 143-146; Jacobson and Reddy, Tetrahedron Letters, Vol 37, No. 46, 8263-8266 (1996); Pratt et al., SYNLETT, May 1998, p. 531; WO 97/18207; and WO 98/51665. The synthetic disclosures of WO97/18207 and WO 98/51665 are hereby incorporated by reference.

### EXAMPLES

Succinate 10 of Scheme 2

Succinate 9 is prepared according to the literature procedure (P. Becket, M. J. Crimmin, M. H. Davis, Z. Spavold, Synlett, (1993), 137-138). Succinate 9 (17.8 g, 66 mmol) is dissolved in 250 mL of ethyl acetate and placed in a Parr shaker bottle. To the solution is added 890 mg of 5% palladium on carbon, and the bottle is pressurized to 40 psi with hydrogen gas and shaken for 2.5 h at rt. The hydrogen is removed and the palladium catalyst is removed by filtration through a pad of celite. Concentration of the ethyl acetate solution provides 17.5 g (98%) of succinate 10. No further purification is necessary. MS (M−H)⁺=271.

Succinate 11 of Scheme 1

Succinate 10 (6.3 g, 23.1 mmol) is dissolved in 125 mL of CH₂Cl₂ and 4.8 g (23.3 mmol) of dicyclohexylcarbodiimide is added. The solution is stirred at rt for 30 min and then 4.6 g (23.4 mmol) of 9-fluorenemethanol is added followed by 122 mg (1 mmol) of 4-dimethylaminopyridine. After 5 h of stirring at rt, the reaction solution was diluted with an additional 100 mL of CH₂Cl₂ and filtered through a pad of celite to remove precipitated dicyclohexylurea. The solution was then washed 3× with 50 mL of a 1N HCl solution, 3× with 50 mL of a saturated sodium bicarbonate solution, and 2× with 50 mL of brine. The crude product was dried over MgSO₄ and concentrated onto 15 g of silica gel. Chromatography eluting with a gradient of 2.5% to 5% ethyl acetate/hexanes provided 6.4 g (61%) of the diester as an oil. The purified diester (6.4 g 14.2 mmol) is then dissolved in 25 mL of CH₂Cl₂, 25 mL of trifluoroacetic acid is added, and the reaction solution is stirred at rt for 2 h. The reaction solution is directly concentrated in vacuo to an oil which is then redissolved in 25 mL of toluene and reconcentrated, followed by drying in vacuo to provide 6.3 g (98%) of the desired succinate 9 as an oil which solidifies on standing. MS (M+Na)⁺=471, (M+2Na)⁺=439.

General Procedure for Solid-phase Synthesis According to Scheme 8

General: The phrase “washed under standard conditions” when applied to a resin refers to rinsing the resin as a slurry three times in DMF followed by 3 times in methanol followed by three times in dichloromethane using approximately 10 mL of solvent per gram of resin.

Resin 37 of Scheme 8: Commercial Fmoc-PAL resin (Perkin Elmer Biosystems) (9 grams, 0.42 mmol/g, 3.78 mmol) is washed for 20 min with 3×50 mL of 20% piperidine in DMF. The resulting free amine resin is then washed under standard conditions. The resin is then slurried in 100 mL of DMF and and 4.47 grams (11.34 mmol) of succinate 11 is then added, followed by HOBt (1.74 g, 11.34 mmol) and diisopropylcarbodiimide (1.82 mL, 11.34 mmol). The resin is placed on a shaker table for 16 h and then washed under standard conditions and dried in vacuo.

Resin 38 of Scheme 8: Resin 12 of scheme 3 is washed for 20 min with 3×50 mL of 20% piperidine in DMF. The resulting free carboxylic acid resin is then washed under standard conditions.

Products 39 of Scheme 8: Six grams of resin is suspended in a 2:3 mixture of DMF and CH₂Cl₂ and pipetted into 118 of the wells of two commercial polyfiltronics 96-well filter blocks, approximately 50 mg of resin per well. The solvents are removed by filtration, and 200 μL of DMF is added to each reaction well, followed by 110 μL of a 1 M solution of the desired amine in DMF. A stock solution of PyBOP (6.56 g, 12.6 mmol) dissolved in 24 mL of DMF is then prepared, and 200 μL of this solution (0.10 mmol) is added to each well. Diisopropylethylamine (0.21 mmol, 36.5 μL) is then added to each well and the reaction block is sealed and mixed on a shaker table for 16 h. The plates are then washed under standard conditions. The compounds are then cleaved from the solid support employing 1 mL of a 95:5 trifluoroacetic acid/triethylsilane solution for 3 h. The cleavage solution is drained from the well and the resin is washed with an additional 0.5 mL of DCM and the combined filtrates are concentrated. The samples are redissolved in 1 mL of methanol and reconcentrated to remove any volatile impurities.

### Examples 1-106

For each reagent listed in Table 1, the corresponding product 39 was prepared. The products of Examples 1-106 were verified by the presence of the desired compound in ESI MS (M+H⁺ or M+Na⁺).

### Example 1

3(R)-(4-Benzo[1,3]dioxol-5-ylmethyl-piperazine-1-carbonyl)-5-methyl-2(S)-propyl-hexanoic acid amide. MS [M+H]⁺ 418.1.

### Example 2

5-Methyl-3(R)-(piperazine-1-carbonyl)-2(S)-propyl-hexanoic acid amide. MS [M+H]⁺ 284.1.

### Example 3

5-Methyl-3(R)-(4-phenyl-piperazine-1-carbonyl)-2(S)-propyl-hexanoic acid amide. MS [M+H]⁺ 360.1.

### Example 4

3(R)-[4-(2-Methoxy-phenyl)-piperazine-1-carbonyl]-5-methyl-2(S)-propyl-hexanoic acid amide. MS [M+H]⁺ 390.1.

### Example 5

5-Methyl-2(S)-propyl-3(R)-[4-(3-trifluoromethyl-phenyl)-piperazine-1-carbonyl]-hexanoic acid amide. MS [M+H]⁺ 428.1.

### Example 6

3(R)-[4-(4-Fluoro-phenyl)-piperazine-1-carbonyl]-5-methyl-2(S)-propyl-hexanoic acid amide. MS [M+H]⁺ 378.1.

### Example 7

5-Methyl-3(R)-[4-(4-nitro-phenyl)-piperazine-1-carbonyl]-2(S)-propyl-hexanoic acid amide. MS [M+H]⁺ 405.1.

### Example 8

5-Methyl-3(R)-(4-methyl-piperazine-1-carbonyl)-2(S)-propyl-hexanoic acid amide. MS [M+H]⁺ 298.1.

### Example 9

3(R)-(4-Benzyl-piperazine-1-carbonyl)-5-methyl-2(S)-propyl-hexanoic acid amide. MS [M+H]⁺ 374.1

### Example 10

3(R)-[4-(2-Hydroxy-ethyl)-piperazine-1-carbonyl]-5-methyl-2(S)-propyl-hexanoic acid amide. MS [M+H]⁺ 328.1

### Example 11

5-Methyl-2(S)-propyl-3(R)-(4-pyridin-2-yl-piperazine-1-carbonyl)-hexanoic acid amide. MS [M+H]⁺ 361.1.

### Example 12

3(R)-[4-(2-Chloro-phenyl)-piperazine-1-carbonyl]-5-methyl-2(S)-propyl-hexanoic acid amide. MS [M+H]⁺ 394.1.

### Example 13

5-Methyl-3(R)-(3-methyl-4-phenyl-piperazine-1-carbonyl)-2(S)-propyl-hexanoic acid amide. MS [M+H]⁺ 374.1.

### Example 14

3(R)-[4-(4-Methoxy-phenyl)-3-methyl-piperazine-1-carbonyl]-5-methyl-2(S)-propyl-hexanoic acid amide. MS [M+H]⁺ 404.2.

### Example 15

5-Methyl-2(S)-propyl-3(R)-(4-p-tolyl-piperazine-1-carbonyl)-hexanoic acid amide. MS [M+H]⁺ 374.1.

### Example 16

3(R)-[4-(3-Methoxy-phenyl)-piperazine-1-carbonyl]-5-methyl-2(S)-propyl-hexanoic acid amide. MS [M+H]⁺ 390.1.

### Example 17

[4-(3(S)-Carbamoyl-2(R)-isobutyl-hexanoyl)-piperazin-1-yl]-acetic acid ethyl ester. MS [M+H]⁺ 370.1.

### Example 18

5-Methyl-3(R)-(3-methyl-4-m-tolyl-piperazine-1-carbonyl)-2(S)-propyl-hexanoic acid amide. MS [M+H]⁺ 388.2.

### Example 19

3(R)-(4-Acetyl-piperazine-1-carbonyl)-5-methyl-2(S)-propyl-hexanoic acid amide. MS [M+H]⁺ 326.1.

### Example 20

3(R)-(4-Ethyl-piperazine-1-carbonyl)-5-methyl-2(S)-propyl-hexanoic acid amide. MS [M+H]⁺ 312.2.

### Example 21

5-Methyl-3(R)-[4-(3-phenyl-allyl)-piperazine-1-carbonyl]-2(S)-propyl-hexanoic acid amide. MS [M+H]⁺ 400.2.

### Example 22

3(R)-{4-[2-(2-Hydroxy-ethoxy)-ethyl]-piperazine-1-carbonyl}-5-methyl-2(S)-propyl-hexanoic acid amide. MS [M+H]⁺ 372.2

### Example 23

5-Methyl-2(S)-propyl-3(R)-(4-{2-[(pyridin-2-ylmethyl)-amino]-ethyl}-piperazine-1-carbonyl)-hexanoic acid amide. MS [M+H]⁺ 418.1.

### Example 24

3(R)-[4-(5-Chloro-2-methyl-phenyl)-piperazine-1-carbonyl]-5-methyl-2(S)-propyl-hexanoic acid amide. MS [M+H]⁺ 408.1.

### Example 25

5-Methyl-3 (R)-(octahydro-quinoxaline-1-carbonyl)-2(S)-propyl-hexanoic acid amide. MS [M+H]⁺ 338.5.

### Example 26

5-Methyl-3(R)-(4-(2-keto-1-benzimidazolinyl)-piperidine-1-carbonyl)-2(S)-propyl-hexanoic acid amide. MS [M+H]⁺ 415.1.

### Example 27

5-Methyl-3(R)-(2-methyl-piperidine-1-carbonyl)-2(S)-propyl-hexanoic acid amide. MS [M+H]⁺ 297.1.

### Example 28

1-(3(S)-Carbamoyl-²(R)-isobutyl-hexanoyl)-piperidine-2-carboxylic acid ethyl ester. MS [M+H]⁺ 355.1.

### Example 29

3(R)-(2-Hydroxymethyl-piperidine-1-carbonyl)-5-methyl-2(S)-propyl-hexanoic acid amide. MS [M+H]⁺ 313.1.

### Example 30

1-(3(S)-Carbamoyl-2(R)-isobutyl-hexanoyl)-piperidine-3-carboxylic acid amide. MS [M+H]⁺ 326.1.

### Example 31

1-(3(S)-Carbamoyl-2(R)-isobutyl-hexanoyl)-piperidine-3-carboxylic acid. MS [M+H]⁺ 327.1.

### Example 32

1-(3(S)-Carbamoyl-2(R)-isobutyl-hexanoyl)-piperidine-3-carboxylic acid ethyl ester. MS [M+H]⁺ 355.1.

### Example 33

1-(3(S)-Carbamoyl-2(R)-isobutyl-hexanoyl)-piperidine-3-carboxylic acid diethylamide. MS [M+H]⁺ 382.2.

### Example 34

3(R)-(3,5-Dimethyl-piperidine-1-carbonyl)-5-methyl-2(S)-propyl-hexanoic acid amide. MS [M+H]⁺ 311.1.

### Example 35

3(R)-(3-Hydroxymethyl-piperidine-1-carbonyl)-5-methyl-2(S)-propyl-hexanoic acid amide. MS [M+H]⁺ 313.1.

### Example 36

3(R)-(4-Hydroxy-piperidine-1-carbonyl)-5-methyl-2(S)-propyl-hexanoic acid amide. MS [M+H]⁺ 299.1.

### Example 37

1-(3(S)-Carbamoyl-2(R)-isobutyl-hexanoyl)-piperidine-4-carboxylic acid ethyl ester. MS [M+H]⁺ 355.1.

### Example 38

5-Methyl-3(R)-(4-methyl-piperidine-1-carbonyl)-2(S)-propyl-hexanoic acid amide. MS [M+H]⁺ 297.1.

### Example 39

3(R)-(4-Benzyl-piperidine-1-carbonyl)-5-methyl-2(S)-propyl-hexanoic acid amide. MS [M+H]⁺ 373.1.

### Example 40

3(R)-(4-Aminomethyl-piperidine-1-carbonyl)-5-methyl-2(S)-propyl-hexanoic acid amide. MS [M+H]⁺ 312.1.

### Example 41

3(R)-[4-(2-Hydroxy-ethyl)-piperidine-1-carbonyl]-5-methyl-2(S)-propyl-hexanoic acid amide. MS [M+H]⁺ 327.1.

### Example 42

3(R)-([1,4′]Bipiperidinyl-1′-carbonyl)-5-methyl-2(S)-propyl-hexanoic acid amide. MS [M+H]⁺ 366.2.

### Example 43

5-Methyl-3(R)-(octahydro-quinoline-1-carbonyl)-2(S)-propyl-hexanoic acid amide. MS [M+H]⁺ 337.1.

### Example 44

5-Methyl-3(R)-[4-(2-piperidin-4-yl-ethyl)-piperidine-1-carbonyl]-2(S)-propyl-hexanoic acid amide. MS [M+H]⁺ 394.2.

### Example 45

3(R)-(3-Hydroxy-piperidine-1-carbonyl)-5-methyl-2(S)-propyl-hexanoic acid amide. MS [M+H]⁺ 299.1.

### Example 46

3(R)-{2-[2-(3,5-Bis-trifluoromethyl-phenylamino)-ethyl]-piperidine-1-carbonyl}-5-methyl-2(S)-propyl-hexanoic acid amide. MS [M+H]⁺ 538.1.

### Example 47

3(R)-{2-[2-(4-Isopropyl-phenylamino)-ethyl]-piperidine-1-carbonyl}-5-methyl-2(S)-propyl-hexanoic acid amide. MS [M+H]⁺ 441.2.

### Example 48

3(R)-(4-Dimethylamino-piperidine-1-carbonyl)-5-methyl-2(S)-propyl-hexanoic acid amide. MS [M+H]⁺ 326.2.

### Example 49

5-Methyl-3(R)-[4-(3-phenyl-propyl)-piperidine-1-carbonyl]-2(S)-propyl-hexanoic acid amide. MS [M+H]⁺ 401.2.

### Example 50

5-Methyl-2(S)-propyl-3(R)-(4-propyl-piperidine-1-carbonyl)-hexanoic acid amide. MS [M+H]⁺ 325.2.

### Example 51

5-Methyl-3(R)-(4-phenyl-4-propionyl-piperidine-1-carbonyl)-2(S)-propyl-hexanoic acid amide. MS [M+H]⁺ 415.1.

### Example 52

1-(3(S)-Carbamoyl-2(R)-isobutyl-hexanoyl)-4-dimethylamino-piperidine-4-carboxylic acid amide. MS [M+H]⁺ 369.2.

### Example 53

5-Methyl-2(S)-propyl-3(R)-(4-pyrrolidin-1-yl-piperidine-1-carbonyl)-hexanoic acid amide. MS [M+H]⁺ 352.2.

### Example 54

1-(3(S)-Carbamoyl-2(R)-isobutyl-hexanoyl)-piperidine-4-carboxylic acid amide. MS [M+H]⁺ 326.1.

### Example 55

5-Methyl-3(R)-(piperidine-1-carbonyl)-2(S)-propyl-hexanoic acid amide. MS [M+H]⁺ 327.1.

### Example 56

5-Methyl-3(R)-(2-piperidin-1-ylmethyl-piperidine-1-carbonyl)-2(S)-propyl-hexanoic acid amide. MS [M+H]⁺ 380.2.

### Example 57

1-(3(S)-Carbamoyl-2(R)-isobutyl-hexanoyl)-4-phenylamino-piperidine-4-carboxylic acid amide. MS [M+H]⁺ 417.1.

### Example 58

3(R)-{4-[(2-Amino-ethylamino)-methyl]-piperidine-1-carbonyl}-5-methyl-2(S)-propyl-hexanoic acid amide. MS [M+H]⁺ 355.2.

### Example 59

1-(3(S)-Carbamoyl-2(R)-isobutyl-hexanoyl)-4-cyclohexylamino-piperidine-4-carboxylic acid amide. MS [M+H]⁺ 423.2.

### Example 60

1-(3(S)-Carbamoyl-2(R)-isobutyl-hexanoyl)-4-ethylamino-piperidine-4-carboxylic acid amide. MS [M+H]⁺ 369.2.

### Example 61

5-Methyl-3(R)-(3-methyl-3-phenyl-piperidine-1-carbonyl)-2(S)-propyl-hexanoic acid amide. MS [M+H]⁺ 373.1.

### Example 62

3(R)-[3-Hydroxy-4-(3-trifluoromethyl-phenyl)-piperidine-1-carbonyl]-5-methyl-2(S)-propyl-hexanoic acid amide. MS [M+H]⁺ 443.1.

### Example 63

3(R)-(3-Bromo-piperidine-1-carbonyl)-5-methyl-2(S)-propyl-hexanoic acid amide. MS [M+H]⁺ 361.3.

### Example 64

3(R)-(3-Hydroxy-piperidine-1-carbonyl)-5-methyl-2(S)-propyl-hexanoic acid amide. MS [M+H]⁺ 298.4.

### Example 65

3(R)-[4-(4-Chloro-phenyl)-piperazine-1-carbonyl]-5-methyl-2(S)-propyl-hexanoic acid amide. MS [M+H]⁺ 394.1.

### Example 66

3(R)-[4-(2-Ethoxy-phenyl)-piperazine-1-carbonyl]-5-methyl-2(S)-propyl-hexanoic acid amide. MS [M+H]⁺ 404.6.

### Example 67

3(R)-[4-(4-Fluoro-phenyl)-piperazine-1-carbonyl]-5-methyl-2(S)-propyl-hexanoic acid amide. MS [M+H]⁺ 378.1.

### Example 68

3(R)-[4-(2,4-Dimethyl-phenyl)-piperazine-1-carbonyl]-5-methyl-2(S)-propyl-hexanoic acid amide. MS [M+H]⁺ 388.2.

### Example 69

3(R)-[4-(4-Chloro-phenyl)-3-methyl-piperazine-1-carbonyl]-5-methyl-2(S)-propyl-hexanoic acid amide. MS [M+H]⁺ 408.1.

### Example 70

3(R)-[4-(3,4-Dichloro-phenyl)-piperazine-1-carbonyl]-5-methyl-2(S)-propyl-hexanoic acid amide. MS [M+H]⁺ 430.0.

### Example 71

3(R)-[4-(3,4-Dimethyl-phenyl)-piperazine-1-carbonyl]-5-methyl-2(S)-propyl-hexanoic acid amide. MS [M+H]⁺ 388.2.

### Example 72

3(R)-[4-(2,6-Dimethyl-phenyl)-piperazine-1-carbonyl]-5-methyl-2(S)-propyl-hexanoic acid amide. MS [M+H]⁺ 388.2.

### Example 73

3(R)-[4-(3-Chloro-phenyl)-piperazine-1-carbonyl]-5-methyl-2(S)-propyl-hexanoic acid amide. MS [M+H]⁺ 394.1.

### Example 74

3(R)-[4-(2-Fluoro-phenyl)-piperazine-1-carbonyl]-5-methyl-2(S)-propyl-hexanoic acid amide. MS [M+H]⁺ 378.1.

### Example 75

3(R)-[4-(2-Chloro-phenyl)-piperazine-1-carbonyl]-5-methyl-2(S)-propyl-hexanoic acid amide. MS [M+H]⁺ 394.1.

### Example 76

3(R)-[4-(2-Nitro-phenyl)-piperazine-1-carbonyl]-5-methyl-2(S)-propyl-hexanoic acid amide. MS [M+H]⁺ 405.1.

### Example 77

3(R)-[4-(2-Methyl-phenyl)-piperazine-1-carbonyl]-5-methyl-2(S)-propyl-hexanoic acid amide. MS [M+H]⁺ 374.1.

### Example 78

3(R)-[4-(2-Ethyl-phenyl)-piperazine-1-carbonyl]-5-methyl-2(S)-propyl-hexanoic acid amide. MS [M+H]⁺ 388.2.

### Example 79

3(R)-[4-(3-Methyl-phenyl)-piperazine-1-carbonyl]-5-methyl-2(S)-propyl-hexanoic acid amide. MS [M+H]⁺ 374.1.

### Example 80

3(R)-[4-(4-Chloro-3-trifluoromethyl-phenyl)-piperazine-1-carbonyl]-5-methyl-2(S)-propyl-hexanoic acid amide. MS [M+H]⁺ 462.0.

### Example 81

3(R)-[4-(4-Methyl-phenyl)-piperazine-1-carbonyl]-5-methyl-2(S)-propyl-hexanoic acid amide. MS [M+H]⁺ 374.1.

### Example 82

5-Methyl-2(S)-propyl-3(R)-(4-pyrimidin-2-yl-piperazine-1-carbonyl)-hexanoic acid amide. MS [M+H]⁺ 361.1.

### Example 83

3(R)-[4-(2,3-Dimethyl-phenyl)-piperazine-1-carbonyl]-5-methyl-2(S)-propyl-hexanoic acid amide. MS [M+H]⁺ 388.2.

### Example 84

5-Methyl-2(S)-propyl-3(R)-(4-pyridin-4-yl-piperazine-1-carbonyl)-hexanoic acid amide. MS [M+H]⁺ 361.1.

### Example 85

3(R)-[4-(3,5-Dichloro-phenyl)-piperazine-1-carbonyl]-5-methyl-2(S)-propyl-hexanoic acid amide. MS [M+H]⁺ 428.1.

### Example 86

5-Methyl-2(S)-propyl-3(R)-[4-(4-trifluoromethyl-phenyl)-piperazine-1-carbonyl]-hexanoic acid amide. MS [M+H]⁺ 428.1.

### Example 87

5-Methyl-2(S)-propyl-3(R)-(4-pyrazin-2-yl-piperazine-1-carbonyl-carbonyl)-hexanoic acid amide. MS [M+H]⁺ 362.1.

### Example 88

3(R)-[4-(2-Cyano-phenyl)-piperazine-1-carbonyl]-5-methyl-2(S)-propyl-hexanoic acid amide. MS [M+H]⁺ 385.1.

### Example 89

3(R)-[4-(2,4-Dimethoxy-phenyl)-piperazine-1-carbonyl]-5-methyl-2(S)-propyl-hexanoic acid amide. MS [M+H]⁺ 420.1.

### Example 90

3(R)-(4-Benzo[1,3]dioxol-5-yl-piperazine-1-carbonyl)-5-methyl-2(S)-propyl-hexanoic acid amide. MS [M+H]⁺ 404.1.

### Example 91

5-Methyl-3(R)-(3-methyl-4-p-tolyl-piperazine-1-carbonyl)-2(S)-propyl-hexanoic acid amide. MS [M+H]⁺ 388.2.

### Example 92

3(R)-[4-(3-Methoxy-phenyl)-piperazine-1-carbonyl]-5-methyl-2(S)-propyl-hexanoic acid amide. MS [M+H]⁺ 390.1.

### Example 94

3(R)-[4-(4-Chloro-3-trifluoromethyl-phenyl)-4-hydroxy-piperidine-1-carbonyl]-5-methyl-2(S)-propyl-hexanoic acid amide. MS [M+H]⁺ 477.0.

### Example 96

3(R)-{4-[(4-Chloro-phenyl)-phenyl-methyl]-piperazine-1-carbonyl}-5-methyl-2(S)-propyl-hexanoic acid amide. MS [M+H]⁺ 485.1.

### Example 97

5-Methyl-3(R)-[2-(1-methyl-pyrrolidin-2-ylmethyl)-piperidine-1-carbonyl]-2(S)-propyl-hexanoic acid amide. MS [M+H]⁺ 380.0.

### Example 98

5-Methyl-2(S)-propyl-3(R)-[4-(5-trifluoromethyl-pyridin-2-yl)-piperazine-1-carbonyl]-hexanoic acid amide. MS [M+H]⁺ 429.1.

### Example 99

5-Methyl-2(S)-propyl-3(R)-[4-(3-trifluoromethyl-pyridin-2-yl)-piperazine-1-carbonyl]-hexanoic acid amide. MS [M+H]⁺ 428.492.

### Example 100

3(R)-(4-Cyano-4-phenyl-piperidine-1-carbonyl)-5-methyl-2(S)-propyl-hexanoic acid amide. MS [M+H]⁺ 384.1.

### Example 101

3(R)-(4-Hydroxy-4-phenyl-piperidine-1-carbonyl)-5-methyl-2(S)-propyl-hexanoic acid amide. MS [M+H]⁺ 375.1.

### Example 102

5-Methyl-2(S)-propyl-3(R)-(4-pyrrolidin-1-yl-piperidine-1-carbonyl)-hexanoic acid amide. MS [M+H]⁺ 352.2.

### Example 103

3(R)-(4-Acetyl-4-phenyl-piperidine-1-carbonyl)-5-methyl-2(S)-propyl-hexanoic acid amide. MS [M+H]⁺ 401.1.

### Example 104

3(R)-[4-(4-Chloro-phenyl)-4-hydroxy-piperidine-1-carbonyl]-5-methyl-2(S)-propyl-hexanoic acid amide. MS [M+H]⁺ 392.1.

### Example 105

3(R)-[4-(3-Hydroxy-propyl)-piperazine-1-carbonyl]-5-methyl-2(S)-propyl-hexanoic acid amide. MS [M+H]⁺ 342.1.

### Example 106

3(R)-[4-(3-Chloro-phenyl)-piperazine-1-carbonyl]-5-methyl-2(S)-propyl-hexanoic acid amide. MS [M+H]⁺ 395.1.

TABLE 1

| | |
|---|---|
| | 39 |
| 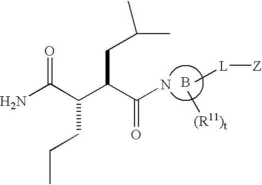 | |

| | Reagent Name | Molecular Weight |
|---|---|---|
| Example | for (un)substituted Ring B | of Product |
| 1 | 1-piperonylpiperazine | 418.1 |
| 2 | piperazine | 284.1 |
| 3 | 1-phenylpiperazine | 360.1 |
| 4 | 1-(2-methoxyphenyl)piperazine | 390.1 |
| 5 | n-(3-trifluoromethylphenyl)piperazine | 428.1 |
| 6 | 1-(4-fluorophenyl)piperazine | 378.1 |
| 7 | 1-(4-nitrophenyl)piperazine | 405.1 |
| 8 | 1-methylpiperazine | 298.1 |
| 9 | 1-benzylpiperazine | 374.1 |
| 10 | n-(2-hydroxyethyl)piperazine | 328.1 |
| 11 | 1-(2-pyridyl)piperazine | 361.1 |
| 12 | 1-(2-chlorophenyl)-piperazine, monohydrochloride | 394.1 |
| 13 | 2-methyl-1-phenylpiperazine | 374.1 |
| 14 | 1-(4-methoxyphenyl)-2-methylpiperazine | 404.2 |
| 15 | 1-(p-tolyl)-piperazine dihydrochloride | 374.1 |
| 16 | 1-(3-methoxyphenyl)piperazine dihydrochloride | 390.1 |
| 17 | n-(carboethoxymethyl)piperazine | 370.1 |
| 18 | 2-methyl-1-(3-methylphenyl)piperazine | 388.2 |
| 19 | 1-acetylpiperaine | 326.1 |
| 20 | n-ethylpiperazine | 312.2 |
| 21 | trans-1-cinnamylpiperazine | 400.2 |
| 22 | 1-hydroxyethylethloxypiperazine | 372.2 |
| 23 | 1-(2-(2-pyridylmethylamino)-ethyl)-piperazine | 418.1 |
| 24 | 1-(5-chloro-ortho-tolyl)-piperazine | 408.1 |
| 25 | perhydroquinoxaline | 338.5 |
| 26 | 4-(2-keto-1-benzimidazolinyl)piperidine | 415.1 |
| 27 | 2-methylpiperidine | 297.1 |
| 28 | ethyl pipecolinate | 355.1 |
| 29 | 2-piperidinemethanol | 313.1 |
| 30 | nipecotamide | 326.1 |
| 31 | nipecotic acid | 327.1 |
| 32 | ethyl nipecotate | 355.1 |
| 33 | n,n-diethylnipecotamide | 382.2 |
| 34 | 3,5-dimethylpiperidine | 311.1 |
| 35 | 3-piperidinemethanol | 313.1 |
| 36 | 4-hydroxypiperidine | 299.1 |
| 37 | ethyl isonipecotate | 355.1 |
| 38 | 4-methylpiperidine | 297.1 |
| 39 | 4-benzylpiperidine | 373.1 |
| 40 | 4-(aminomethyl)piperidine | 312.1 |
| 41 | 4-piperidineethanol | 327.1 |
| 42 | 4-piperidinopiperidine | 366.2 |
| 43 | decahydroquinoline | 337.1 |
| 44 | 4,4′-ethylenedipiperidine 2HCl | 394.2 |
| 45 | 3-hydroxypiperidine | 299.1 |
| 46 | N-[2-(2-piperidyl)ethyl]-3,5-bis-(trifluoromethyl)aniline | 538.1 |
| 47 | 2-(2-(4-isopropylanilino)ethyl)-piperidine | 444.2 |
| 48 | 4-(dimethylamino)-piperidine | 326.2 |
| 49 | 4-(3-phenylpropyl)-piperidine | 401.2 |
| 50 | 4-n-propylpiperidine | 325.2 |
| 51 | 4-phenyl-4-propionylpiperidine HCl | 415.1 |
| 52 | 4-carbamoyl-4-(dimethylamino)piperidine dihydrochloride | 369.2 |
| 53 | 4-(1-pyrrolidinyl)piperidine | 352.2 |
| 54 | isonipecotamide | 326.1 |
| 55 | d1-pipecolinic acid | 327.1 |
| 56 | 2-(piperidinomethyl)-piperidine | 380.2 |
| 57 | 4-anilino-4-carbamylpiperidine | 417.1 |
| 58 | n-(4-piperidylmethyl)-ethylenediamine | 355.2 |
| 59 | 4-(cyclohexylamino)-isonipecotamide | 423.2 |
| 60 | 4-(ethylamino)-isonipecotamide | 369.2 |
| 61 | 3-methyl-3-phenylpiperidine | 373.1 |
| 62 | 4-(3-(trifluoromethyl)phenyl)-3-piperidinol HCl | 443.1 |
| 63 | 4-bromopiperidine HBr | 361.3 |
| 64 | (r)-(+)-3-hydroxypiperidine HCl | 298.4 |
| 65 | 1-(4-chlorophenyl)piperazine 2HCl | 394.1 |
| 66 | 1-(2-ethoxyphenyl)piperazine HCl | 404.6 |
| 67 | 1-(4-fluorophenyl)piperazine 2HCl | 378.1 |
| 68 | 1-(2,4-dimethylphenyl)piperazine | 388.2 |
| 69 | 1-(4-chlorophenyl)-2-methylpiperazine | 408.1 |
| 70 | n-(3,4-dichlorophenyl)piperazine | 430.0 |
| 71 | 1-(3,4-dimethylphenyl)piperazine | 388.2 |
| 72 | 1-(2,6-dimethylphenyl)piperazine | 388.2 |
| 73 | 1-(3-chlorophenyl)piperazine HCl | 394.1 |
| 74 | 1-(2-fluorophenyl)piperazine | 378.1 |
| 75 | 1-(2-chlorophenyl)piperazine | 394.1 |
| 76 | 1-(2-nitrophenyl)piperazine | 405.1 |
| 77 | 1-(2-methylphenyl)piperazine | 374.1 |
| 78 | 1-(2-ethylphenyl)piperazine | 388.2 |
| 79 | 1-(3-methylphenyl)piperazine | 374.1 |
| 80 | 1-(3-trifluoromethyl-4-chlorophenyl)-piperazine | 462.0 |
| 81 | 1-(4-methylphenyl)piperazine | 374.1 |
| 82 | 1-(2-pyrimidyl)piperazine | 362.1 |
| 83 | 1-(2,3-dimethylphenyl)piperazine | 388.2 |
| 84 | 1-(4-pyridyl)piperazine | 361.1 |
| 85 | 1-(3,5-dichlorophenyl)piperazine | 428.1 |
| 86 | 1-(4-trifluoromethylphenyl)piperazine | 428.1 |
| 87 | 1-(2-pyrazinyl)piperazine | 362.1 |
| 88 | 1-(2-cyanophenyl)piperazine | 385.1 |
| 89 | 1-(2,4-dimethoxyphenyl)piperazine | 420.1 |
| 90 | 1-(3,4-methylenedioxyphenyl)piperazine hydrochloride | 404.1 |
| 91 | 1-(4-methylphenyl)-2-methylpiperazine | 388.2 |
| 92 | 1-(3-methoxyphenyl)piperazine 2HCl | 390.1 |
| 93 | 1,3-dihydro-1-(1,2,3,6-tetrahydro-4-pyridinyl)-2h-benzimidazole-2-one | 413.1 |
| 94 | 4-[4-chloro-3-(trifluoromethyl)phenyl]-4-piperidinol | 477.0 |
| 95 | 4-(2-keto-1-benzimidazolinyl)piperidine | 415.1 |
| 96 | 1-(4-chlorobenzhydryl)piperazine | 485.1 |
| 97 | (s)-(−)-1-methyl-2-(1-piperidino-methyl)pyrrolidine | 380.0 |
| 98 | 1-[5-(trifluoromethyl)pyrid-2-yl]-piperazine | 429.1 |
| 99 | 1-[3-(trifluoromethyl)pyrid-2-yl]piperazine | 429.1 |
| 100 | 4-cyano-4-phenylpiperidine HCl | 384.1 |
| 101 | 4-hydroxy-4-phenylpiperidine | 375.1 |
| 102 | 4-(1-pyrrolidinyl)piperidine | 352.2 |
| 103 | 4-acetyl-4-phenylpiperidine HCl | 401.1 |
| 104 | 4-(4-chlorophenyl)-1,2,3,6-tetrahydropyridine HCl | 392.1 |
| 105 | 1-piperazinepropanol | 342.1 |
| 106 | 1-(3-chlorophenyl)piperazine | 395.1 |

### 5-Methyl-2(S)-propyl-3(R)-[4-(3-trifluoromethyl-benzylamino)-piperidine-1-carbonyl]-hexanoic acid amide.

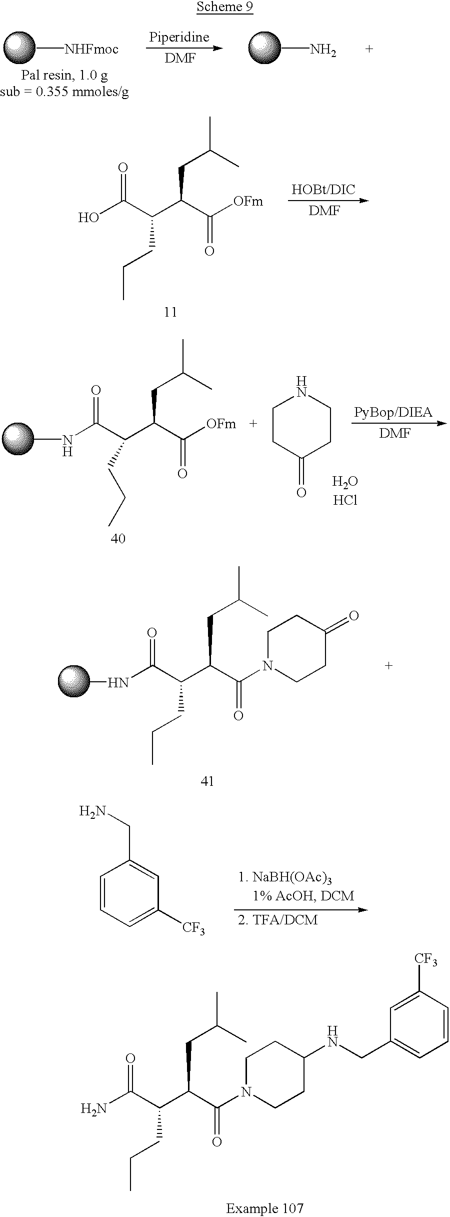

Fmoc-Pal resin (1.000 g, 0.355 mmol/g) was washed and deprotected with 50% Piperidine/DMF for 10 min. The resin was washed and suspended in DMF. Addition of 3 eq (1.065 mmoles, M.W.=394, 419.6 mg) of Succinic acid fluorenylmethyl ester (11) followed by 3 eq (1.065 mmoles, M.W.=153, 163 mg) of HOBt and 3 eq (1.065 mmoles, M.W.=126.2, d=0.806, 352 μL) of N,N-Diisopropylcarbodiimide and the reaction solution was allowed to shake overnight. A small sample was monitored by Ninhydrin test (negative). The resin was washed thoroughly with DMF, MeOH, CH₂Cl₂and DMF. About 100 mg (sub=0.033mmoles) of resin was taken and deprotected with 50% Piperidine/DMF for 10 min. The resin was washed thoroughly and suspended in DMF. Then 5 eq (0.165 mmoles, M.W.=153.61, 253 mg) of 4-Piperidone monohydrate.HCl was added followed by 5 eq (0.165 mmoles, M.W.=520.3, 86 mg) of PyBOP and 10 eq (0.33 mmoles, M.W.=129.25, d=0.742, 58 μL) of DIEA. Another 5 eq of DIEA was added to neutralize the HCL salt, and the reaction solution was allowed to shake overnight.

The resin was washed thoroughly with DMF, MeOH and CH₂Cl₂ and suspended in DCM. It was reductively alkylated with 5 eq (0.165 mmoles, M.W.=175.16, d=1.222, 24 μL) of 3-trifluoromethyl benzylamine followed by 5 eq (0.165 mmoles, M.W.=212, 35mg) of NaBH(OAc)₃ and 1% AcOH (v/v, 10 μL) and allowed to shake overnight. Next day, a small sample was checked with Chloranil test (positive). The resin was washed thoroughly with DMF, MeOH and CH₂Cl₂ and dried well under vacuum. The resin was treated with a mixture of TFA/CH₂Cl₂(9:1) for 2 h, filtered and concentrated in vacuum to give the crude compound. Purification by preparative LC/MS provided the title compound of example 107 as a powder(8 mg). MS (M+H)⁺=456.6.

### Examples 108-116

For each reagent listed in Table 2, the corresponding product was prepared according to the preparation of the compound of Example 107. The products of Examples 108-116 were verified by the presence of the desired compound in ESI MS (M+H)⁺.

TABLE 2

| | | | (M + H) + |
|---|---|---|---|
| Ex # | AMINE | Final Product | observed |
| 108 | 1-Naphthalene | 5-Methyl-3(R)-{4[(naphthalen-1- | 438.4 |
| | methylamine | ylmethyl)-amino]-piperidine-1- | |
| | | carbonyl}-2(S)-propyl-hexanoic | |
| | | acid amide | |
| 109 | 3,4-Methylene | 3(R)-[4-(Benzo[1,3]dioxol-5- | 418.4 |
| | dioxyaniline | ylamino)-piperidine-1-carbonyl]-5- | |
| | | methyl-2(S)-propyl-hexanoic acid | |
| | | amide. | |
| 110 | Aniline | 5-Methyl-3(R)-(4-phenylamino- | 374.4 |
| | | piperidine-1-carbonyl)-2(S)- | |
| | | propyl-hexanoic acid amide. | |
| 111 | m-Anisidine | 3(R)-[4-(3-Methoxy-phenylamino)- | 404.4 |
| | | piperidine-1-carbonyl]-5-methyl- | |
| | | 2(S)-propyl-hexanoic acid amide. | |
| 112 | Isopropylamine | 3(R)-(4-Isopropylamino-piperidine- | 340.4 |
| | | 1-carbonyl)-5-methyl-2(S)-propyl- | |
| | | hexanoic acid amide. | |
| 113 | 3-Methoxy-4- | 3(R)-[4-(3-Methoxy-4-methyl- | 418.4 |
| | methylaniline | phenylamino)-piperidine-1- | |
| | | carbonyl]-5-methyl-2(S)-propyl- | |
| | | hexanoic acid amide. | |
| 114 | Benzhydrylamine | 3(R)-[4-(Benzhydryl-amino)- | 464.4 |
| | | piperidine-1-carbonyl]-5-methyl- | |
| | | 2(S)-propyl-hexanoic acid amide | |
| 115 | 3-Fluoro-5- | 3(R)-[4-(3-Fluoro-5- | 474.4 |
| | (trifluoromethyl) | trifluoromethyl-benzylamino)- | |
| | benzylamine | piperidine-1-carbonyl]-5-methyl- | |
| | | 2(S)-propyl-hexanoic acid amide. | |
| 116 | 4- | 5-Methyl-2(S)-propyl-3(R)-[4-(4- | 442.4 |
| | Trifluoromethyl | trifluoro-methyl-phenylamino)- | |
| | aniline | piperidine-1-carbonyl]-hexanoic | |
| | | acid amide. | |

### Example 117

### N-[1-(3(S)-Carbamoyl-2(R)-isobutyl-hexanoyl)-piperidin-4-yl]-N-naphthalen-1-ylmethyl-benzamide.

Fmoc-Pal resin (1.000 g, 0.355 mmol/g) was washed and deprotected with 50% Piperidine/DMF for 10 min. The resin was washed and suspended in DMF. Addition of 3 eq (1.065 mmoles, M.W.=394, 419.6 mg) of Succinic acid fluorenylmethyl ester (11) followed by 3 eq (1.065 mmoles, M.W.=153, 163 mg) of HOBt and 3 eq (1.065 mmoles, M.W.=126.2, d=0.806, 352 μL) of N,N-Diisopropylcarbodiimide and the reaction solution was allowed to shake overnight. A small sample was monitored by Ninhydrin test (negative). The resin was washed thoroughly with DMF, MeOH, CH₂Cl₂ and DMF. About 100 mg (sub=0.033 mmoles) of resin was taken and deprotected with 50% Piperidine/DMF for 10 min. The resin was washed thoroughly and suspended in DMF. Then 5 eq (0.165 mmoles, M.W.=153.61, 253 mg) of 4-Piperidone monohydrate.HCl was added followed by 5 eq (0.165 mmoles, M.W.=520.3, 86 mg) of PyBOP and 10 eq (0.33 mmoles, M.W.=129.25, d=0.742, 58 μL) of DIEA. Another 5 eq of DIEA was added to neutralize the HCL salt, and the reaction solution was allowed to shake overnight.

The resin was washed thoroughly with DMF, MeOH and CH₂Cl₂ and suspended in DCM. It was reductively alkylated with 5 eq (0.165 mmoles, M.W.=157.16, 26 mg) of 1-naphthylmethylamine followed by 5 eq (0.165 mmoles, M.W.=212, 35 mg) of NaBH(OAc)₃ and 1% AcOH (v/v, 10 μL) and allowed to shake overnight. Next day, a small sample was checked with Chloranil test (positive).

The resin was washed thoroughly with DMF, MeOH and CH₂Cl₂ and dried well under vacuum. The resin was then suspended in DMF and acylated with 12 eq (0.075 mmoles, M.W.=129.25, d=0.742, 131 μL) of DIEA and 10 eq (0.625 mmoles, M.W.=140.57, d=1.211, 73 μL) of Benzoyl Chloride and allowed to shake overnight. The resin was then washed thoroughly with DMF, MeOH and CH₂Cl₂ and dried well under vacuum. The resin was cleaved with a mixture of TFA/ CH₂Cl₂(9:1) for 3 h, filtered and concentrated in vacuum to give the crude compound. Purification by preparative LC/MS provided the title compound of example 117 as a white powder MS (M+H)⁺=542.4.

### Examples 118-122

For each reagent listed in Table 3, the corresponding product was prepared according to the preparation of the compound of Example 117. The products of Examples 118-122 were verified by the presence of the desired compound in ESI MS (M+H)⁺.

TABLE 3

| | | | (M + H) + |
|---|---|---|---|
| Ex # | AMINE | Final Product | observed |
| 118 | 3,4-Methylene | N-Benzo[1,3]dioxol-5-yl-N-[1- | 522.3 |
| | dioxyaniline | (3(S)-carbamoyl-2(R)-isobutyl- | |
| | | hexanoyl)-piperidin-4-yl]- | |
| | | benzamide. | |
| 119 | Aniline | N-[1-(3(S)-Carbamoyl-2(R)- | 478.3 |
| | | isobutyl-hexanoyl)-piperidin-4-yl]- | |
| | | N-phenyl-benzamide. | |
| 120 | m-Anisidine | N-[1-(3(S)-Carbamoyl-2(R)- | 508.4 |
| | | isobutyl-hexanoyl)-piperidin-4-yl]- | |
| | | N-(3-methoxy-phenyl)-benzamide. | |
| 121 | Isopropylamine | N-[1-(3(S)-Carbamoyl-2(R)- | 444.4 |
| | | isobutyl-hexanoyl)-piperidin-4-yl]- | |
| | | N-isopropyl-benzamide. | |
| 122 | 3-Fluoro-5- | N-[1-(3(S)-Carbamoyl-2(R)- | 578.4 |
| | (trifluoromethyl) | isobutyl-hexanoyl)-piperidin-4-yl]- | |
| | benzylamine | N-(3-fluoro-5-trifluoromethyl- | |
| | | benzyl)-benzamide. | |

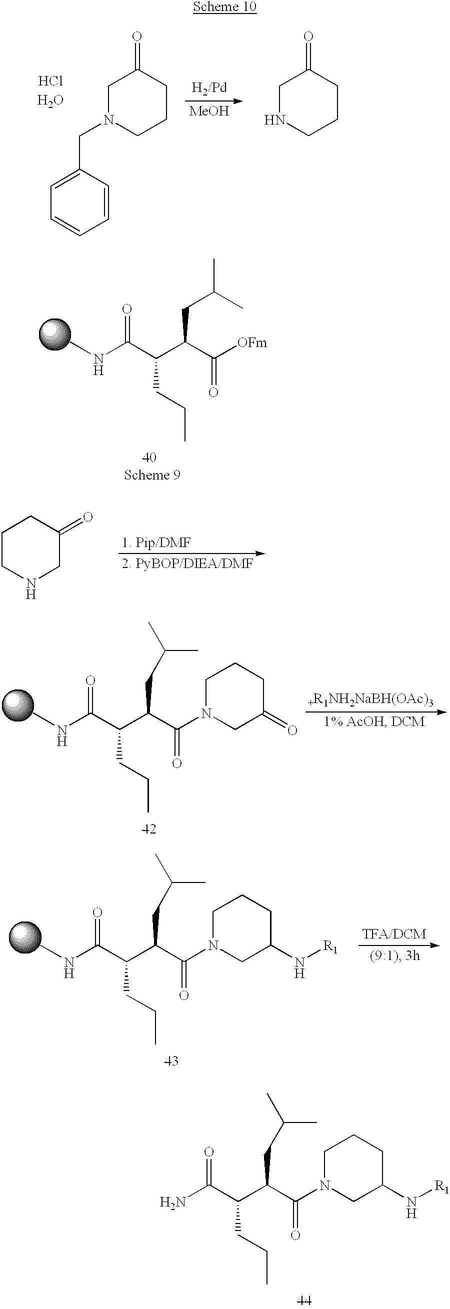

### Example 123

### 5-Methyl-3(R)-{3-[(naphthalen-1-ylmethyl)-amino]-piperidine-1-carbonyl}-2(S)-propyl-hexanoic acid amide

3-benzylpiperidine HC₁ hydrate 10 g, 41 mmol) was dissolved in 100 mL of methanol and placed in a Parr flask. A 0.5 g portion of 10% palladium on carbon was added and the reaction solution was shaken under 50 p.s.i. of dihydrogen for 16 h. The catalyst was removed by filtration and the solvent was removed in vacuo to provide the crude 3-piperidone which was used without further purification.

The compound of example 123 was then prepared according to the preparation of the compound of example 107 but using 3-piperidone, yielding 11 mg of the desired compound. MS (M+H)⁺=438.4.

### Examples 124-129

For each reagent listed in Table 4, the corresponding product was prepared according to the preparation of the compound of Example 123. The compounds of Examples 128 and 129 were prepared according to the preparation of the compound of Example 117, but using 3-piperidone. The products of Examples 124-129 were verified by the presence of the desired compound in ESI MS (M+H)⁺.

TABLE 4

| | | | (M + H) + |
|---|---|---|---|
| Ex # | AMINE | Product Structure | observed |
| 124 | 3-Methoxy-4- | 3(R)-[3-(3-Methoxy-4-methyl- | 418.4 |
| | methylaniline | phenylamino)-piperidine-1- | |
| | | carbonyl]-5-methyl-2(S)-propyl- | |
| | | hexanoic acid amide. | |
| 125 | Aniline | 5-Methyl-3(R)-(3-phenylamino- | 374.4 |
| | | piperidine-1-carbonyl)-2(S)- | |
| | | propyl-hexanoic acid amide. | |
| 126 | m-Anisidine | 3(R)-[3-(3-Methoxy-phenylamino)- | 404.4 |
| | | piperidine-1-carbonyl]-5-methyl- | |
| | | 2(S)-propyl-hexanoic acid amide. | |
| 127 | 3-Fluoro-5- | 3(R)-[3-(3-Fluoro-5- | 474.4 |
| | (trifluoromethyl) | trifluoromethyl-benzylamino)- | |
| | benzylamine | piperidine-1-carbonyl]-5-methyl- | |
| | | 2(S)-propyl-hexanoic acid amide. | |
| 128 | 1- | N-[1-(3(S)-Carbamoyl-2(R)- | 542.4 |
| | Naphthalene- | isobutyl-hexanoyl)-piperidin-3- | |
| | methylamine | yl]-N-naphthalen-1-ylmethyl- | |
| | | benzamide. | |
| 129 | 3-Fluoro-5- | N-[1-(3(S)-Carbamoyl-2(R)- | 578.4 |
| | (trifluoromethyl) | isobutyl-hexanoyl)-piperidin-3- | |
| | benzylamine | yl]-N-(3-fluoro-5- | |
| | | trifluoromethyl-benzyl)- | |
| | | benzamide. | |

### Example 130

### 3(R)-[4-Hydroxy-4-(4′-trifluoromethyl-biphenyl-4-yl)-piperidine-1-carbonyl]-5-methyl-2(S)-propyl-hexanoic acid amide

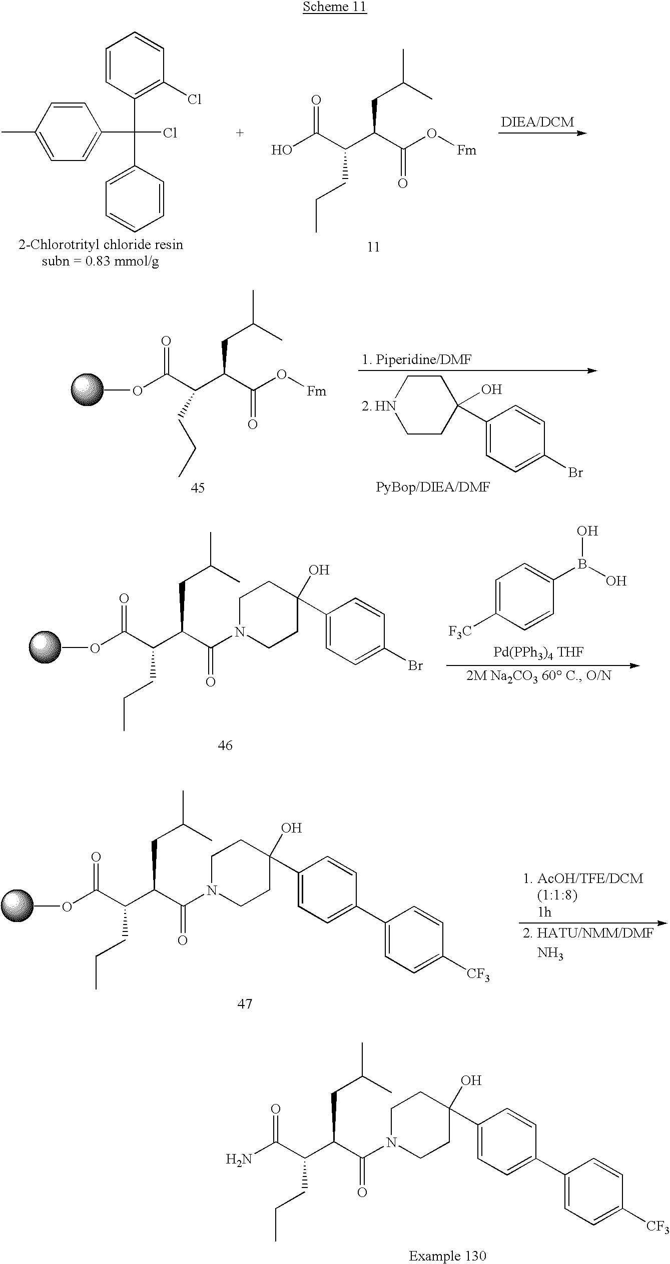

### Example 130(a)

2-Chlorotrityl chloride resin (Novabiochem, 0.250 g, 0.21 mmol) was washed and suspended in DCM. Then ˜2 eq (0.5 mmol, M.W.=394.5, 197 mg) of fluorenylmethyl protected succinic acid derivative was added and the resin was allowed to shake for 5 min. Then 2 eq (with respect to acid) (1.0 mmole, M.W.=129.25, d=0.742, 174 μL) of DIEA was added and the resin was allowed to shake overnight. The resin was washed thoroughly and the fluorenylmethyl group was deprotected with 50% Piperidine/DMF for 10 min and the resin was washed again.

### Example 130(b)

A 120 mg portion (0.1 mmol) of the resin from example 130(a) was suspended in DMF and then treated with 5 eq (0.5 mmol, M.W.=256.14, 128 mg) of 4-(4-Bromophenyl)-4-Piperidinol, 5 eq (o.5 mmol, M.W.=520.3, 260 mg) of PyBop and 10 eq (1.0 mmol, M.W.=129.25, d=0.742, 174 μL) of DIEA. The resin was allowed to shake overnight and then washed with DMF, dichloromethane, and methanol.

### Example 130(c)

The resin from example 130(b) (50 mg, 0.8 mmol/g, 0.040 μmol) was suspended in 1 mL of THF and 15 mg of tetrakis(triphenylphosphine)palladium (0), 70 mg (0.37 mmol) of 4-trifluoromethylphenyl boronic acid, and 200 μL of a 2 M sodium carbonate solution were added. The suspension was heated to 60° C. for 16 h, and the esin was isolated by filtration and washed with DMF, dichloromethane, and methanol.

Preparation of the Title Compound of Example 130

The resin from example 130(c) was suspended in 2 mL of a 1:1:8 solution of acetic acid, trifluoroethanol, and dichloromethane and the suspension was stirred for 1 h. Evaporation gave the crude acid which was dissolved in 1 mL of DMF and treated with HATU (4 mg, 0.01 mmol) and N-methylmorpholine (5 μL, 0.04 mmol). After 5 min ammonia was introduced by bubbling and the solution was allowed to stir for 16 h. The solution was then partitioned between ethyl acetate and water and the organic layer was isolated, dried and concentrated. Purification by RP-HPLC afforded 1.0 mg (10%) of the title compound of example 130. MS (M+H)⁺=519.4, (M+Na)⁺=541.4.

### Example 131

### 3(R)-(4-Biphenyl-4-yl-4-hydroxy-piperidine-1-carbonyl)-5-methyl-2(S)-propyl-hexanoic acid amide

The compound of Example 131 was prepared in a manner analogous to the preparation of the compound of Example 130, but using phenylboronic acid. Purification by RP-HPLC afforded 1 mg (10%) of the title compound of example 131. MS (M+H)⁺=451.4,

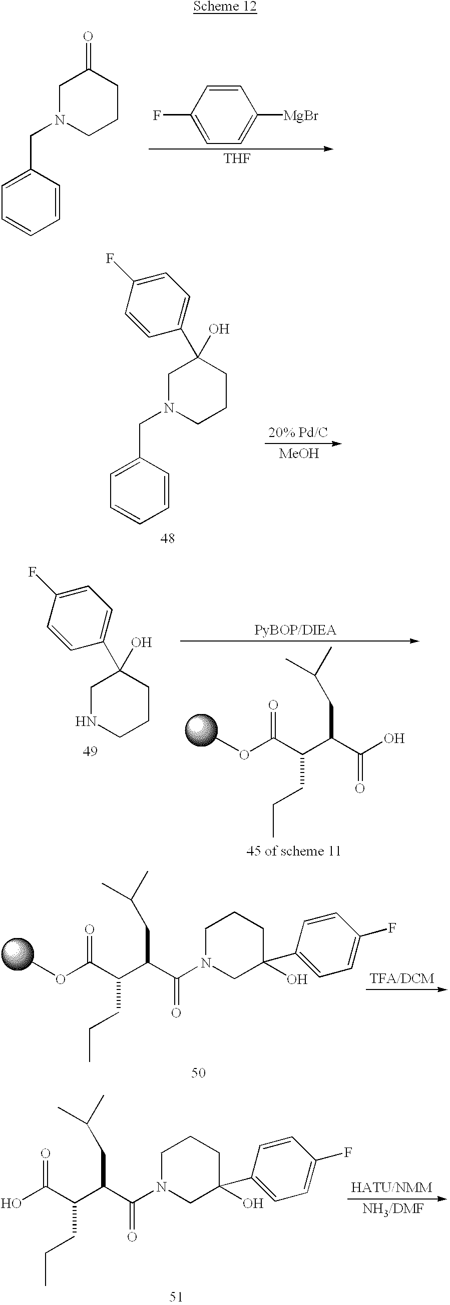

### Example 132

### 3(R)-[3-(4-Fluoro-phenyl)-3-hydroxy-piperidine-1-carbonyl]-5-methyl-2(S)-propyl-hexanoic acid amide

### Example 132(a)

To a solution of 2 g (10.6 mmol) of 3-piperidione in 50 mL of THF at 0° C. is added dropwise 10 mL of a 1M solition of 4-fluorophenylmagnesium bromide in THF. After 30 min, the reaction was quenched with 1N HCl and the THF was removed by rotary evaporation. The resultant aqueous layer was extracted twice with 50 mL of CH₂Cl₂ to provide 1.9 g (66%) of an oil which was used without further purification.

### Example 132(b)

The oil from above was dissolved in 25 mL of methanol and 380 mg of 20% paddadium on carbon was added. The reaction solution was placed under 50 p.s.i. of dihydrogen and shaken at rt for 16 h. The catalyst was then removed by filtration and the resulting piperidine was used without further purification.

### Example 132(c)

To a 0.2 g portion of resin from example 130(a) (0.16 mmol, 0.83 mmol/g) was added 0.83 mmol (162 mg) of the compound of example 135(b), 0.83 mmol (432 mg) of PyBop, and 1.66 mmol (289 μL) of DIEA. The suspension was stirred for 2 days and then the resin was washed thoroughly with DMF, DCM, and methanol. The resin was then suspended in 2 mL of a 1:1:8 solution of acetic acid, trifluoroethanol, and dichloromethane and the suspension was stirred for 2 h. Evaporation gave the crude acid (56 mg, 83%) which was used without further purification.

Preparation of the Title Compound of Example 132

The acid of example 132(c) (56 mg, 0.142 mmol) was dissolved in 2 mL of DMF and 70 mg (0.184 mmol) of HATU and 62 μL (0.57 mmol) of N-methylmorpholine was added. After 1 h ammonia gas was introduced by bubbling for 1 min and the reaction solution was allowed to stir for 16. The reaction solution was then partitioned between dichloromethane and water and the organic layer was separated, dried, and concentrated. Purification by RP-HPLC afforded 10 mg (18%) of the title compound of example 132 as a white powder. MS (M+H)⁺=393.5, (M+Na)⁺=415.4.

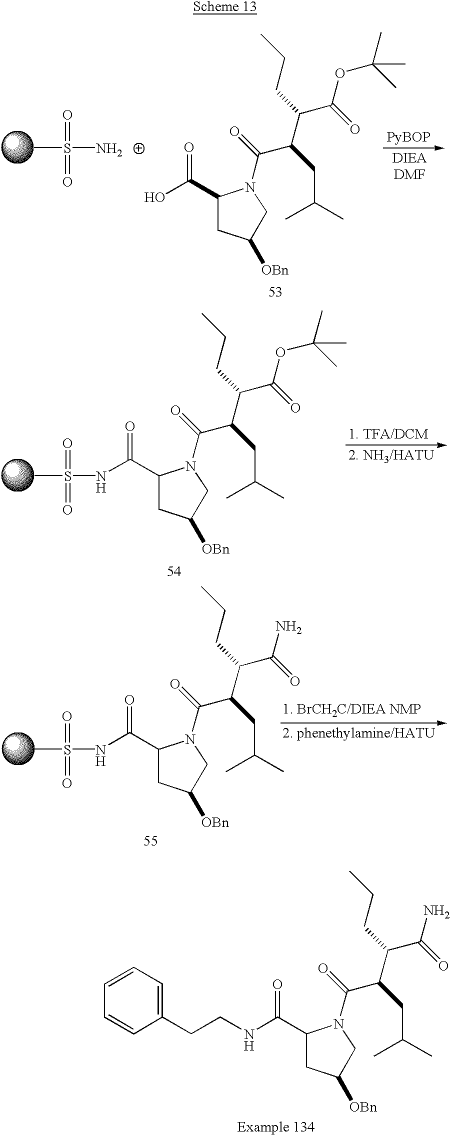

### Example 134

### 4(S)-Benzyloxy-1-(3(S)-carbamoyl-2(R)-isobutyl-hexanoyl)-pyrrolidine-2(S)-carboxylic acid phenethyl-amide

### Example 134(a)

7.3 g of succinate 10 of scheme 2 was dissolved in 70 mL of DMF and activated with 13.3 g of HATU and 14.73 mL of N-methylmorpholine. After stirring at rt for 30 min 7.4 g of 4(S)-benzylhydroxyproline methyl ester hydrochloride was added and the reaction solution was stirred at rt for 2 h. The reaction solution was diluted with 100 mL of water and the resulting solution was extracted 3× with ethyl acetate. The combined organic layers were dried and concentrated and ther residue was purified by chromatography eluting with 10-25% ethyl acteate in hexanes to provide 8.4 g (66%) of the desired amide. MS (M+H)⁺=490.4

### Example 134(b)

The methyl ester from example 134(a) (8.4 g, 17.1 mmol) in 30 mL of dioxane was cooled to 0° C. and 20 mL of 1 N NaOH was added. The solution was stirred for 2 h and additional portions of dioxane (15 mL) and NaOH (20 mL) were added, followed by stiring for another 2 h. The reaction solution was then acidified to pH 3 with citric acid and then extracted 3× with ethyl acetate. The combined organic layers were dried and concentrated to provide the crude acid which required no further purification. MS (M+H)⁺=476.3

### Example 134(c)

Alkanesufonamide safety catch resin (Novabiochem, 4.5 g, 0.8 mmol/g, 3.6 mmol) was washed well and then suspended in 50 mL of DMF. The acid from example 134(b) (5.133 g, 10.8 mmol), PyBop (5.62 g, 10.8 mmol) and DIEA (5.65 mL, 32.4 mmol) were added and the suspension was shaken for 16 h. The resin was then rinsed thoroughly with DMF, dichloromethane, and methanol and dried.

### Example 134(d)

A 25 mg portion of the resin from example 134(c) (0.02 mmol) was suspended in a 1:1 solution of dichloromethane and tricluoroacetic acid (0.5 mL) and allowed to shake for 2 h at rt. The resin was then washed thoroughly, and resuspended in 0.5 mL of DMF.and treated with HATU (38 mg, 0.1 mmol) and 150 mL of a saturated solution of ammonia in THF. The reaction suspension was allowed to stir at rt for 1.5 h and then the resin was washed thoroughly.

Preparation of the Title Compound of Example 134

The resin from example 138(d) was suspended in 0.5 mL of NMP and activated with 0.1 mmol of DIEA (18 μL) and 0.25 mmol (30 μL) of bromoacetonitrile at rt for 16 h. The resin was then washed thoroughly and suspended in 300 uL of THF to which 0.008 mmol of phenethylamine (40 uL of a 0.2M solution) was added. The reaction solution was stirred at rt for 2 days and then concentrated to provide 2.6 mg of the title compound of example 134 (63%). MS (M+H)⁺=522.3, MS ESI⁻, (M−H)⁻=520.2.

Tables 5a-5g below provide representative Examples of the compounds of Formula (I) of the present invention.

TABLE 5a

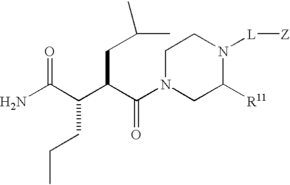

| | | | | Molecular Weight |
|---|---|---|---|---|
| Ex # | L | Z | R¹¹ | of Product |
| 1 | —CH₂— | 4-benzo[1,3]dioxol-5-yl | H | 417.54 |
| 2 | — | H | H | 283.407 |
| 3 | — | phenyl | H | 359.505 |
| 4 | — | 2-MeO-phenyl | H | 389.53 |
| 5 | — | 3-CF₃-phenyl | H | 427.502 |
| 6 | — | 4-F-phenyl | H | 377.495 |
| 7 | — | 4-NO₂-phenyl | H | 404.502 |
| 8 | —CH₂— | H | H | 297.434 |
| 9 | —CH₂— | phenyl | H | 373.531 |
| 10 | —CH₂CH₂O— | H | H | 327.46 |
| 11 | — | 2-pyridyl | H | 360.493 |
| 12 | — | 2-Cl-phenyl | H | 394 |
| 13 | — | phenyl | Me | 373.531 |
| 14 | — | 4-MeO-phenyl | Me | 403.557 |
| 15 | — | 4-Me-phenyl | H | 373.5 |
| 16 | — | 3-MeO-phenyl | H | 389.5 |
| 18 | — | 3-Me-phenyl | Me | 387.558 |
| 20 | —CH₂CH₂— | H | H | 311.461 |
| 21 | —CH₂CH═CH₂— | phenyl | H | 399.569 |
| 22 | —(CH₂)₂—O—(CH₂)₂— | H | H | 371.512 |
| 23 | —(CH₂)₂—NH—CH₂— | 2-pyridyl | H | 417.588 |
| 24 | — | 2-Me-5-Cl-phenyl | H | 407.976 |
| 65 | — | 4-Cl-phenyl | H | 394 |
| 66 | — | 2-EtO-phenyl | H | 403.6 |
| 67 | — | 4-F-phenyl | H | 377.5 |
| 68 | — | 2,4-diMe-phenyl | H | 387.561 |
| 69 | — | 4-Cl-phenyl | Me | 407.979 |
| 70 | — | 3,4-diCl-phenyl | H | 428.397 |
| 71 | — | 3,4-diMe-phenyl | H | 387.561 |
| 72 | — | 2,6-diMe-phenyl | H | 387.561 |
| 73 | — | 3-Cl-phenyl | H | 394 |
| 74 | — | 2-F-phenyl | H | 377.497 |
| 75 | — | 2-Cl-phenyl | H | 393.952 |
| 76 | — | 2-NO₂-phenyl | H | 404.504 |
| 77 | — | 2-Me-phenyl | H | 373.534 |
| 78 | — | 2-Et-phenyl | H | 387.561 |
| 79 | — | 3-Me-phenyl | H | 373.534 |
| 80 | — | 3-CF₃-4-Cl-phenyl | H | 461.95 |
| 81 | — | 4-Me-phenyl | H | 373.534 |
| 82 | — | 2-pyrimidyl | H | 361.482 |
| 83 | — | 2,3-diMe-phenyl | H | 387.561 |
| 84 | — | 4-pyridyl | H | 360.494 |
| 85 | — | 3,5-diCl-phenyl | H | 428.397 |
| 86 | — | 4-CF₃-phenyl | H | 427.505 |
| 87 | — | 2-pyrazinyl | H | 361.482 |
| 88 | — | 2-CN-phenyl | H | 384.516 |
| 89 | — | 2,4-diMeO-phenyl | H | 419.559 |
| 90 | — | 4-benzo[1,3]dioxol-5-yl | H | 403.5 |
| 91 | — | 4-Me-phenyl | Me | 387.561 |
| 92 | — | 3-MeO-phenyl | H | 389.5 |
| 96 | — | 4-chlorobenzhydryl | H | 485.1 |
| 98 | — | 5-CF₃-pyrid-2-yl | H | 428.492 |
| 99 | — | 3-CF₃-pyrid-2-yl | H | 428.492 |
| 105 | —(CH₂)₃—O— | H | H | 341.486 |
| 106 | — | 3-Cl-phenyl | H | 393.95 |

TABLE 5a″

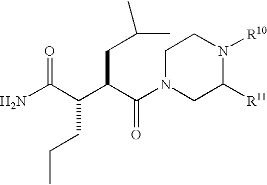

| | | | Molecular Weight |
|---|---|---|---|
| Ex # | R¹⁰ | R¹¹ | of Product |
| 17 | —CH₂C(═O)OEt | H | 369.496 |
| 19 | —C(═O)Me | H | 325.444 |
| 96 | 4-Cl-benzhydryl | H | 484.077 |

TABLE 5b

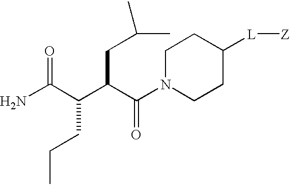

| Ex # | L | Z | Mol Wt |
|---|---|---|---|
| 26 | — | 2-keto-1-benzimidazolinyl | 414.54 |
| 36 | O | H | 298.418 |
| 38 | —CH₂— | H | 296.446 |
| 39 | —CH₂— | phenyl | 372.543 |
| 40 | —CH₂—NH— | H | 311.461 |
| 41 | —(CH₂)₂—O— | H | 326.472 |
| 42 | — | N-piperidyl | 365.552 |
| 44 | —CH₂CH₂— | 4-piperidyl | 393.6 |
| 49 | —(CH₂)₃— | phenyl | 400.597 |
| 50 | —(CH₂)₃— | H | 324.499 |
| 58 | —CH₂—NH—(CH₂)₂— | NH₂ | 354.529 |
| 102 | — | 1-pyrrolidinyl | 351.527 |
| 107 | —NH—CH₂— | 3-CF₃-phenyl | 456.6 |
| 108 | —NH—CH₂— | naphthalen-1-yl | 438.4 |
| 109 | —NH— | 3,4-(methylendioxy)-phenyl | 418.4 |
| 110 | —NH— | phenyl | 374.4 |
| 111 | —NH— | 3-MeO-phenyl | 404.4 |
| 112 | —NH— | i-propyl | 340.4 |
| 113 | —NH— | 3-MeO-4-Me-phenyl | 418.4 |
| 114 | —NH— | benzhydryl | 464.4 |
| 115 | —NH—CH₂— | 3-CF₃-5-F-phenyl | 474.4 |
| 116 | —NH— | 4-CF₃-phenyl | 442.4 |
| 117 | —N(benzoyl)-CH₂— | naphthalen-1-yl | 542.4 |
| 118 | —N(benzoyl)- | 3,4-(methylendioxy)-phenyl | 522.33 |
| 119 | —N(benzoyl)- | phenyl | 478.3 |
| 120 | —N(benzoyl)- | 3-MeO-phenyl | 508.4 |
| 121 | —N(benzoyl)- | i-propyl | 444.4 |
| 122 | —N(benzoyl)-CH₂— | 3-CF₃-5-F-phenyl | 578.4 |

TABLE 5c

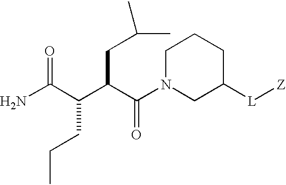

| Ex # | L | Z | Mol Wt |
|---|---|---|---|
| 123 | —NH—CH₂— | naphthalen-1-yl | 438.4 |
| 124 | —NH— | 3-MeO-4-Me-phenyl | 418.4 |
| 125 | —NH— | phenyl | 374.4 |
| 126 | —NH— | 3-MeO-phenyl | 404.4 |
| 127 | —NH—CH₂— | 3-CF₃-5-F-phenyl | 474.4 |
| 128 | —N(benzoyl)-CH₂— | naphthalen-1-yl | 542.4 |
| 129 | —N(benzoyl)-CH₂— | 3-CF₃-5-F-phenyl | 578.4 |

TABLE 5d

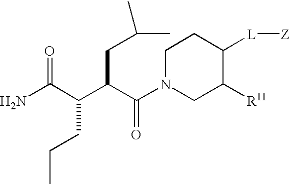

| Ex # | L | Z | R¹¹ | Mol Wt |
|---|---|---|---|---|
| 30 | — | H | —C(═O)NH₂ | 325.444 |
| 31 | — | H | —C(═O)OH | 326.428 |
| 32 | — | H | —C(═O)OEt | 354.482 |
| 33 | — | H | —C(═O)N(Et)₂ | 381.551 |
| 35 | — | H | —CH₂OH | 312.445 |
| 45 | — | H | —OH | 298.418 |
| 62 | — | 3-CF₃-phenyl | —OH | 442.5 |
| 64 | — | H | —OH | 298.4 |
| 133 | — | 3-CF₃-phenyl | —OH | |

TABLE 5e

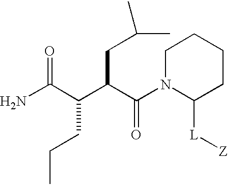

| | | | Molecular |
|---|---|---|---|
| | | | Weight |
| Ex # | L | Z | of Product |
| 27 | — | methyl | 296.446 |
| 28 | — | —C(═O)OEt | 354.482 |
| 29 | — | —CH₂OH | 312.445 |
| 46 | —CH₂CH₂NH— | 3,5-bis-CF₃-phenyl | 537.579 |
| 47 | —CH₂CH₂NH— | 4-iPr-phenyl | 443.665 |
| 55 | — | —C(═O)OH | 326.428 |
| 56 | —CH₂— | N-piperidino | 379.579 |
| 97 | —CH₂— | 1-Me-pyrrolidin-2-yl | 379.581 |

TABLE 5f

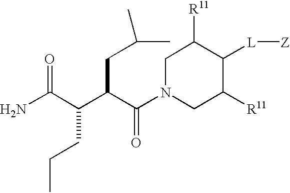

| | | | | Molecular |
|---|---|---|---|---|
| | | | | Weight |
| Ex # | L | Z | R¹¹ | of Product |
| 34 | — | H | Me | 310.472 |

TABLE 5g

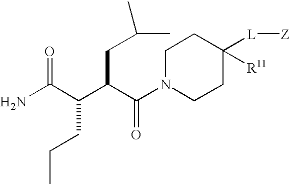

| Ex # | L | Z | R¹¹ | Mol Wt |
|---|---|---|---|---|
| 37 | — | H | —C(═O)OEt | 354.482 |
| 48 | — | H | —N(Me)₂ | 325.487 |
| 51 | — | phenyl | —C(═O)Et | 414.6 |
| 52 | —N(Me)— | Me | —C(═O)NH₂ | 368.5 |
| 53 | — | H | 1-pyrrolidinyl | 351.525 |
| 54 | — | H | —C(═O)NH₂ | 325.444 |
| 57 | —NH— | phenyl | —C(═O)NH₂ | 416.556 |
| 59 | —NH— | cyclohexyl | —C(═O)NH₂ | 422.604 |
| 60 | —NH— | Et | —C(═O)NH₂ | 368.512 |
| 61 | — | phenyl | Me | 372.543 |
| 94 | — | 4-Cl-3-CF₃-phenyl | —OH | 476.962 |
| 100 | — | phenyl | —CN | 383.5 |
| 101 | — | phenyl | —OH | 374.518 |
| 103 | — | phenyl | —C(═O)Me | 400.6 |
| 104 | — | 4-Cl-phenyl | —OH | 391 |
| 130 | — | 4-(4-CF₃-phenyl)-phenyl | —OH | 519.4 |
| 131 | — | 4-(phenyl)-phenyl | —OH | 451.4 |
| 132 | — | 4-F-phenyl | —OH | 393.5 |

### UTILITY

Aβ production has been implicated in the pathology of Alzheimer's Disease (Aβ). The compounds of the present invention have utility for the prevention and treatment of AD by inhibiting Aβ production. Methods of treatment target formation of Aβ production through the enzymes involved in the proteolytic processing of β-amyloid precursor protein. Compounds that inhibit β or γ secretase activity, either directly or indirectly, control the production of Aβ. Such inhibition of β or γ secretases reduces production of Aβ, and is expected to reduce or prevent the neurological disorders associated with Aβ protein, such as Alzheimer's Disease.

Cellular screening methods for inhibitors of Aβ production, testing methods for the in vivo suppression of Aβ production, and assays for the detection of secretase activity are known in the art and have been disclosed in numerous publications, including J.Med.Chem. 1999, 42, 3889-3898, PCT publication number WO 98/22493, EPO publication number 0652009, U.S. Pat. Nos. 5,703,129 and 5,593,846; all hereby incorporated by reference.

The compounds of the present invention have utility for the prevention and treatment of disorders involving Aβ production, such as cerebrovascular disorders.

Compounds of Formula (I) are expected to possess γ-secretase inhibitory activity. The γ-secretase inhibitory activity of the compounds of the present invention is demonstrated using assays for such activity, for Example, using the assay described below. Compounds of the present invention have been shown to inhibit the activity of γ-secretase, as determined by the Aβ immunoprecipitation assay.

Compounds provided by this invention should also be useful as standards and reagents in determining the ability of a potential pharmaceutical to inhibit Aβ production. These would be provided in commercial kits comprising a compound of this invention.

As used herein “μg” denotes microgram, “mg” denotes milligram, “g” denotes gram, “μL” denotes microliter, “mL” denotes milliliter, “L” denotes liter, “mM” denotes nanomolar, “μM” denotes micromolar, “mM” denotes millimolar, “M” denotes molar, “nm” denotes nanometer, “SDS” denotes sodium dodecyl sulfate, and “DMSO” denotes dimethyl sulfoxide, and “EDTA” denotes ethylenediaminetetraacetato.

A compound is considered to be active if it has an IC₅₀ or Kᵢ value of less than about 100 μM for the inhibition of Aβ production. Preferrably the IC₅₀ or Kᵢ value is less than about 10 μM; more preferrably the IC₅₀ or Kᵢ value is less than about 0.1 μM. The present invention has been shown to inhibit Aβ protein production with an IC₅₀ or Kᵢ value of less than 100 μM.

β Amyloid Precursor Protein Accumulation Assay (βAPPA Assay)

An assay to evaluate the accumulation of Aβ protein was developed to detect potential inhibitors of secretases. The assay uses the N 9 cell line, characterized for expression of exogenous APP by immunoblotting and immunoprecipitation.

The effect of test compounds on the accumulation of Aβ in the conditioned medium is tested by immunoprecipitation. N 9 cells are grown to confluency in 6-well plates and washed twice with 1×Hank's buffered salt solution. The cells are starved in methionine/cysteine deficient media for 30 min., followed by replacement with fresh deficient media containing 150 uCi Tran35S-LABEL™ (ICN). Test compounds dissolved in DMSO (final concentration 1%) are added, over a range of 1 picomolar to 100 micromolar, together with the addition of the fresh media containing Tran35S-LABEL™. The cells are incubated for 4 h at 37° C. in a tissue culture incubator.

At the end of the incubation period, the conditioned medium is harvested and pre-cleared by the addition of 5 μl normal mouse serum and 50 ul of protein A Sepharose (Pharmacia), mixed by end-over-end rotation for 30 minutes at 4° C., followed by a brief centrifugation in a microfuge. The supernatant is then harvested and transferred to fresh tubes containing 5 ug of a monoclonal antibody (examples of antibodies include but are not limited by, clone 1101.1, directed against an internal peptide sequence in Aβ; or 6E10 from Senetek; or 4G8 from Senetek; additionally polyclonals from rabbit antihuman Aβ from Boehringer Mannheim) and 50 ul protein A Sepharose. After incubation overnight at 4° C., the samples are washed three times with high salt washing buffer (50 mM Tris, pH 7.5, 500 mM NaCl, 5 mM EDTA, 0.5% Nonidet P-40), three times with low salt wash buffer (50 mM Tris, pH 7.5, 150 mM NaCl, 5 mM EDTA, 0.5% Nonidet P-40), and three times with 10 mM Tris, pH 7.5. The pellet after the last wash is resuspended in SDS sample buffer (Laemmli U. K. Cleavage of structural proteins during the assembly of the head of bacteriphage T4. Nature 227, 680-5, 1970.) and boiled for 3 minutes. The supernatant is then fractionated on either 10-20% Tris/Tricine SDS gels or on 16.5% Tris/Tricine SDS gels. The gels are dried and exposed to X-ray film or analyzed by phosphorimaging. The resulting image is analyzed for the presence of Aβ polypeptides. The steady-state level of Aβ in the presence of a test compound is compared to wells treated with DMSO (1%) alone. A typical test compound in this assay blocks Aβ accumulation in the conditioned medium, and is considered active with an IC₅₀ less than 100 μM.

C-Terminus β-Amyloid Precursor Protein Accumulation Assay (CTF Assay)

The effect of test compounds on the accumulation of C-terminal fragments is determined by immunoprecipitation of APP and fragments thereof from cell lysates. N 9 cells are metabolically labeled, as above, with media containing Tran35S-LABEL™, in the presence or absence of test compounds. At the end of the incubation period, the conditioned medium are harvested and cells lysed in RIPA buffer (10 mM Tris, pH 8.0 containing 1% Triton X-100, 1% deoxycholate, 0.1% SDS, 150 mM NaCl, 0.125% NaN₃). Again, lysates are precleared with 5 ul normal rabbit serum/50 ul protein A Sepharose, followed by the addition of BC-1 antiserum (15 μl;) and 50 μl protein A Sepharose for 16 hours at 4° C. The immunoprecipitates are washed as above, bound proteins eluted by boiling in SDS sample buffer and fractionated by Tris/Tricine SDS-PAGE. After exposure to X-ray film or phosphorimager, the resulting images are analyzed for the presence of C-terminal APP fragments. The steady-state level of C-terminal APP fragments is compared to wells treated with DMSO (1%) alone. A typical test compound in this assay stimulates C-terminal fragment accumulation in the cell lysates, and is considered active with an IC₅₀ less than 100 μM.

Accumulation-release Assay

This immunoprecipitation assay is specific for g secretase activity (i.e., proteolytic activity required to generate the C-terminal end of Aβ either by direct cleavage or generating a C-terminal extended species which is subsequently further proteolyzed). N 9 cells are pulse labeled with media containing Tran35S-LABEL™ in the presence of a reported g secretase inhibitor (MDL 28170; Higaki J, Quon D, Zhong Z, Cordell B. Inhibition of beta-amyloid formation identifies proteolytic precursors and subcellular site of catabolism. Neuron 14, 651-659, 1995) for 1 h, followed by washing to remove ³⁵S radiolabel and MDL 28170. The media is replaced and test compounds are added over a dose range (for example 0.1 nM to 100 μM). The cells are chased for increasing periods of times and Aβ is isolated from the conditioned medium and C-terminal fragments from cell lysates (see accumulation assay above). The activity of test compounds are characterized by whether a stabilization of C-terminal fragments is observed and whether Aβ is generated from these accumulated precursor. A typical test compound in this assay prevents the generation of Aβ out of accumulated C-terminal fragments and is considered active with an IC₅₀ less than 100 μM.

### Dosage and Formulation

The compounds determined from the present invention can be administered orally using any pharmaceutically acceptable dosage form known in the art for such administration. The active ingredient can be supplied in solid dosage forms such as dry powders, granules, tablets or capsules, or in liquid dosage forms, such as syrups or aqueous suspensions. The active ingredient can be administered alone, but is generally administered with a pharmaceutical carrier. A valuable treatise with respect to pharmaceutical dosage forms is Remington's Pharmaceutical Sciences, Mack Publishing.

The compounds determined from the present invention can be administered in such oral dosage forms as tablets, capsules (each of which includes sustained release or timed release formulations), pills, powders, granules, elixirs, tinctures, suspensions, syrups, and emulsions. Likewise, they may also be administered in intravenous (bolus or infusion), intraperitoneal, subcutaneous, or intramuscular form, all using dosage forms well known to those of ordinary skill in the pharmaceutical arts. An effective but non-toxic amount of the compound desired can be employed to prevent or treat neurological disorders related to β-amyloid production or accumulation, such as Alzheimer's disease and Down's Syndrome.

The compounds of this invention can be administered by any means that produces contact of the active agent with the agent's site of action in the body of a host, such as a human or a mammal. They can be administered by any conventional means available for use in conjunction with pharmaceuticals, either as individual therapeutic agents or in a combination of therapeutic agents. They can be administered alone, but generally administered with a pharmaceutical carrier selected on the basis of the chosen route of administration and standard pharmaceutical practice.

The dosage regimen for the compounds determined from the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient,and the effect desired. An ordinarily skilled physician or veterinarian can readily determine and prescribe the effective amount of the drug required to prevent, counter, or arrest the progress of the condition.

Advantageously, compounds determined from the present invention may be administered in a single daily dose, or the total daily dosage may be administered in divided doses of two, three, or four times daily.

The compounds identified using the present invention can be administered in intranasal form via topical use of suitable intranasal vehicles, or via transdermal routes, using those forms of transdermal skin patches wall known to those of ordinary skill in that art. To be administered in the form of a transdermal delivery system, the dosage administration will, of course, be continuous rather than intermittent throughout the dosage regimen.

In the methods of the present invention, the compounds herein described in detail can form the active ingredient, and are typically administered in admixture with suitable pharmaceutical diluents, excipients, or carriers (collectively referred to herein as carrier materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl callulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or β-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds determined from the present invention can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds determined from the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington's Pharmaceutical Sciences, Mack Publishing Company, a standard reference text in this field.

Table 6 demonstrates representative substituents on the left end, or succinate end, of the compound of Formula (I), showing compounds envisaged within the scope of the present invention. Each of the fragments a through bt is attached to A, below.

TABLE 6

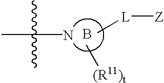

A

| | |
|---|---|
| 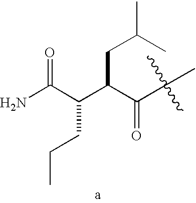 | 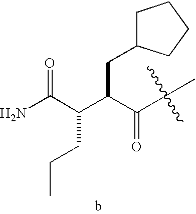 |
| 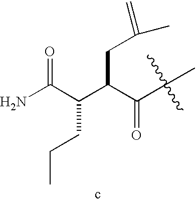 | 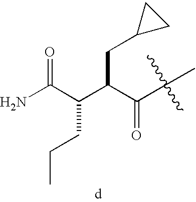 |
| 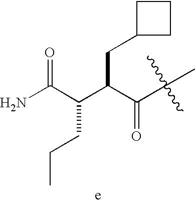 | 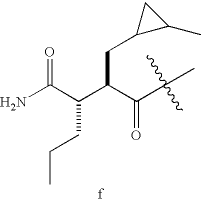 |
| 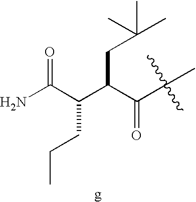 | 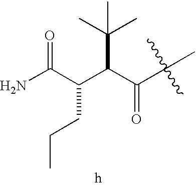 |
| 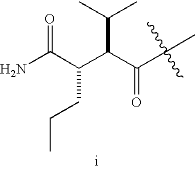 | 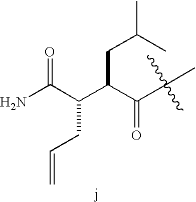 |
| 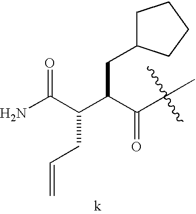 | 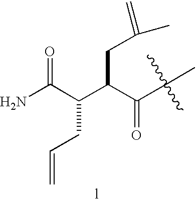 |
| 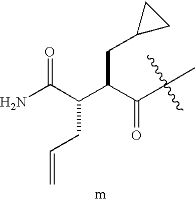 | 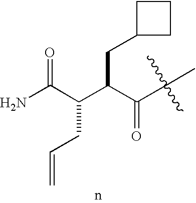 |
| 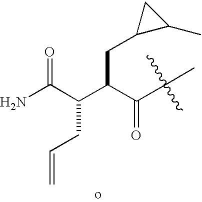 | 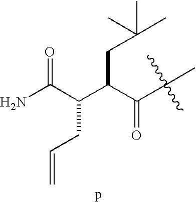 |
| 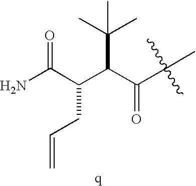 | 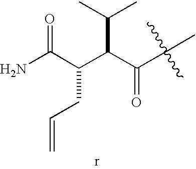 |
| 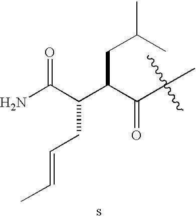 | 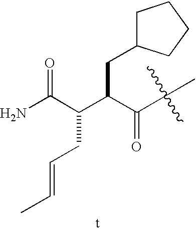 |
| 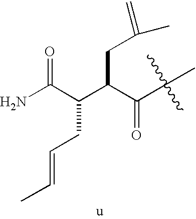 | 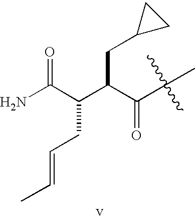 |
| 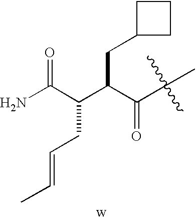 | 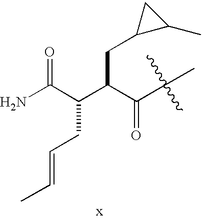 |
| 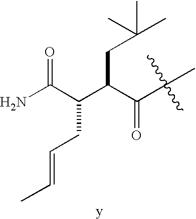 | 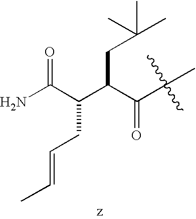 |
| 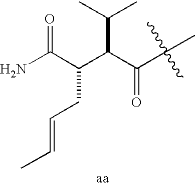 | 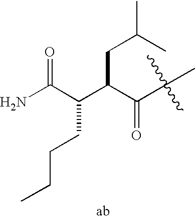 |
| 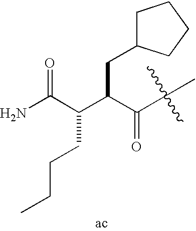 | 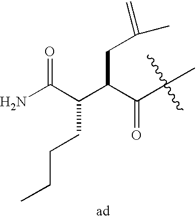 |
| 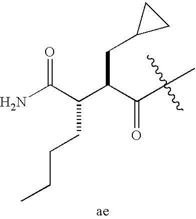 | 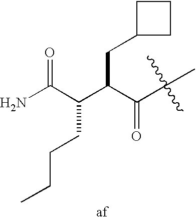 |
| 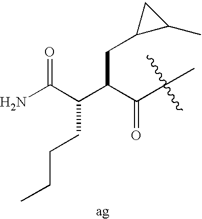 | 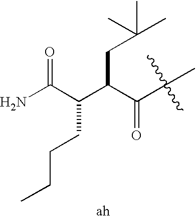 |
| 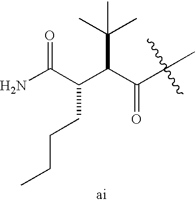 | 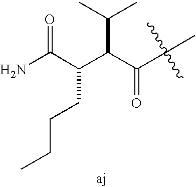 |
| 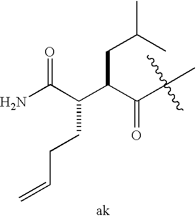 | 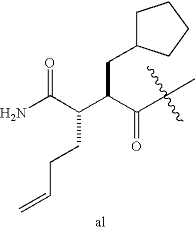 |
| 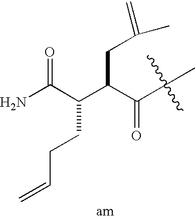 | 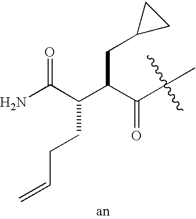 |
| 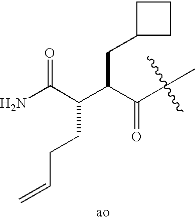 | 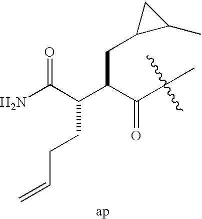 |
| 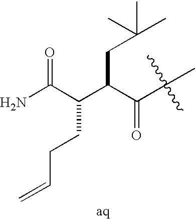 | 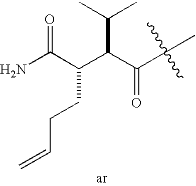 |
| 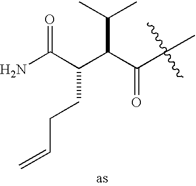 | 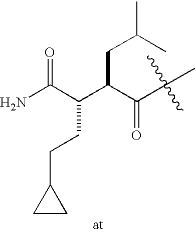 |
| 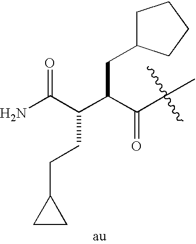 | 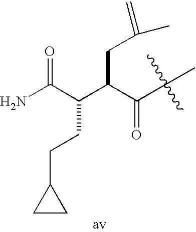 |
| 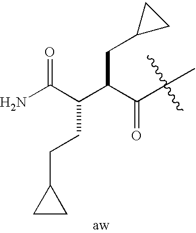 | 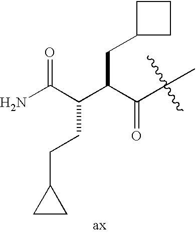 |
| 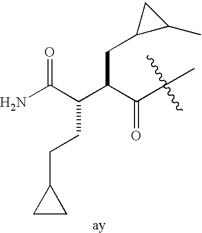 | 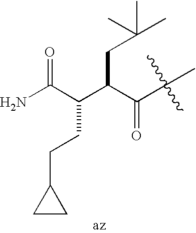 |
| 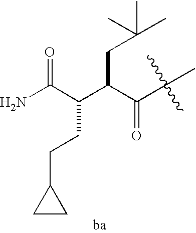 | 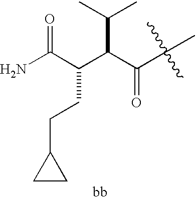 |
| 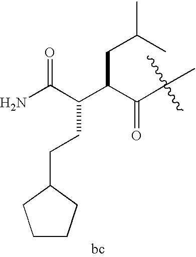 | 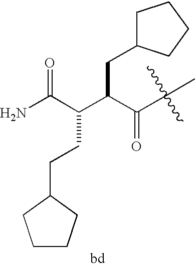 |
| 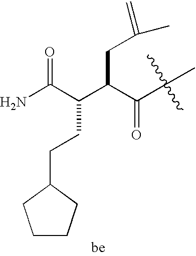 | 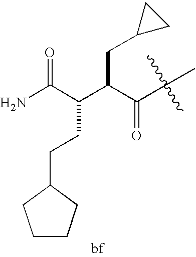 |
| 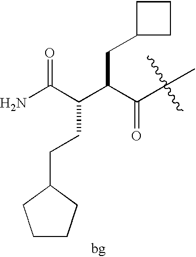 | 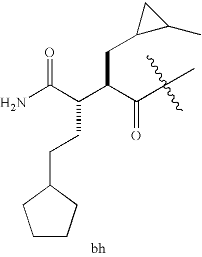 |
| 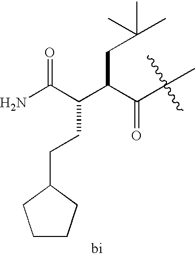 | 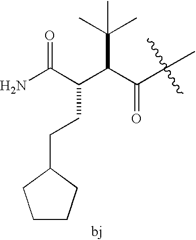 |
| 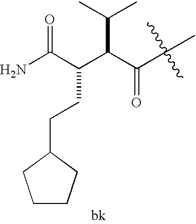 | 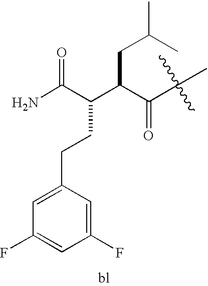 |
| 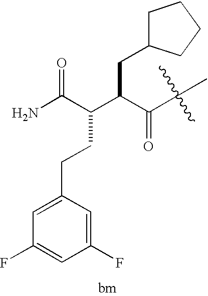 | 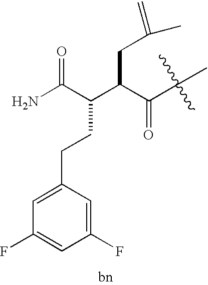 |
| 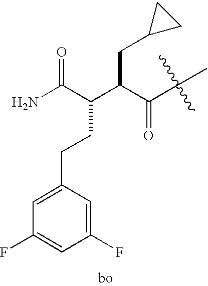 | 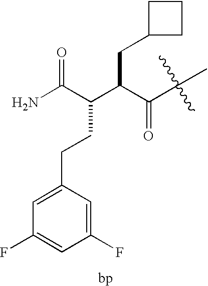 |
| 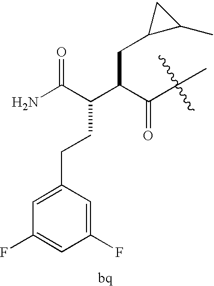 | 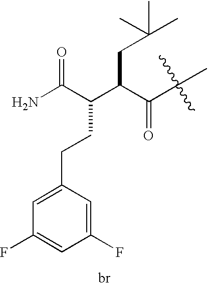 |
| 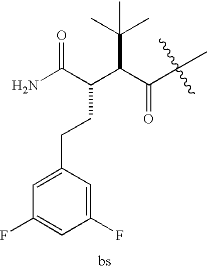 | 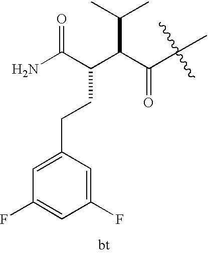 |

What is claimed is:

1. A compound of Formula (I): 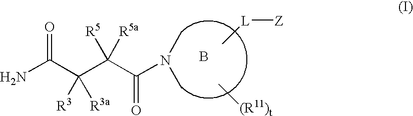
or a pharmaceutically acceptable salt or prodrug thereof, wherein:
R³ is —(CR⁷R^{7a})ₙ—R⁴,
—(CR⁷R^{7a})ₙ—S—(CR⁷R^{7a})ₘ—R⁴,
—(CR⁷R^{7a})ₙ—O—(CR⁷R^{7a})ₘ—R⁴,
—(CR⁷R^{7a})ₙ—N(R^{7b})—(CR⁷R^{7a})ₘ—R⁴,
—(CR⁷R^{7a})ₙ—S(═O)—(CR⁷R^{7a})ₘ—R⁴,
—(CR⁷R^{7a})ₙ—S(═O)₂—(CR⁷R^{7a})ₘ—R⁴,
—(CR⁷R^{7a})ₙ—C(═O)—(CR⁷R^{7a})ₘ—R⁴,
—(CR⁷R^{7a})ₙ—N(R^{7b})C(═O) (CR⁷R^{7a})ₘ—R⁴,
—(CR⁷R^{7a})ₙ—C(═O)N(R^{7b})—(CR⁷R^{7a})ₘ—R⁴,
—(CR⁷R^{7a})ₙ—N(R^{7b})S(═O)₂—(CR⁷R^{7a})ₘ—R⁴, or
—(CR⁷R^{7a})ₙ—S(═O)₂N(R^{7b})—(CR⁷R^{7a})ₘ—R⁴;
provided R³ is not hydrogen when R⁵ is hydrogen;
n is 0, 1, 2, or 3;
m is 0, 1, 2, or 3;
R^{3a} is H, OH, C₁-C₄ alkyl, C₁-C₄ alkoxy, C₂-C₄ alkenyl, or C₂-C₄ alkenyloxy;
alternatively, R³ and R^{3a}, and the carbon to which they are attached, may be combined to form a 3-8 membered cycloalkyl moiety substituted with 0-2 R^{4b}; provided that R⁵ and R^{5a} are not combined to form a 3-8 membered cycloalkyl moiety;
R⁴ is H, OH, OR^{14a},
C₁-C₆ alkyl substituted with 0-3 R^{4a},
C₂-C₆ alkenyl substituted with 0-3 R^{4a},
C₂-C₆ alkynyl substituted with 0-3 R^{4a},
C₃-C₁₀ carbocycle substituted with 0-3 R^{4b},
C₆-C₁₀ aryl substituted with 0-3 R^{4b}, or
5 to 10 membered heterocycle substituted with 0-3 R^{4b};
R^{4a}, at each occurrence, is independently selected from: H,
F, Cl, Br, I, CF₃,
C₃-C₁₀ carbocycle substituted with 0-3 R^{4b},
C₆-C₁₀ aryl substituted with 0-3 R^{4b}, or
5 to 10 membered heterocycle substituted with 0-3 R^{4b};
R^{4b}, at each occurrence, is independently selected from:
H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, C₁-C₄ haloalkoxy, and C₁-C₄ halothioalkoxy;
R⁵ is H, OR¹⁴;
C₁-C₆ alkyl substituted with 0-3 R^{5b};
C₁-C₆ alkoxy substituted with 0-3 R^{5b};
C₂-C₆ alkenyl substituted with 0-3 R^{5b};
C₂-C₆ alkynyl substituted with 0-3 R^{5b};
C₃-C₁₀ carbocycle substituted with 0-3 R^{5c};
C₆-C₁₀ aryl substituted with 0-3 R^{5c}; or
5 to 10 membered heterocycle substituted with 0-3 R^{5c};
provided R⁵ is not hydrogen when R³ is hydrogen;
R^{5a} is H, OH, C₁-C₄ alkyl, C₁-C₄ alkoxy, C₂-C₄ alkenyl, or C₂-C₄ alkenyloxy;
R^{5b}, at each occurrence, is independently selected from:
H, C₁-C₆ alkyl, CF₃, OR¹⁴, Cl, F, Br, I, ═O, CN, NO₂, NR¹⁵R¹⁶;
C₃-C₁₀ carbocycle substituted with 0-3 R^{5c};
C₆-C₁₀ aryl substituted with 0-3 R^{5c}; or
5 to 10 membered heterocycle substituted with 0-3 R^{5c};
R^{5c}, at each occurrence, is independently selected from:
H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, C₁-C₄ haloalkoxy, and C₁-C₄ halothioalkoxy;
alternatively, R⁵ and R^{5a}, and the carbon to which they are attached, may be combined to form a 3-8 membered cycloalkyl moiety substituted with 0-2 R^{5b}; provided that R³ and R^{3a} are not combined to form a 3-8 membered cycloalkyl moiety;
R⁷, at each occurrence, is independently selected from:
H, OH, Cl, F, Br, I, CN, NO₂, CF₃, and C₁-C₄ alkyl;
R^{7a}, at each occurrence, is independently selected from:
H, OH, Cl, F, Br, I, CN, NO₂, CF₃, aryl and C₁-C₄ alkyl;
R^{7b} is independently selected from H and C₁-C₄ alkyl;
L is a bond, C₁-C₄ alkyl, C₂-C₄ alkenyl, C₂-C₄ alkynyl, —(CH₂)ₚ—O—(CH₂)_{q}—, or —(CH₂)ₚ—NR¹⁰—(CH₂)_{q}—;
p is 0, 1, 2, or 3;
q is 0, 1, 2, or 3;
Z is C₃-C₁₀ carbocycle substituted with 0-2 R^{12b};
C₆-C₁₀ aryl substituted with 0-4 R^{12b}; and
5 to 10 membered heterocycle substituted with 0-5 R^{12b}, wherein the heterocycle contains 1, 2, 3 or 4 heteroatoms selected from N, O and S;
R^{12b}, at each occurrence, is independently selected from:
H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, C₁-C₄ haloalkoxy, C₁-C₄ halothioalkoxy, aryl substituted with 0-4 R^{12c};
R^{12c}, at each occurrence, is independently selected from:
H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄haloalkyl, C₁-C₄haloalkoxy, and C₁-C₄halothioalkoxy;
B is a 4 to 8 membered amino-heterocyclic ring, comprising one N atom, 3 to 7 carbon atoms, and optionally, an additional heteroatom selected from —O—, —S—, —S(═O)—, —S(═O)₂—, and —N(R^{LZ})—;
wherein the amino-heterocyclic ring is saturated or partially saturated; and
wherein R^{LZ} is either R¹⁰ or the substituent —L—Z;
R¹⁰ is H, C(═O)R¹⁷, C(═O)OR¹⁷, —(C₁-C₃ alkyl)—C(═O)OR¹⁷, C(═O)NR¹⁸R¹⁹, S(═O)₂NR¹⁸R¹⁹, S(═O)₂R¹⁷;
C₁-C₆ alkyl substituted with 0-2 R^{10a};
C₆-C₁₀ aryl substituted with 0-4 R^{10b};
C₃-C₁₀ carbocycle substituted with 0-3 R^{10b}; or
5 to 10 membered heterocycle optionally substituted with 0-3 R^{10b};
R^{10a}, at each occurrence, is independently selected from:
H, C₁-C₆ alkyl, OR¹⁴, Cl, F, Br, I, ═O, CN, NO₂, NR¹⁵R¹⁶, CF₃, or aryl substituted with 0-4 R^{10b};
R^{10b}, at each occurrence, is independently selected from:
H, OH, C₁-C₆ alkyl, C₁-C₄ alkoxy, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, C₁-C₄ haloalkoxy, and C₁-C₄ halothioalkoxy;
R¹¹, at each occurrence, is independently selected from:
C₁-C₄ alkoxy, Cl, F, Br, I, —OH, CN, NO₂, NR¹⁸R¹⁹, C(═O)R¹⁷, C(═O)OR¹⁷, C(═O)NR¹⁸R¹⁹, S(═O)₂NR¹⁸R¹⁹, CF₃;
C₁-C₆ alkyl substituted with 0-1 R^{11a};
C₆-C₁₀ aryl substituted with 0-3 R^{11b};
C₃-C₁₀ carbocycle substituted with 0-3 R^{11b}; or
5 to 10 membered heterocycle substituted with 0-3 R^{11b};
alternatively, two R¹¹ substituents on the same or adjacent carbon atoms may be combined to form a C₃-C₆ carbocycle or a benzo fused radical, wherein said carbocycle or benzo fused radical is substituted with 0-4 R¹³;
additionally, two R¹¹ substituents on adjacent atoms may be combined to form a 5 to 6 membered heteroaryl fused radical, wherein said 5 to 6 membered heteroaryl fused radical comprises 1 or 2 heteroatoms selected from N, O, and S; wherein said 5 to 6 membered heteroaryl fused radical is substituted with 0-3 R¹³;
R^{11a}, at each occurrence, is independently selected from:
H, C₁-C₆ alkyl, OR¹⁴, Cl, F, Br, I, ═O, CN, NO₂, NR¹⁵R¹⁶, CF₃, or phenyl substituted with 0-3 R^{11b};
R^{11b}, at each occurrence, is independently selected from:
H, OH, Cl, F, Br, I, CN, NO₂, NR¹⁵R¹⁶, CF₃, acetyl,
SCH₃, S(═O)CH₃, S(═O)₂CH₃, C₁-C₆ alkyl, C₁-C₄ alkoxy, C₁-C₄ haloalkyl, C₁-C₄ haloalkoxy, and C₁-C₄ halothioalkoxy;
t is 0, 1, 2 or 3;
R¹³, at each occurrence, is independently selected from: H, OH, C₁-C₆ alkyl, C₁-C₄ alkoxy, Cl, F, Br, I, CN, NO₂, NR¹⁵R¹⁶, and CF₃;
R¹⁴, at each occurrence, is independently selected from: H, phenyl, benzyl, C₁-C₆ alkyl, or C₂-C₆ alkoxyalkyl;
R^{14a} is H, phenyl, benzyl, or C₁-C₄ alkyl;
R¹⁵, at each occurrence, is independently selected from: H, C₁-C₆ alkyl, benzyl, phenethyl, —C(═O)—(C₁-C₆ alkyl), —S(═O)₂—(C₁-C₆ alkyl), and aryl;
R¹⁶, at each occurrence, is independently selected from: H, OH, C₁-C₆ alkyl, benzyl, phenethyl, —C(═O)—(C₁-C₆ alkyl) and —S(═O)₂—(C₁-C₆ alkyl);
alternatively, R¹⁵ and R¹⁶ on the same N atom may be combined to form a 5 to 6 membered heterocyclic fused radical, wherein said 5 to 6 membered heterocyclic fused radical comprises 1 or 2 heteroatoms selected from N and O;
R¹⁷ is H, aryl, aryl—CH₂—, C₁-C₆ alkyl, or C₂-C₆ alkoxyalkyl;
R¹⁸, at each occurrence, is independently selected from:
H, C₁-C₆ alkyl, benzyl, phenethyl, —C(═O)—(C₁-C₆ alkyl) and —S(═O)₂—(C₁-C₆ alkyl);
R¹⁹, at each occurrence, is independently selected from:
H, OH, C₁-C₆ alkyl, phenyl, benzyl, phenethyl, —C(═O)—(C₁-C₆ alkyl) —S(═O)₂—(C₁-C₆ alkyl); and
alternatively, R¹⁸ and R¹⁹ on the same N atom may be combined to form a 5 to 6 membered heterocyclic fused radical, wherein said 5 to 6 membered heterocyclic fused radical comprises 1 or 2 heteroatoms selected from N and O.

2. A compound according to claim 1, wherein:
R³ is —(CR⁷R^{7a})ₙ—R⁴,
—(CR⁷R^{7a})ₙ—S—(CR⁷R^{7a})ₘ—R⁴,
—(CR⁷R^{7a})ₙ—O—(CR⁷R^{7a})ₘ—R⁴,
—(CR⁷R^{7a})ₙ—N(R^{7b})—(CR⁷R^{7a})ₘ—R⁴,
—(CR⁷R^{7a})ₙ—S(═O)—(CR⁷R^{7a})ₘ—R⁴,
—(CR⁷R^{7a})ₙ—S(═O)₂—(CR⁷R^{7a})ₘ—R⁴,
—(CR⁷R^{7a})ₙ—C(═O)—(CR⁷R^{7a})ₘ—R⁴,
—(CR⁷R^{7a})ₙ—NHC(═O)—(CR⁷R^{7a})ₘ—R⁴,
—(CR⁷R^{7a})ₙ—C(═O)NH—(CR⁷R^{7a})ₘ—R⁴,
(CR⁷R^{7a})ₙ—NHS(═O)₂—(CR⁷R^{7a})ₘ—R⁴, or
—(CR⁷R^{7a})ₙ—S(═O)₂NH—(CR⁷R^{7a})ₘ—R⁴;
provided R³ is not hydrogen when R⁵ is hydrogen;
n is 0, 1, 2, or 3;
m is 0, 1, 2, or 3;
R^{3a} is H, OH, methyl, ethyl, propyl, butyl, methoxy, ethoxy, propoxy, or butoxy;
alternatively, R³ and R^{3a}, and the carbon to which they are attached, may be combined to form a 3-8 membered cycloalkyl moiety substituted with 0-1 R^{4b}; provided that R⁵ and R^{5a} are not combined to form a 3-8 membered cycloalkyl moiety;
R⁴ is H, OH, OR^{14a},
C₁-C₆ alkyl substituted with 0-3 R^{4a},
C₂-C₆ alkenyl substituted with 0-3 R^{4a},
C₂-C₆ alkynyl substituted with 0-3 R^{4a},
C₃-C₁₀ carbocycle substituted with 0-3 R^{4b},
C₆-C₁₀ aryl substituted with 0-3 R^{4b}, or
5 to 10 membered heterocycle substituted with 0-3 R^{4b};
R^{4a}, at each occurrence, is independently selected from: H,
F, Cl, Br, I, CF₃,
C₃-C₁₀ carbocycle substituted with 0-3 R^{4b},
C₆-C₁₀ aryl substituted with 0-3 R^{4b}, or
5 to 10 membered heterocycle substituted with 0-3 R^{4b};
R^{4b}, at each occurrence, is independently selected from:
H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, and C₁-C₄ haloalkoxy;
R⁵ is H, OR¹⁴;
C₁-C₆ alkyl substituted with 0-3 R^{5b};
C₁-C₆ alkoxy substituted with 0-3 R^{5b};
C₂-C₆ alkenyl substituted with 0-3 R^{5b};
C₂-C₆ alkynyl substituted with 0-3 R^{5b};
C₃-C₁₀ carbocycle substituted with 0-3 R^{5c};
C₆-C₁₀ aryl substituted with 0-3 R^{5c}; or
5 to 10 membered heterocycle substituted with 0-3R^{5c};
provided R⁵ is not hydrogen when R³ is hydrogen;
R^{5a} is H, OH, methyl, ethyl, propyl, butyl, methoxy, ethoxy, propoxy, butoxy,or allyl;
R^{5b}, at each occurrence, is independently selected from:
H, C₁-C₆ alkyl, CF₃, OR¹⁴, Cl, F, Br, I, ═O, CN, NO₂, NR¹⁵R¹⁶;
C₃-C₁₀ carbocycle substituted with 0-3 R^{5c};
C₆-C₁₀ aryl substituted with 0-3 R^{5c}; or
5 to 10 membered heterocycle substituted with 0-3 R^{5c};
R^{5c}; at each occurrence, is independently selected from:
H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, and C₁-C₄ haloalkoxy;
alternatively, R⁵ and R^{5a}, and the carbon to which they are attached, may be combined to form a 3-8 membered cycloalkyl moiety substituted with 0-1 R^{5b}; provided that R³ and R^{3a} are not combined to form a 3-8 membered cycloalkyl moiety;
R⁷, at each occurrence, is independently selected from:
H, OH, Cl, F, Br, I, CN, NO₂, CF₃, and C₁-C₄ alkyl;
R^{7a}, at each occurrence, is independently selected from:
H, OH, Cl, F, Br, I, CN, NO₂, CF₃, aryl and C₁-C₄ alkyl;
R^{7b} is independently selected from H and C₁-C₄ alkyl;
L is a bond, C₁-C₄ alkyl, C₂-C₄ alkenyl, C₂-C₄ alkynyl, —(CH₂)ₚ—O—(CH₂)_{q}—, or —(CH₂)ₚ—NR¹⁰—(CH₂)_{q}—;
p is 0, 1, 2, or 3;
q is 0, 1, 2, or 3;
Z is C₃-C₁₀ carbocycle substituted with 0-2 R^{12b};
C₆-C₁₀ aryl substituted with 0-4 R^{12b}; and
5 to 10 membered heterocycle substituted with 0-5 R¹²b, wherein the heterocycle contains 1, 2, 3 or 4 heteroatoms selected from N, O and S;
R^{12b}, at each occurrence, is independently selected from:
H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, C₁-C₄ haloalkoxy, aryl substituted with 0-4 R^{12c};
R¹²c, at each occurrence, is independently selected from:
H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, and C₁-C₄ haloalkoxy;
B is a 4 to 8 membered amino-heterocyclic ring, comprising one N atom, 3 to 7 carbon atoms, and optionally, an additional heteroatom selected from —O—, —S—, —S(═O)—, —S(═O)₂—, and —N(R^{LZ})—;
wherein the amino-heterocyclic ring is saturated or partially saturated; and
wherein R^{LZ} is either R¹⁰ or the substituent —L—Z;
R¹⁰ is H, C(═O)R¹⁷, C(═O)OR¹⁷, —(C₁-C₃ alkyl)—C(═O)OR¹⁷, C(═O)NR¹⁸R¹⁹, S(═O)₂NR¹⁸R¹⁹, S(═O)₂R¹⁷;
C₁-C₆ alkyl substituted with 0-1 R^{10a};
C₆-Clo aryl substituted with 0-4 R^{10b};
C₃-C₁₀ carbocycle substituted with 0-3 R^{10b}; or
5 to 10 membered heterocycle optionally substituted with 0-3 R^{10b};
R¹⁰a, at each occurrence, is independently selected from:
H, C₁-C₆ alkyl, OR¹⁴, Cl, F, Br, I, ═O, CN, NO₂, NR¹⁵R¹⁶, CF₃, or phenyl substituted with 0-4 R^{10b};
R^{10b}, at each occurrence, is independently selected from:
H, OH, C₁-C₆ alkyl, C₁-C₄ alkoxy, Cl, F, Br, I, CN, NO₂, NR¹⁵R¹⁶, or CF₃;
R¹¹, at each occurrence, is independently selected from:
C₁-C₄ alkoxy, Cl, F, Br, I, OH, CN, NO₂, NR¹⁸R¹⁹, C(═O)R¹⁷, C(═O)OR¹⁷, C(═O)NR¹⁸R¹⁹, S(═O)₂NR¹⁸R¹⁹, CF₃;
C₁-C₆ alkyl substituted with 0-1 R^{11a};
C₆-C₁₀ aryl substituted with 0-3 R^{11b};
C₃-C₁₀ carbocycle substituted with 0-3 R^{11b}; or
5 to 10 membered heterocycle substituted with 0-3 R^{11b};
alternatively, two R¹¹ substituents on the same or adjacent carbon atoms may be combined to form a C₃-C₆ carbocycle or a benzo fused radical wherein said benzo fused radical is substituted with 0-4 R¹³;
additionally, two R¹¹ substituents on adjacent atoms may be combined to form a 5 to 6 membered heteroaryl fused radical, wherein said 5 to 6 membered heteroaryl fused radical comprises 1 or 2 heteroatoms selected from N, O, and S; wherein said 5 to 6 membered heteroaryl fused radical is substituted with 0-3 R¹³;
R^{11a}, at each occurrence, is independently selected from:
H, C₁-C₆ alkyl, OR¹⁴, Cl, F, Br, I, ═O, CN, NO₂, NR¹⁵R¹⁶, CF₃, or phenyl substituted with 0-3 R^{11b};
R^{11b}, at each occurrence, is independently selected from:
H, OH, Cl, F, Br, I, CN, NO₂, NR¹⁵R¹⁶, CF₃, C₁-C₆ alkyl, C₁-C₄ alkoxy, C₁-C₄ haloalkyl, and C₁-C₄ haloalkoxy;
t is 0, 1, 2 or 3;
R¹³, at each occurrence, is independently selected from:
H, OH, C₁-C₆ alkyl, C₁-C₄ alkoxy, Cl, F, Br, I, CN, NO₂, NR¹⁵R¹⁶, and CF₃;
R¹⁴ is H, phenyl, benzyl, C₁-C₆ alkyl, or C₂-C₆ alkoxyalkyl;
R^{14a} is H, phenyl, benzyl, or C₁-C₄ alkyl;
R¹⁵, at each occurrence, is independently selected from:
H, C₁-C₆ alkyl, benzyl, phenethyl, —C(═O)—(C₁-C₆ alkyl) and —S(═O)₂—(C₁-C₆ alkyl);
R¹⁶, at each occurrence, is independently selected from:
H, OH, C₁-C₆ alkyl, benzyl, phenethyl, —C(═O)—(C₁-C₆ alkyl) —S(═O)₂—(C₁-C₆ alkyl), and phenyl substituted with 0-3 R¹³;
alternatively, R¹⁵ and R¹⁶ on the same N atom may be combined to form a 5 to 6 membered heterocyclic fused radical, wherein said 5 to 6 membered heterocyclic fused radical comprises 1 or 2 heteroatoms selected from N and O;
R¹⁷ is H, aryl, (aryl)CH₂—, C₁-C₆ alkyl, or C₂-C₆ alkoxyalkyl;
R¹⁸ at each occurrence, is independently selected from:
H, C₁-C₆ alkyl, benzyl, phenethyl, —C(═O)—(C₁-C₆ alkyl) and —S(═O)₂—(C₁-C₆ alkyl);
R¹⁹, at each occurrence, is independently selected from:
H, OH, C₁-C₆ alkyl, phenyl, benzyl, phenethyl, —C(═O)—(C₁-C₆ alkyl) and —S(═O)₂—(C₁-C₆ alkyl); and
alternatively, R¹⁸ and R¹⁹ on the same N atom may be combined to form a 5 to 6 membered heterocyclic fused radical, wherein said 5 to 6 membered heterocyclic fused radical comprises 1 or 2 heteroatoms selected from N and O.

3. A compound according to claim 2, wherein:
R³ is —(CHR⁷)ₙ—R⁴,
—(CHR⁷)ₙ—S—(CHR⁷)ₘ—R⁴,
—(CHR⁷)ₙ—O—(CHR⁷)ₘ—R⁴, or
—(CHR⁷)ₙ—N(R^{7b})—(CHR⁷)ₘ—R⁴;
provided R³ is not hydrogen when R⁵ is hydrogen;
n is 0, 1, or 2;
m is 0, 1, or 2;
R^{3a} is H;
alternatively, R³ and R^{3a}, and the carbon to which they are attached, may be combined to form a cyclopropyl, cyclobutyl, cyclopentyl, or cyclohexyl moiety; provided that R⁵ and R^{5a}are not combined to form a cycloalkyl moiety;
R⁴ is H, OH, OR^{14a},
C₁-C₄ alkyl substituted with 0-2 R^{4a},
C₂-C₄ alkenyl substituted with 0-2 R^{4a},
C₂-C₄ alkynyl substituted with 0-2 R^{4a},
C₃-C₆ cycloalkyl substituted with 0-3 R^{4b}, phenyl substituted with 0-3 R^{4b}, or
5 to 6 membered heterocycle substituted with 0-3 R^{4b};
R^{4a}, at each occurrence, is independently selected from: H,
F, Cl, Br, I CF₃,
C₃-C₁₀ carbocycle substituted with 0-3 R^{4b},
phenyl substituted with 0-3 R^{4b}, or
5 to 6 membered heterocycle substituted with 0-3 R^{4b};
R^{4b}, at each occurrence, is independently selected from:
H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, and C₁-C₄ haloalkoxy;
R⁵ is H, OR¹⁴;
C₁-C₆ alkyl substituted with 0-3 R^{5b};
C₂-C₆ alkenyl substituted with 0-3 R^{5b};
C₂-C₆ alkynyl substituted with 0-3 R^{5b};
C₃-C₁₀ carbocycle substituted with 0-3 R^{5c};
C₆-C₁₀ aryl substituted with 0-3 R^{5c}; or
5 to 10 membered heterocycle substituted with 0-3R^{5c};
provided R⁵ is not hydrogen when R³ is hydrogen;
R^{5a}is H;
R^{5b}₇ at each occurrence, is independently selected from:
H, C₁-C₆ alkyl, CF₃, OR¹⁴, Cl, F, Br, I, ═O, CN, NO₂, NR¹⁵R¹⁶;
C₃-C₁₀ carbocycle substituted with 0-3 R^{5c};
C₆-C₁₀ aryl substituted with 0-3 R^{5c}; or
5 to 10 membered heterocycle substituted with 0-3 R^{5c};
R^{5c}, at each occurrence, is independently selected from:
H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, and C₁-C₄ haloalkoxy;
alternatively, R⁵ and R^{5a}, and the carbon to which they are attached, may be combined to form a cyclopropyl, cyclobutyl, cyclopentyl, or cyclohexyl moiety; provided that R³ and R^{3a} are not combined to form a cycloalkyl moiety;
R⁷, at each occurrence, is independently selected from:
H, OH, Cl, F, Br, I, CN, NO₂, CF₃, and C₁-C₄ alkyl;
R^{7b}is independently selected from: H, methyl, ethyl, propyl, and butyl;
L is a bond, —CH₂—, —CH₂CH2—, —CH₂CH₂CH₂—, —CH₂CH═CH₂, —(CH₂)ₚ—O—(CH2)_{q}—, or —(CH₂)ₚ—NR¹⁰—(CH2)_{q}—;
p is 0, 1, 2, or 3;
q is 0, 1, 2, or 3;
Z is C₃-C₁₀ carbocycle substituted with 0-2 R^{12b};
C₆-C₁₀ aryl substituted with 0-4 R^{12b}; and
5 to 10 membered heterocycle substituted with 0-5 R^{12b}, wherein the heterocycle contains 1, 2, 3 or 4 heteroatoms selected from N, O and S;
R^{12b}, at each occurrence, is independently selected from:
H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, C₁-C₄ haloalkoxy, phenyl substituted with 0-3 R^{12c};
R^{12c}, at each occurrence, is independently selected from:
H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, and C₁-C₄ haloalkoxy;
B is a 5, 6, or 7 membered amino-heterocyclic ring, comprising one N atom, 3 to 6 carbon atoms, and optionally, an additional heteroatom —N(R^{LZ})—;
wherein the amino-heterocyclic ring is saturated or partially saturated; and
wherein R^{LZ} is either R¹⁰ or the substituent —L—Z;
R¹⁰ is H, C(═O)R¹⁷, C(═O)OR¹⁷, —(C₁-C₃ alkyl)—C(═O)OR¹⁷,
C(═O)NR¹⁸R¹⁹, S(═O)₂NR¹⁸R¹⁹, S(═O)₂R¹⁷;
C₁-C₆ alkyl substituted with 0-1 R^{10a};
C₆-C₁₀ aryl substituted with 0-4 R^{10b};
C₃-C₁₀ carbocycle substituted with 0-3 R^{10b}; or
5 to 10 membered heterocycle optionally substituted with 0-3 R^{10b};
R^{10a}, at each occurrence, is independently selected from:
H, C₁-C₆ alkyl, OR¹⁴, Cl, F, Br, I, ═O, CN, NO₂, NR¹⁵R¹⁶, CF₃, or phenyl substituted with 0-4 R^{10b};
R^{10b}, at each occurrence, is independently selected from H, OH, C₁-C₆ alkyl, C₁-C₄ alkoxy, Cl, F, Br, I, CN, NO₂, NR¹⁵R¹⁶, or CF₃;
R¹¹, at each occurrence, is independently selected from:
C₁-C₄ alkoxy, Cl, F, NR¹⁸R¹⁹, C(═O)R¹⁷, C(═O)OR¹⁷, C(═O)NR¹⁸RI⁹, S(═O)₂NR¹⁸RI⁹, CF3;
C₁-C₆ alkyl substituted with 0-1 R^{11a};
C₆-C₁₀ aryl substituted with 0-3 R^{11b};
C₃-C₁₀ carbocycle substituted with 0-3 R^{11b}; or
5 to 10 membered heterocycle substituted with 0-3 R^{11b};
alternatively, two R¹¹ substituents on the same or adjacent carbon atoms may be combined to form a C₃-C₆ carbocycle or a benzo fused radical wherein said benzo fused radical is substituted with 0-4 R¹³;
additionally, two R¹¹ substituents on adjacent atoms may be combined to form a 5 to 6 membered heteroaryl fused radical, wherein said 5 to 6 membered heteroaryl fused radical comprises 1 or 2 heteroatoms selected from N, O, and S; wherein said 5 to 6 membered heteroaryl fused radical is substituted with 0-3 R¹³;
R^{11a}, at each occurrence, is independently selected from: H, C₁-C₆ alkyl, OR¹⁴, Cl, F, Br, I, ═O, CN, NO₂, NR¹⁵R¹⁶, CF₃, or phenyl substituted with 0-3 R^{11b};
R^{11b}, at each occurrence, is independently selected from:
H, OH, Cl, F, Br, I, CN, NO₂, NR¹⁵R¹⁶, CF₃, C₁-C₆ alkyl, C₁-C₄ alkoxy, C₁-C₄ haloalkyl, and C₁-C₄ haloalkoxy;
t is 0, 1, 2 or 3;
R¹³, at each occurrence, is independently selected from: H, OH, C₁-C₆ alkyl, C₁-C₄ alkoxy, Cl, F, Br, I, CN, NO₂, NR¹⁵R¹⁶, and CF₃;
R¹⁴ is H, phenyl, benzyl, C₁-C₆ alkyl, or C₂-C₆ alkoxyalkyl;
R^{14a} is H, phenyl, benzyl, or C₁-C₄ alkyl;
R¹⁵, at each occurrence, is independently selected from:
H, C₁-C₆ alkyl, benzyl, phenethyl, —C(═O)—(C₁-C₆ alkyl), —S(═O)₂—(C₁-C₆ alkyl), and aryl;
R¹⁶ at each occurrence, is independently selected from:
H, OH, C₁-C₆ alkyl, benzyl, phenethyl, —C(═O)—(C₁-C₆ alkyl) and —S(═O)₂—(C₁-C₆ alkyl);
alternatively, R¹⁵ and R¹⁶ on the same N atom may be combined to form a 5 to 6 membered heterocyclic fused radical, wherein said 5 to 6 membered heterocyclic is selected from pyrrolidonyl, piperidonyl, piperazinyl, and morpholinyl;
R¹⁷ is H, aryl, (aryl)CH₂—, C₁-C₆ alkyl, or C₂-C₆ alkoxyalkyl;
R¹⁸, at each occurrence, is independently selected from:
H, C₁-C₆ alkyl, benzyl, phenethyl, —C(═O)—(C₁-C₆ alkyl) and —S(═O)₂—(C₁-C₆ alkyl);
R¹⁹, at each occurrence, is independently selected from:
H, OH, C₁-C₆ alkyl, phenyl, benzyl, phenethyl, —C(═O)—(C₁-C₆ alkyl) and —S(═O)₂—(C₁-C₆ alkyl); and
alternatively, R¹⁸ and R¹⁹ on the same N atom may be combined to form a 5 to 6 membered heterocyclic fused radical, wherein said 5 to 6 membered heterocyclic is selected from pyrrolidonyl, piperidonyl, piperazinyl, and morpholinyl.

4. A compound according to claim 3, of Formula (Ic): 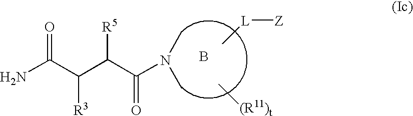
or a pharmaceutically acceptable salt or prodrug thereof, wherein:
R³ is C₁-C₄ alkyl substituted with 0-2 R^{4a}, C₂-C₄ alkenyl substituted with 0-2 R^{4a}, or C₂-C₄ alkynyl substituted with 0-1 R^{4a};
R^{4a}, at each occurrence, is independently selected from: H, F, Cl, CF₃,
C₃-C₆ cycloalkyl substituted with 0-3 R^{4b},
phenyl substituted with 0-3 R^{4b}, or
5 to 6 membered heterocycle substituted with 0-3 R^{4b};
R^{4b}, at each occurrence, is independently selected from:
H, OH, Cl, F, Br, I, CN, NO₂, NR¹⁵R¹⁶, CF₃, acetyl, SCH₃, S(═O)CH₃, S(═O)₂CH₃, C₁-C₄ alkyl, C₁-C₃ alkoxy, C₁-C₂ haloalkyl, and C₁-C₂ haloalkoxy;
R⁵ is C₁-C₆ alkyl substituted with 0-3 R^{5b};
C₂-C₆ alkenyl substituted with 0-2 R^{5b}; or
C₂-C₆ alkynyl substituted with 0-2 R^{5b};
R^{5b}, at each occurrence, is independently selected from:
H, methyl, ethyl, propyl, butyl, CF₃, OR¹⁴, ═O;
C₃-C₆ cycloalkyl substituted with 0-2 R^{5c};
phenyl substituted with 0-3 R^{5c}; or
5 to 6 membered heterocycle substituted with 0-2 R^{5c};
R^{5c}, at each occurrence, is independently selected from:
H, OH, Cl, F, Br, I, CN, NO₂, NR¹⁵R¹⁶, CF₃, acetyl, SCH₃, S(═O)CH₃, S(═O)₂CH₃, C₁-C₄ alkyl, C₁-C₃ alkoxy, C₁-C₂ haloalkyl, and C₁-C₂ haloalkoxy;
L is a bond, —CH₂—, —CH₂CH₂—, —CH₂CH₂CH₂—, —CH₂CH═CH₂, —(CH₂)ₚ—O—(CH₂)_{q}—, or —(CH₂)ₚ—NR¹⁰—(CH₂)_{q}—;
p is 0, 1, 2, or 3;
q is 0, 1, or 2;
Z is C₃-C₁₀ carbocycle substituted with 0-2 R^{12b};
C₆-C₁₀ aryl substituted with 0-4 R^{12b}; and
5 to 10 membered heterocycle substituted with 0-5 R^{12b}, wherein the heterocycle contains 1, 2, 3 or 4 heteroatoms selected from N, O and S;
R^{12b}, at each occurrence, is independently selected from:
H, OH, Cl, F, NR¹⁵R¹⁶, CF₃, acetyl, SCH₃, S(═O)CH₃, S(═O)₂CH₃, C₁-C₄ alkyl, C₁-C₃ alkoxy, C₁-C₂ haloalkyl, C₁-C₂ haloalkoxy, phenyl substituted with 0-3 R^{12c};
R^{12c}, at each occurrence, is independently selected from:
H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, and C₁-C₄ haloalkoxy;
B is a 5 or 6 membered amino-heterocyclic ring, comprising one N atom, 3 to 5 carbon atoms, and optionally, an additional heteroatom —N(R^{LZ})—;
wherein the amino-heterocyclic ring is saturated or partially saturated; and
wherein R^{LZ} is either R¹⁰ or the substituent —L—Z;
R¹⁰ is H, C(═O)R¹⁷, C(═O)OR¹⁷, —(C₁-C₃ alkyl)—C(═O)OR¹⁷;
C₁-C₄ alkyl substituted with 0-1 R^{10a};
phenyl substituted with 0-4 R^{10b};
C₃-C₆ carbocycle substituted with 0-3 R^{10b}; or
5 to 6 membered heterocycle optionally substituted with 0-3 R^{10b};
R^{10a}, at each occurrence, is independently selected from:
H, C₁-C₄ alkyl, OR¹⁴, Cl, F, Br, I, ═O, CN, NO₂, NR¹⁵R¹⁶, CF₃, or phenyl substituted with 0-4 R^{10b};
R^{10b}, at each occurrence, is independently selected from:
H, OH, C₁-C₄ alkyl, C₁-C₃ alkoxy, Cl, F, Br, I, CN, NO₂, NR¹⁵R¹⁶, or CF₃;
R¹¹, at each occurrence, is independently selected from:
C₁-C₄ alkoxy, Cl, F, OH, NR¹⁸R¹⁹, C(═O)R¹⁷, C(═O)OR¹⁷, CF₃;
C₁-C₄ alkyl substituted with 0-1 R^{11a};
phenyl substituted with 0-3 R^{11b};
C₃-C₆ carbocycle substituted with 0-3 R^{11b}; or
5 to 6 membered heterocycle substituted with 0-3 R^{11b};
alternatively, two R¹¹ substituents on adjacent carbon atoms may be combined to form a cyclopropyl, cyclobutyl, cyclopentyl, cyclohexyl, or a benzo fused radical;
R^{11a}, at each occurrence, is independently selected from:
H, C₁-C₄ alkyl, OR¹⁴, F, ═O, NR¹⁵R¹⁶, CF₃, or phenyl substituted with 0-3 R^{11b};
R^{11b}, at each occurrence, is independently selected from:
H, OH, Cl, F, NR¹⁵R¹⁶, CF₃, C₁-C₄ alkyl, C₁-C₃ alkoxy, C₁-C₂ haloalkyl, and C₁-C₂ haloalkoxy;
t is 0, 1, or 2;
R¹³, at each occurrence, is independently selected from:
H, OH, C₁-C₆ alkyl, C₁-C₄ alkoxy, Cl, F, Br, I, CN, NO₂, NR¹⁵R¹⁶, and CF₃;
R¹⁴ is H, phenyl, benzyl, C₁-C₄ alkyl, or C₂-C₄ alkoxyalkyl;
R¹⁵, at each occurrence, is independently selected from:
H, C₁-C₄ alkyl, benzyl, phenethyl, —C(═O)—(C₁-C₄ alkyl), —S(═O)₂—(C₁-C₄ alkyl), and aryl;
R¹⁶ at each occurrence, is independently selected from:
H, OH, C₁-C₄ alkyl, benzyl, phenethyl, —C(═O)—(C₁-C₄ alkyl) and —S(═O)₂—(C₁-C₄ alkyl);
alternatively, R¹⁵ and R¹⁶ on the same N atom may be combined to form a 5 to 6 membered heterocyclic fused radical, wherein said 5 to 6 membered heterocyclic is selected from pyrrolidonyl, piperidonyl, piperazinyl, and morpholinyl;
R¹⁷ is H, phenyl, benzyl, 4-fluorophenyl, 4-chlorophenyl, 4-methylphenyl, 4-trifluorophenyl, (4-fluorophenyl)methyl, (4-chlorophenyl)methyl, (4-methylphenyl)methyl, (4-trifluorophenyl)methyl, methyl, ethyl, propyl, butyl, methoxymethyl, methyoxyethyl, ethoxymethyl, or ethoxyethyl;
R¹⁸, at each occurrence, is independently selected from:
H, methyl, ethyl, propyl, butyl, phenyl, benzyl, and phenethyl;
R¹⁹, at each occurrence, is independently selected from:
H, methyl, and ethyl; and
alternatively, R¹⁸ and R¹⁹ on the same N atom may be combined to form a 5 to 6 membered heterocyclic fused radical, wherein said 5 to 6 membered heterocyclic is selected from pyrrolidonyl, piperidonyl, piperazinyl, and morpholinyl.

5. A compound according to claim 4, of Formula (Ic): 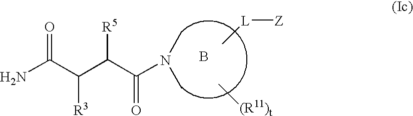
or a pharmaceutically acceptable salt or prodrug thereof, wherein:
R³ is C₁-C₄ alkyl, C₂-C₄ alkenyl, or C₂-C₄ alkynyl;
R⁵ is C₁-C₆ alkyl, C₂-C₆ alkenyl, or C₂-C₆ alkynyl;
L is a bond, —CH₂—, —CH₂CH₂—, —CH₂CH₂CH₂—, —CH₂CH═CH₂, —(CH₂)ₚ—O—(CH₂)_{q}—, or —(CH₂)ₚ—NR¹⁰—(CH₂)_{q}—;
p is 0, 1, 2, or 3;
q is 0, 1, or 2;
Z is C₃-C₁₀ carbocycle substituted with 0-2 R^{12b};
C₆-C₁₀ aryl substituted with 0-4 R^{12b}; and
5 to 10 membered heterocycle substituted with 0-5 R^{12b}, wherein the heterocycle contains 1, 2, 3 or 4 heteroatoms selected from N, O and S;
R^{12b}, at each occurrence, is independently selected from:
H, OH, Cl, F, NR¹⁵R¹⁶, CF₃, acetyl, SCH₃, S(═O)CH₃, S(═O)₂CH₃, methyl, ethyl, propyl, butyl, methoxy, ethoxy, propoxy, C₁-C₂ haloalkyl, C₁-C₂ haloalkoxy, phenyl substituted with 0-3 R^{12c};
R^{12c}, at each occurrence, is independently selected from:
H, OH, Cl, F, Br, I, CN, NO₂, NR¹⁵R¹⁶, CF₃, acetyl, SCH₃, S(═O)CH₃, S(═O)₂CH₃, C₁-C₆ alkyl, C₁-C₄ alkoxy, C₁-C₄ haloalkyl, and C₁-C₄ haloalkoxy;
B is a 6 membered amino-heterocyclic ring, comprising one N atom, 4 or 5 carbon atoms, and optionally, an additional heteroatom —N(R^{LZ})—;
wherein the amino-heterocyclic ring is saturated or partially saturated; and
wherein R^{LZ} is either R¹⁰ or the substituent —L—Z;
R¹⁰ is H, C(═O)R¹⁷, C(═O)OR¹⁷, —(C₁-C₃ alkyl)—C(═O)OR¹⁷;
C₁-C₄ alkyl substituted with 0-1 R^{10a};
phenyl substituted with 0-4 R^{10b};
C₃-C₆ carbocycle substituted with 0-3 R^{10b}; or
5 to 6 membered heterocycle optionally substituted with 0-3 R^{10b};
R^{10a}, at each occurrence, is independently selected from:
H, C₁-C₄ alkyl, OR¹⁴, Cl, F, Br, I, ═O, CN, NO₂, NR¹⁵R¹⁶, CF₃, or phenyl substituted with 0-4 R^{10b};
R^{10b}, at each occurrence, is independently selected from:
H, OH, C₁-C₄ alkyl, C₁-C₃ alkoxy, Cl, F, Br, I, CN, NO₂, NR¹⁵R¹⁶, or CF₃;
R¹¹, at each occurrence, is independently selected from:
C₁-C₄ alkoxy, Cl, F, OH, NR¹⁸R¹⁹, C(═O)R¹⁷, C(═O)OR¹⁷, CF₃;
C₁-C₄ alkyl substituted with 0-1 R^{11a};
phenyl substituted with 0-3 R^{11b};
C₃-C₆ carbocycle substituted with 0-3 R^{11b}; or
5 to 6 membered heterocycle substituted with 0-3 R^{11b};
R^{11a}, at each occurrence, is independently selected from:
H, C₁-C₄ alkyl, OR¹⁴, F, ═O, NR¹⁵R¹⁶, CF₃, or phenyl substituted with 0-3 R^{11b};
R^{11b}, at each occurrence, is independently selected from:
H, OH, Cl, F, NR¹⁵R¹⁶, CF₃, C₁-C₄ alkyl, C₁-C₃ alkoxy, C₁-C₂ haloalkyl, and C₁-C₂ haloalkoxy;
t is 0, 1, or 2;
R¹³, at each occurrence, is independently selected from:
H, OH, C₁-C₆ alkyl, C₁-C₄ alkoxy, Cl, F, Br, I, CN, NO₂, NR¹⁵R¹⁶, and CF₃;
R¹⁴ is H, phenyl, benzyl, methyl, ethyl, propyl, butyl;
R¹⁵, at each occurrence, is independently selected from:
H, methyl, ethyl, propyl, butyl, and phenyl substituted with 0-3 substituents selected from OH, OCH₃, Cl, F, Br, I, CN, NO₂, NH₂, N(CH₃)H, N(CH₃)₂, CF₃, OCF₃, C(═O)CH₃, SCH₃, S(═O)CH₃, S(═O)₂CH₃, CH₃, CH₂CH₃, CO₂H, and CO₂CH₃;
R¹⁶, at each occurrence, is independently selected from:
H, OH, C₁-C₄ alkyl, benzyl, phenethyl, —C(═O)—(C₁-C₄ alkyl) and —S(═O)₂—(C₁-C₄ alkyl);
alternatively, R¹⁵ and R¹⁶ on the same N atom may be combined to form a 5 to 6 membered heterocyclic fused radical, wherein said 5 to 6 membered heterocyclic is selected from pyrrolidonyl, piperidonyl, piperazinyl, and morpholinyl;
R¹⁷ is H, phenyl, benzyl, 4-fluorophenyl, 4-chlorophenyl, 4-methylphenyl, 4-trifluorophenyl, (4-fluorophenyl)methyl, (4-chlorophenyl)methyl, (4-methylphenyl)methyl, (4-trifluorophenyl)methyl, methyl, ethyl, propyl, butyl, methoxymethyl, methyoxyethyl, ethoxymethyl, or ethoxyethyl;
R¹⁸, at each occurrence, is independently selected from:
H, methyl, ethyl, propyl, butyl, phenyl, benzyl, and phenethyl;
R¹⁹, at each occurrence, is independently selected from:
H, methyl, ethyl, and
alternatively, R¹⁸ and R¹⁹ on the same N atom may be combined to form a 5 to 6 membered heterocyclic fused radical, wherein said 5 to 6 membered heterocyclic is selected from pyrrolidonyl, piperidonyl, piperazinyl, and morpholinyl.

6. A compound according to claim 4, of Formula (Ib): 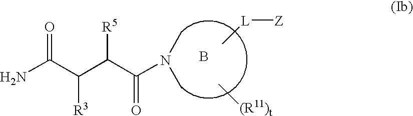
or a pharmaceutically acceptable salt or prodrug thereof, wherein:
R³ is —CH₃, —CH₂CH₃, —CH₂CH₂CH₃, —CH₂CH₂CH₂CH₃, —CH₂(CH₃)₂, —CH(CH₃)CH₂CH₃, —CH₂CH(CH₃)₂, —CH₂C(CH₃)₃, —CF₃, —CH₂CF₃, —CH₂CH₂CF₃, —CH₂CH₂CH₂CF₃;
—CH═CH₂, —CH₂CH═CH₂, —CH₂C(CH₃)═CH₂, —CH₂CH═C(CH₃)₂, —CH₂CH₂CH═CH₂, —CH₂CH₂C(CH₃)═CH₂, —CH₂CH₂CH═C(CH₃)₂, cis-CH₂CH═CH(CH₃), cis-CH₂CH₂CH═CH(CH₃), trans-CH₂CH═CH(CH₃), trans-CH₂CH₂CH═CH(CH₃);
—C≡CH, —CH₂C≡CH, —CH₂C≡C(CH₃);
cyclopropyl-CH₂—, cyclobutyl-CH₂—, cyclopentyl-CH₂—, cyclohexyl-CH₂—, cyclopropyl-CH₂CH₂—, cyclobutyl-CH₂CH₂—, cyclopentyl-CH₂CH₂—, cyclohexyl-CH₂CH₂—; phenyl-CH₂—, (2-F-phenyl)CH₂—, (3-F-phenyl)CH₂—, (4-F-phenyl)CH₂—, (2-Cl-phenyl)CH₂—, (3-Cl-phenyl)CH₂—, (4-Cl-phenyl)CH₂—, (2,3-diF-phenyl)CH₂—, (2,4-diF-phenyl)CH₂—, (2,5-diF-phenyl)CH₂—, (2,6-diF-phenyl)CH₂—, (3,4-diF-phenyl)CH₂—, (3,5-diF-phenyl)CH₂—, (2,3-diCl-phenyl)CH₂—, (2,4-diCl-phenyl)CH₂—, (2,5-diCl-phenyl)CH₂—, (2,6-diCl-phenyl)CH₂—, (3,4-diCl-phenyl)CH₂—, (3,5-diCl-phenyl)CH₂—, (3-F-4-Cl-phenyl)CH₂—, (3-F-5-Cl-phenyl)CH₂—, (3-Cl-4-F-phenyl)CH₂—, phenyl-CH₂CH₂—, (2-F-phenyl)CH₂CH₂—, (3-F-phenyl)CH₂CH₂—, (4-F-phenyl)CH₂CH₂—, (2-Cl-phenyl)CH₂CH₂—, (3-Cl-phenyl)CH₂CH₂—, (4-Cl-phenyl)CH₂CH₂—, (2,3-diF-phenyl)CH₂CH₂—, (2,4-diF-phenyl)CH₂CH₂—, (2,5-diF-phenyl)CH₂CH₂—, (2,6-diF-phenyl)CH₂CH₂—, (3,4-diF-phenyl)CH₂CH₂—, (3,5-diF-phenyl)CH₂CH₂—, (2,3-diCl-phenyl)CH₂CH₂13 , (2,4-diCl-phenyl)CH₂CH₂—, (2,5-diCl-phenyl)CH₂CH₂—, (2,6-diCl-phenyl)CH₂CH₂—, (3,4-diCl-phenyl)CH₂CH₂—, (3,5-diCl-phenyl)CH₂CH₂—, (3-F-4-Cl-phenyl)CH₂CH₂—, or (3-F-5-Cl-phenyl)CH₂CH₂—;
R⁵ is —CH₃, —CH₂CH₃, —CH₂CH₂CH₃, —CH₂(CH₃)₂, —CH₂CH₂CH₂CH₃, —CH(CH₃)CH₂CH₃, —CH₂CH(CH₃)₂, —CH₂C(CH₃)₃, —CH₂CH₂CH₂CH₂CH₃, —CH(CH₃)CH₂CH₂CH₃, —CH₂CH(CH₃)CH₂CH₃, —CH₂CH₂CH(CH₃)₂, —CH(CH₂CH₃)₂, —CF₃, —CH₂CF₃, —CH₂CH₂CF₃, —CH₂CH₂CH₂CF₃, —CH₂CH₂CH₂CH₂CF₃, —CH═CH₂, —CH₂CH═CH₂, —CH═CHCH₃, —CH₂C(CH₃)═CH₂, cis-CH₂CH═CH(CH₃), trans-CH₂CH═CH(CH₃), trans-CH₂CH═CH(C₆H₅), —CH₂CH═C(CH₃)₂, cis-CH₂CH═CHCH₂CH₃, trans-CH₂CH═CHCH₂CH₃, cis-CH₂CH₂CH═CH(CH₃), trans-CH₂CH₂CH═CH(CH₃), trans-CH₂CH═CHCH₂(C₆H₅), —C≡CH, —CH₂C≡CH, —CH₂C≡C(CH₃), —CH₂C≡C (C₆H₅), —CH₂CH₂C≡CH, —CH₂CH₂C≡C(CH₃), —CH₂CH₂C≡C (C₆H₅), —CH₂CH₂CH₂C≡CH, —CH₂CH₂CH₂C≡C(CH₃), —CH₂CH₂CH₂C≡C(C₆H₅), cyclopropyl-CH₂—, cyclobutyl-CH₂—, cyclopentyl-CH₂—, cyclohexyl-CH₂—, (2-CH₃-cyclopropyl)CH₂—, (3-CH₃-cyclobutyl)CH₂—, cyclopropyl-CH₂CH₂—, cyclobutyl-CH₂CH₂—, cyclopentyl-CH₂CH₂—, cyclohexyl-CH₂CH₂—, (2-CH₃-cyclopropyl)CH₂CH₂—, (3-CH₃-cyclobutyl)CH₂CH₂—, phenyl-CH₂—, (2-F-phenyl)CH₂—, (3-F-phenyl)CH₂—, (4-F-phenyl)CH₂—, furanyl-CH₂—, thienyl-CH₂—, pyridyl-CH₂—, 1-imidazolyl-CH₂—, oxazolyl-CH₂—, isoxazolyl-CH₂—, phenyl-CH₂CH₂—, (2-F-phenyl)CH₂CH₂—, (3-F-phenyl)CH₂CH₂—, (4-F-phenyl)CH₂CH₂—, furanyl-CH₂CH₂—, thienyl-CH₂CH₂—, pyridyl-CH₂CH₂—, 1-imidazolyl-CH₂CH₂—, oxazolyl-CH₂CH₂—, or isoxazolyl-CH₂CH₂—;
L is a bond, —CH₂—, —CH₂CH₂—, —CH₂CH₂CH₂—, —CH₂CH═CH₂, O, —CH₂O—, —(CH₂)₂—O—, —(CH₂)₃—O—, —(CH₂)—O—(CH₂)₂—, —(CH₂)₂—O—(CH₂)—, —(CH₂)₂—O—(CH₂)₂—, NH, NMe, —CH₂NH—, —(CH₂)₂—NH—, —(CH₂)₃—NH—, —(CH₂)—NH—(CH₂)₂—, —(CH₂)₂—NH—(CH₂)—, —(CH₂)₂—NH—(CH₂)₂—, and —N(benzoyl)-;
z is cyclopropyl, cyclobutyl, cyclopentyl, cyclohexyl, phenyl 2-F-phenyl, 3-F-phenyl, 4-F-phenyl, 2-Cl-phenyl, 3-Cl-phenyl, 4-Cl-phenyl, 2,3-diF-phenyl, 2,4-diF-phenyl, 2,5-diF-phenyl, 2,6-diF-phenyl, 3,4-diF-phenyl, 3,5-diF-phenyl, 2,3-diCl-phenyl, 2,4-diCl-phenyl, 2,5-diCl-phenyl, 2,6-diCl-phenyl, 3,4-diCl-phenyl, 3,5-diCl-phenyl, 2,3-diMe-phenyl, 2,4-diMe-phenyl, 2,5-diMe-phenyl, 2,6-diMe-phenyl, 3,4-diMe-phenyl, 3,5-diMe-phenyl, 2,3-diMeO-phenyl, 2,4-diMeO-phenyl, 2,5-diMeO-phenyl, 2,6-diMeO-phenyl, 3,4-diMeO-phenyl, 3,5-diMeO-phenyl, 3-F-4-Cl-phenyl, 3-F-5-Cl-phenyl, 3-Cl-4-F-phenyl, 2-MeO-phenyl, 3-MeO-phenyl, 4-MeO-phenyl, 2-EtO-phenyl, 3-EtO-phenyl, 4-EtO-phenyl, 2-Me-phenyl, 3-Me-phenyl, 4-Me-phenyl, 2-Et-phenyl, 3-Et-phenyl, 4-Et-phenyl, 2-CF₃-phenyl, 3-CF₃-phenyl, 4-CF₃-phenyl, 2-NO₂-phenyl, 3-NO₂-phenyl, 4-NO₂-phenyl, 2-CN-phenyl, 3-CN-phenyl, 4-CN-phenyl, 2-MeS-phenyl, 3-MeS-phenyl, 4-MeS-phenyl, 2-CF₃O-phenyl, 3-CF₃O-phenyl, 4-CF₃O-phenyl, 2-Me-5-Cl-phenyl, 3-CF₃-4-Cl-phenyl, 3-CF₃-5-F-phenyl, 3-MeO-4-Me-phenyl, furanyl, thienyl, pyrid-2-yl, pyrid-3-yl, pyrid-4-yl, pyrimidyl, pyrazinyl, 2-Me-pyridyl, 3-Me-pyridyl, 3-CF₃-pyrid-2-yl, 5-CF₃-pyrid-2-yl, 4-Me-pyridyl, pyrrolidinyl, 1-imidazolyl, oxazolyl, isoxazolyl, 1-benzimidazolyl, 2-keto-1-benzimidazolyl, 4-benzo[1,3]dioxol-5-yl, morpholino, N-piperidyl, 4-piperidyl, naphthyl, 4(phenyl)phenyl-, 4(4-CF₃-phenyl)phenyl-, 3,5-bis-CF₃-phenyl-, 4-iPr-phenyl-, N-piperidino-CH₂—, 1-Me-pyrrolidin-2-yl, and 1-pyrrolidinyl;
B is a 5 or 6 membered amino-heterocyclic ring, comprising one N atom, 3 to 5 carbon atoms, and optionally, an additional heteroatom —N(R^{LZ})—;
wherein the amino-heterocyclic ring is saturated or partially saturated; and
wherein R^{LZ} is either R¹⁰ or the substituent —L—Z;
R¹⁰ is H, methyl, ethyl, phenyl, benzyl, phenethyl, 4-F-phenyl, (4-F-phenyl)CH₂—, (4-F-phenyl)CH₂CH₂—, 4-Cl-phenyl, (4-Cl-phenyl)CH₂—, (4-Cl-phenyl)CH₂CH₂—, 4-CH₃-phenyl, (4-CH₃-phenyl)CH₂—, (4-CH₃-phenyl)CH₂CH₂=13 , 4-CF₃-phenyl, (4-CF₃-phenyl)CH₂—, (4-CF₃-phenyl)CH₂CH₂—, —CH₂C(═O)Et, —C(═O)Me, or 4-Cl-benzhydryl;
R¹¹, at each occurrence, is independently selected from:
H, OH, methyl, ethyl, —CN, —C(═O)Me, —C(═O)OEt, —C(═O)Et, —CH₂OH, —C(═O)NH₂, —C(═O)OH, —C(═O)N(Et)₂, phenyl, benzyl, phenethyl, 4-F-phenyl, (4-F-phenyl)CH₂—, (4-F-phenyl)CH₂CH₂—, 4-Cl-phenyl, (4-Cl-phenyl)CH₂—, (4-Cl-phenyl)CH₂CH₂—, 4-CH₃-phenyl, (4-CH₃-phenyl)CH₂—, (4-CH₃-phenyl)CH₂CH₂—, 4-CF₃-phenyl, (4-CF₃-phenyl)CH₂—, (4-CF₃-phenyl)CH₂CH₂—, and —N(Me)₂—,; and
t is 0, 1, or 2;
alternatively, two R¹¹ substituents on the same or adjacent carbon atoms may be combined to form a cyclopropyl, cyclobutyl, cyclopentyl, cyclohexyl, or a benzo fused radical.

7. A compound according to claim 4, of Formula (Ib): 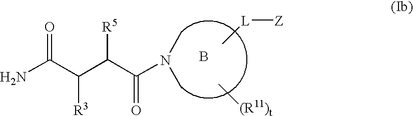
or a pharmaceutically acceptable salt or prodrug thereof, wherein:
R³ is —CH₂CH₂CH₃, —CH₂CH₂CH₂CH₃, —CH₂(CH₃)₂, —CH₂CH(CH₃)₂, —CH₂CH═CH₂, —CH₂CH₂CH═CH₂, —CH₂CH₂CH═C(CH₃)₂, cis-CH₂CH═CH(CH₃), cis-CH₂CH₂CH═CH(CH₃), trans-CH₂CH═CH(CH₃), trans-CH₂CH₂CH═CH(CH₃); cyclopropyl-CH₂—, cyclobutyl-CH₂—, cyclopentyl-CH₂—, cyclohexyl-CH₂—, cyclopropyl-CH₂CH₂—, cyclobutyl-CH₂CH₂—, cyclopentyl-CH₂CH₂—, or cyclohexyl-CH₂CH₂—;
R⁵ is —CH₂(CH₃)₂, —CH₂CH₂CH₂CH₃, —CH(CH₃)CH₂CH₃, —CH₂CH(CH₃)₂, —CH₂C(CH₃)₃, —CH₂CH₂CH₂CH₂CH₃, —CH(CH₃)CH₂CH₂CH₃, —CH₂CH(CH₃)CH₂CH₃, —CH₂CH₂CH(CH₃)₂, —CH(CH₂CH₃)₂, —CH₂CH═CH₂, —CH₂C(CH₃)═CH₂, cis-CH₂CH═CH(CH₃), trans-CH₂CH═CH(CH₃), —CH₂CH═C(CH₃)₂, cyclopropyl-CH₂—, cyclobutyl-CH₂—, cyclopentyl-CH₂—, cyclohexyl-CH₂—, (2-CH₃-cyclopropyl)CH₂—, or (3-CH₃-cyclobutyl)CH₂—,
L is a bond, —CH₂—, —CH₂CH₂—, —CH₂CH₂CH₂—, —CH₂CH═CH₂, O, —CH₂O—, —(CH₂)₂—O—, —(CH₂)₃—O—, —(CH₂)—O—(CH₂)₂—, —(CH₂)₂—O—(CH₂)—, —(CH₂)₂—O—(CH₂)₂—, NH, NMe, —CH₂NH—, —(CH₂)₂—NH—, —(CH₂)₃—NH—, —(CH₂)—NH—(CH₂)₂—, —(CH₂)₂—NH—(CH₂)—, —(CH₂)₂—NH—(CH₂)₂—, and —N(benzoyl)-;
Z is cyclopropyl, cyclobutyl, cyclopentyl, cyclohexyl, phenyl 2-F-phenyl, 3-F-phenyl, 4-F-phenyl, 2-Cl-phenyl, 3-Cl-phenyl, 4-Cl-phenyl, 2,3-diF-phenyl, 2,4-diF-phenyl, 2,5-diF-phenyl, 2,6-diF-phenyl, 3,4-diF-phenyl, 3,5-diF-phenyl, 2,3-diCl-phenyl, 2,4-diCl-phenyl, 2,5-diCl-phenyl, 2,6-diCl-phenyl, 3,4-diCl-phenyl, 3,5-diCl-phenyl, 2,3-diMe-phenyl, 2,4-diMe-phenyl, 2,5-diMe-phenyl, 2,6-diMe-phenyl, 3,4-diMe-phenyl, 3,5-diMe-phenyl, 2,3-diMeO-phenyl, 2,4-diMeO-phenyl, 2,5-diMeO-phenyl, 2,6-diMeO-phenyl, 3,4-diMeO-phenyl, 3,5-diMeO-phenyl, 3-F-4-Cl-phenyl, 3-F-5-Cl-phenyl, 3-Cl-4-F-phenyl, 2-MeO-phenyl, 3-MeO-phenyl, 4-MeO-phenyl, 2-EtO-phenyl, 3-EtO-phenyl, 4-EtO-phenyl, 2-Me-phenyl, 3-Me-phenyl, 4-Me-phenyl, 2-Et-phenyl, 3-Et-phenyl, 4-Et-phenyl, 2-CF₃-phenyl, 3-CF₃-phenyl, 4-CF₃-phenyl, 2-NO₂-phenyl, 3-NO₂-phenyl, 4-NO₂-phenyl, 2-CN-phenyl, 3-CN-phenyl, 4-CN-phenyl, 2-MeS-phenyl, 3-MeS-phenyl, 4-MeS-phenyl, 2-CF₃O-phenyl, 3-CF₃O-phenyl, 4-CF₃O-phenyl, 2-Me-5-Cl-phenyl, 3-CF₃-4-Cl-phenyl, 3-CF₃-5-F-phenyl, 3-MeO-4-Me-phenyl, furanyl, thienyl, pyrid-2-yl, pyrid-3-yl, pyrid-4-yl, pyrimidyl, pyrazinyl, 2-Me-pyridyl, 3-Me-pyridyl, 3-CF₃-pyrid-2-yl, 5-CF₃-pyrid-2-yl, 4-Me-pyridyl, pyrrolidinyl, 1-imidazolyl, oxazolyl, isoxazolyl, 1-benzimidazolyl, 2-keto-1-benzimidazolyl, 4-benzo[1,3]dioxol-5-yl, morpholino, N-piperidyl, 4-piperidyl, naphthyl, 4(phenyl)phenyl-, 4(4-CF₃-phenyl)phenyl-, 3,5-bis-CF₃-phenyl-, 4-iPr-phenyl-, N-piperidino-CH₂—, 1-Me-pyrrolidin-2-yl, and 1-pyrrolidinyl;
B is a 5 or 6 membered amino-heterocyclic ring, comprising one N atom, 3 to 5 carbon atoms, and optionally, an additional heteroatom —N(R^{LZ})—;
wherein the amino-heterocyclic ring is saturated or partially saturated; and
wherein R^{LZ} is the substituent —L—Z;
R¹¹, at each occurrence, is independently selected from:
H, OH, methyl, ethyl, —CN, —C(═O)Me, —C(═O)OEt, —C(═O)Et, —CH₂OH, —C(═O)NH₂, —C(═O)OH, —C(═O)N(Et)₂, and —N(Me)₂—;
t is 0 or 1.

8. A compound according to claim 1, wherein:
B is

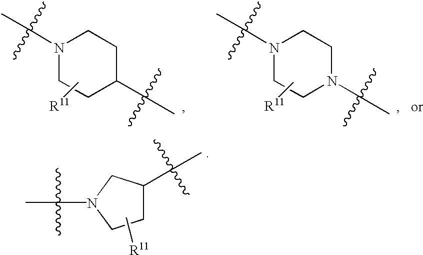

9. A compound according to claim 2, wherein:
B is

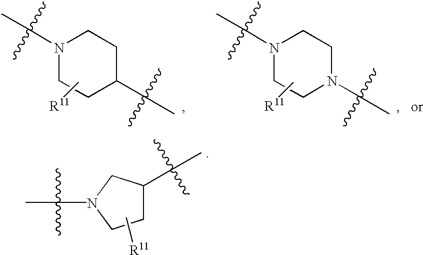

10. A compound according to claim 3, wherein:
B is

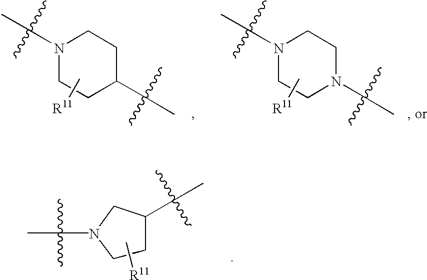

11. A compound according to claim 41 wherein:
B is

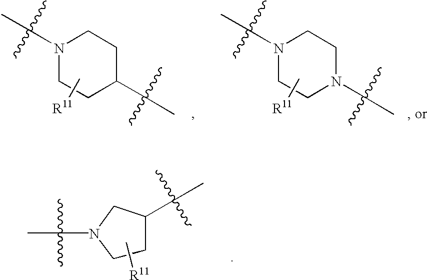

12. A compound according to claim 5, wherein:
B is

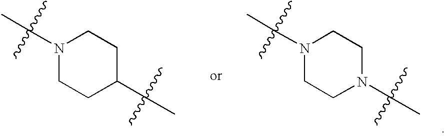

13. A compound according to claim 6, wherein:
B is

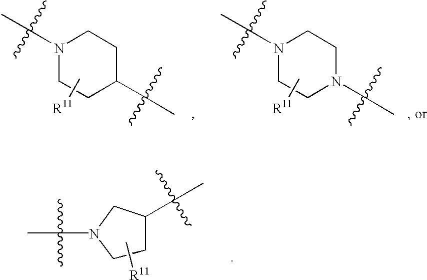

14. A compound according to claim 7, wherein:
B is

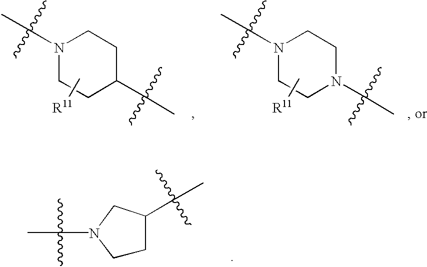

15. A compound selected from one of the Examples in Table 5a, Table 5b, Table 5c; Table 5d, Table 5e, Table 5f or Table 5g.

16. A pharmaceutical composition comprising a compound according to claim 1 and a pharmaceutically acceptable carrier.

17. A method for the treatment of neurological disorders associated with β-amyloid production comprising administering to a host in need of such treatment a therapeutically effective amount of a compound of claim 1.

18. A method for the treatment of Alzheimer's Disease associated with β-amyloid production comprising administering to a host in need of such treatment a therapeutically effective amount of a compound of claim 1.
